(19) United States
(12) Patent Application Publication
(10) Pub. No.: US 2023/0183344 A1
(43) Pub. Date: Jun. 15, 2023

(54) T-CELL BISPECIFIC BINDING PROTEINS

(71) Applicant: Magenta Therapeutics, Inc., Cambridge, MA (US)

(72) Inventors: Rahul Palchaudhuri, Somerville, MA (US); Bradley R. Pearse, Watertown, MA (US); Qing Li, Brookline, MA (US)

(21) Appl. No.: 17/906,471

(22) PCT Filed: Mar. 16, 2021
(86) PCT No.: PCT/US2021/022626

§ 371 Date: Sep. 15, 2022

Related U.S. Application Data
(60) Provisional application No. 62/990,281, filed on Mar. 16, 2020.

(51) Int. Cl. C07K 16/28

(52) U.S. Cl. CPC C07K 16/2809; C07K 2317/622; C07K 2317/526; C07K 2317/565; C07K 2317/31

(57) ABSTRACT

Provided herein are compositions and methods useful for the T-cell mediated depletion of hematopoietic stem cells (HSCs) expressing an HSC antigen, e.g., CD117+ cells, and for the treatment of various hematopoietic diseases, metabolic disorders, cancers, e.g., acute myeloid leukemia (AML) and autoimmune diseases, among others. Described herein are bispecific binding polypeptides, and bispecific antigen-binding portions thereof, comprising a first binding moiety to a T cell antigen, such as CD3, and a second binding moiety to an HSC antigen, such as CD117.

### RELATED APPLICATIONS

This application claims priority to U.S. Provisional Application No. 62/990,281, filed Mar. 16, 2020. The entire contents of the foregoing priority application are incorporated by reference herein.

### SEQUENCE LISTING

The instant application contains a Sequence Listing which has been submitted electronically in ASCII format and is hereby incorporated by reference in its entirety. Said ASCII copy, created on Mar. 16, 2021, is named M103034_2210WO_Sequence_Listing.txt and is 56,311 bytes in size.

### FIELD OF THE DISCLOSURE

The invention relates to the use of T-cell bispecific binding proteins, such as anti-CD3 bispecific antibodies, to mediate immune-driven depletion of target cells, including antigens expressed on HSC cells. The invention further relates to anti-CD117 bispecific binding proteins or fragments thereof compositions and uses thereof, comprising a first binding domain which binds to an antigen expressed on the surface of a hematopoietic cell, such as a hematopoietic stem cell, and a second binding domain which binds to a T cell.

### BACKGROUND OF THE DISCLOSURE

Selective cell depletion has potential for treatment of a number of therapies, including conditioning for stem cell transplantation, treatment of autoimmune diseases, and treatment of certain cancers. For example, B cell depletion therapy can be used to treat certain autoimmune conditions (Lee et al. (2020) Nature Reviews Drug Discovery, volume 20, pp. 179-199).

Conditioning is a process by which a patient is prepared (i.e., “conditioned”) to receive a transplant containing hematopoietic stem cells. Conditioning procedures thereby promote the engraftment of a hematopoietic stem cell transplant. Conditioning is performed prior to engraftment in order to create proper conditions (e.g., creation of stem cell niches) for the patient to receive the transplant. Further, in some situations, 20% engraftment of transplanted cells may alleviate or cure a particular disease state.

There are currently a number of non-specific (i.e., non-targeting) conditioning methods used in hematopoietic stem cell therapy (HSCT) indications and hemoglobinopathies, including, but not limited to, the use of irradiation (e.g., total body irradiation (TBI)) and DNA alkylating/modifying agents, both of which are highly toxic not only to the many of the patient's organ systems, but also affects hematopoietic and non-hematopoietic cells and the hematopoietic microenvironment. These harsh conditioning regimens typically result in the destruction of the recipient patient's immune system and niche cells, which can in many cases, lead to life-threatening complications.

Accordingly, the development of mild-conditioning regimens that selectively deplete an endogenous hematopoietic stem cell population in a target tissue while avoiding the undesirable toxicity of the aforementioned non-specific conditioning methods is needed. Depletion of stem cells, such as HSCs, can be facilitated by targeting certain molecules expressed on HSCs, including, for example, CD117.

CD117 (also referred to as c-kit or Stem Cell Factor Receptor (SCRF)) is a single transmembrane, receptor tyrosine kinase that binds the ligand Stem Cell Factor (SCF). SCF induces homodimerization of cKIT which activates its tyrosine kinase activity and signals through both the PI3-AKT and MAPK pathways (Kindblom et al., Am J. Path. 1998 152(5):1259). CD117 was initially discovered as an oncogene and has been studied in the field of oncology (see, for example, Stankov et al. (2014) Curr Pharm Des. 20(17):2849-80). CD117 is highly expressed on hematopoietic stem cells (HSCs). This expression pattern makes CD117 a potential target for conditioning across a broad range of diseases. There remains, however, a need for anti-CD117 based therapy that is effective for conditioning a patient for transplantation, such as a bone marrow transplantation.

There is currently a need for alternative methods and compositions that target stem cells, e.g., CD117+ stem cells that can be used as conditioning agents to promote the engraftment of exogenous stem cells. Such therapies and agents may also be useful for treating other diseases, where selective cell depletion would be therapeutic.

### SUMMARY OF THE DISCLOSURE

Described herein are methods and compositions relating to T cell bispecific binding proteins that mediate immune-driven depletion of target cells. In certain embodiments, the methods and compositions disclosed herein relate to a CD3 bispecific binding protein, such as a bispecific antibody, that also binds to a target antigen expressed on a stem cell, such as a hematopoietic stem cell (HSC). The advantage of the compositions and methods disclosed herein is that the bispecific depletes the target cell using T cells and reduces or eliminates the need for non-specific methods of cell depletion, such as chemotherapy or irradiation.

In one aspect, described herein are anti-CD117 bispecific binding proteins or fragments thereof comprising a first binding domain which binds to an antigen expressed on the surface of a hematopoietic cell, such as a hematopoietic stem cell (i.e., human CD117; also known as c-kit), and a second binding domain which binds to an antigen expressed on the surface of an immune cell, such as a T cell (e.g., CD3), as well as compositions and methods of using said bispecific binding proteins.

In one aspect, the present disclosure provides a bispecific binding polypeptide having a first antigen binding moiety that binds to CD117 expressed on a hematopoietic stem cell (HSC) or a hematopoietic progenitor cell; and a second antigen binding moiety that binds to an antigen expressed on a T cell. In on embodiment, the first antigen binding moiety is derived from an anti-CD117 antibody, or an antigen-binding fragment thereof. In another embodiment, the first antigen binding moiety comprises a single-chain variable fragment (scFv). In yet another embodiment, the first antigen binding moiety is selected from the group consisting of a Fab, a Fab′, a di-scFv, a tandem di-scFv, a tri-scFv, a tandem tri-scFv, a Fv, a disulfide linked Fv, a DART, a single domain antibody (sdAb), a diabody, a tandem diabody, a triabody and a tandem triabody. In another embodiment, the first antigen binding moiety comprises an anti-CD117 scFv.

In other embodiments, the anti-CD117 scFv comprises (i) a heavy chain variable region comprising a CDR1, a CDR2, and a CDR3 having an amino acid sequence as set forth in SEQ ID NOs: 7, 8, and 9, respectively, and comprises a light chain variable region comprising a CDR1, a CDR2, and a CDR3 having an amino acid sequence as set forth in SEQ ID NOs: 10, 11, and 12, respectively; or (ii) a heavy chain variable region comprising an amino acid sequence as set forth in SEQ ID NO: 13 and a light chain variable region comprising an amino acid sequence as set forth in SEQ ID NO: 14; or; (iii) a heavy chain variable region comprising a CDR1, a CDR2, and a CDR3 having an amino acid sequence as set forth in SEQ ID NOs: 21, 22, and 23, respectively, and comprises a light chain variable region comprising a CDR1, a CDR2, and a CDR3 having an amino acid sequence as set forth in SEQ ID NOs: 24, 25, and 26, respectively; or (iv) a heavy chain variable region comprising an amino acid sequence as set forth in SEQ ID NO: 27 and a light chain variable region comprising an amino acid sequence as set forth in SEQ ID NO: 28.

In certain embodiments, a bispecific anti-CD3/CD117 antibody comprises binding regions (e.g., VH and VL; or VH and VL CDRs) as described in the anti-CD117 antibody and the anti-CD3 antibody amino acid sequences described in Table 4.

In certain other embodiments, the second antigen binding moiety is derived from an antibody, or an antigen-binding fragment thereof. In one embodiment, the second antigen binding moiety comprises a single-chain variable fragment (scFv). In another embodiment, the second antigen binding moiety is selected from the group consisting of a Fab, a Fab′, a di-scFv, a tandem di-scFv, a tri-scFv, a tandem tri-scFv, a Fv, a DART, a disulfide linked Fv, a single domain antibody (sdAb), a diabody, a tandem diabody, a triabody and a tandem triabody. In yet another embodiment, the antigen expressed on the immune cell is CD3. In some embodiments, the CD3 is encoded by a gene selected form the group consisting of CD3D (CD3 δ), CD3E (CD3 ε), CD3G (CD3 γ), and CD3Z (CD3 ζ). In other embodiments, the second antigen binding moiety comprises an anti-CD3 scFv.

In other embodiments, the anti-CD3 scFv comprises an anti-CD117 VH amino acid sequence as set forth in SEQ ID NO: 37 and an anti-CD117 VL amino acid sequence as set forth in SEQ ID NO: 38.

In another embodiment, the first antigen binding moiety comprises a first single-chain variable fragment (scFv) and wherein the second antigen binding moiety comprises a second scFv. In yet another embodiment, the bispecific binding polypeptide is a tandem single-chain variable fragment (ta-scFv) comprising a first scFv and a second scFv. In certain other embodiments, the first scFv and the second scFv are connected by a linker. In some embodiments, the first scFv is an anti-CD117 scFv and wherein the second scFv is an anti-CD3 scFv.

In certain embodiments, the anti-CD117 scFv comprises (i) a heavy chain variable region comprising a CDR1, a CDR2, and a CDR3 having an amino acid sequence as set forth in SEQ ID NOs: 7, 8, and 9, respectively, and comprises a light chain variable region comprising a CDR1, a CDR2, and a CDR3 having an amino acid sequence as set forth in SEQ ID NOs: 10, 11, and 12, respectively; or (ii) a heavy chain variable region comprising an amino acid sequence as set forth in SEQ ID NO: 13 and a light chain variable region comprising an amino acid sequence as set forth in SEQ ID NO: 14; or; (iii) a heavy chain variable region comprising a CDR1, a CDR2, and a CDR3 having an amino acid sequence as set forth in SEQ ID NOs: 21, 22, and 23, respectively, and comprises a light chain variable region comprising a CDR1, a CDR2, and a CDR3 having an amino acid sequence as set forth in SEQ ID NOs: 24, 25, and 26, respectively; or (iv) a heavy chain variable region comprising an amino acid sequence as set forth in SEQ ID NO: 27 and a light chain variable region comprising an amino acid sequence as set forth in SEQ ID NO: 28. In another embodiments, the anti-CD3 scFv comprises an anti-CD117 VH amino acid sequence as set forth in SEQ ID NO: 37 and an anti-CD117 VL amino acid sequence as set forth in SEQ ID NO: 38.

In certain other embodiments, the bispecific binding polypeptide has an Fc region comprising a first Fc domain and a second Fc domain capable of stable association. In some embodiments, the Fc region is an isotype selected from the group consisting of IgG, IgA, IgM, IgD, and IgE. In other embodiments, the IgG is an IgG1 or an IgG4. In yet other embodiments, the Fc region comprises amino acid substitutions relative to a wild-type Fc region at positions L234, L235 (EU index), and D265 (EU index). In one embodiment, the amino acid substitution at position L234 is L234A. In another embodiment, the amino acid substitution at position L235 is L235A. In yet another embodiment, the bispecific binding polypeptide is a bispecific antibody, or a bispecific antigen-binding portion thereof.

In another aspect, the present disclosure provides a pharmaceutical composition having a therapeutically effective amount of a bispecific binding polypeptide, bispecific antibody, or a bispecific antigen-binding portion thereof as disclosed herein.

In another aspect, the present disclosure provides a method of treating a stem cell disorder in a human patient, by administering to the patient a therapeutically effective amount of a bispecific binding polypeptide, bispecific antibody, or a bispecific antigen-binding portion thereof as disclosed herein.

In another aspect, the present disclosure provides a method of treating an immunodeficiency disorder in a human patient by administering to the patient a therapeutically effective amount of a bispecific binding polypeptide, bispecific antibody, or a bispecific antigen-binding portion thereof as described herein. In some embodiments, the immunodeficiency disorder is a congenital immunodeficiency or an acquired immunodeficiency.

In another aspect, the present disclosure provides a method of treating a metabolic disorder in a human patient by administering to the patient a therapeutically effective amount of a bispecific binding polypeptide, bispecific antibody, or a bispecific antigen-binding portion thereof as disclosed herein. In one embodiment, the metabolic disorder is selected from the group consisting of glycogen storage diseases, mucopolysaccharidoses, Gaucher's Disease, Hurlers Disease, sphingolipidoses, and metachromatic leukodystrophy.

In another aspect, the present disclosure provides a method of treating an autoimmune disorder in a human patient by administering to the patient a therapeutically effective amount of a bispecific binding polypeptide, bispecific antibody, or a bispecific antigen-binding portion thereof as disclosed herein. In one embodiment, the autoimmune disorder is selected from the group consisting of multiple sclerosis, human systemic lupus, rheumatoid arthritis, inflammatory bowel disease, treating psoriasis, Type 1 diabetes mellitus, acute disseminated encephalomyelitis, Addison's disease, alopecia universalis, ankylosing spondylitis, antiphospholipid antibody syndrome, aplastic anemia, autoimmune hemolytic anemia, autoimmune hepatitis, autoimmune inner ear disease, autoimmune lymphoproliferative syndrome, autoimmune oophoritis, Balo disease, Behcet's disease, bullous pemphigoid, cardiomyopathy, Chagas' disease, chronic fatigue immune dysfunction syndrome, chronic inflammatory demyelinating polyneuropathy, Crohn's disease, cicatrical pemphigoid, coeliac sprue-dermatitis herpetiformis, cold agglutinin disease, CREST syndrome, Degos disease, discoid lupus, dysautonomia, endometriosis, essential mixed cryoglobulinemia, fibromyalgia-fibromyositis, Goodpasture's syndrome, Grave's disease, Guillain-Barre syndrome, Hashimoto's thyroiditis, Hidradenitis suppurativa, idiopathic and/or acute thrombocytopenic purpura, idiopathic pulmonary fibrosis, IgA neuropathy, interstitial cystitis, juvenile arthritis, Kawasaki's disease, lichen planus, Lyme disease, Meniere disease, mixed connective tissue disease, myasthenia gravis, neuromyotonia, opsoclonus myoclonus syndrome, optic neuritis, Ord's thyroiditis, pemphigus vulgaris, pernicious anemia, polychondritis, polymyositis and dermatomyositis, primary biliary cirrhosis, polyarteritis nodosa, polyglandular syndromes, polymyalgia rheumatica, primary agammaglobulinemia, Raynaud phenomenon, Reiter's syndrome, rheumatic fever, sarcoidosis, scleroderma, Sjögren's syndrome, stiff person syndrome, Takayasu's arteritis, temporal arteritis, ulcerative colitis, uveitis, vasculitis, vitiligo, vulvodynia, and Wegener's granulomatosis

In another aspect, the present disclosure provides a method of treating a cancer in a human patient, the method comprising administering to the patient a therapeutically effective amount of a bispecific binding polypeptide, bispecific antibody, or a bispecific antigen-binding portion thereof as disclosed herein. In one embodiment, the cancer is selected from the group consisting of leukemia, lymphoma, multiple myeloma, and neuroblastoma.

In another aspect, the present disclosure provides a method of depleting a population of stem cells in a human patient, the method comprising administering to the patient an effective amount of a bispecific binding polypeptide, bispecific antibody, or a bispecific antigen-binding portion thereof of as disclosed herein. In one embodiment, the method involves administering to the patient a transplant comprising hematopoietic stem cells.

Included herein is a bispecific binding polypeptide comprising a first antigen binding moiety that binds to CD117 expressed on a hematopoietic stem cell (HSC) or a hematopoietic progenitor cell; and a second antigen binding moiety that binds to an antigen expressed on a T cell.

In certain embodiments, the second antigen binding moiety binds to CD3.

In one embodiment, the first antigen binding moiety comprises an anti-CD117 single-chain variable fragment (scFv) and the second antigen binding moiety comprises an anti-CD3 scFv.

In one embodiment, the bispecific binding polypeptide is a bispecific antibody, or a bispecific antigen-binding fragment thereof.

In one embodiment, the anti-CD117 binding moiety comprises a heavy chain variable region comprising a CDR1, a CDR2, and a CDR3 having an amino acid sequence as set forth in SEQ ID NOs: 7, 8, and 9, respectively, and a light chain variable region comprising a CDR1, a CDR2, and a CDR3 having an amino acid sequence as set forth in SEQ ID NOs: 10, 11, and 12, respectively.

In one embodiment, the anti-CD117 binding moiety comprises a heavy chain variable region comprising an amino acid sequence as set forth in SEQ ID NO: 13 and a light chain variable region comprising an amino acid sequence as set forth in SEQ ID NO: 14.

In one embodiment, the anti-CD117 binding moiety comprises a heavy chain variable region comprising a CDR1, a CDR2, and a CDR3 having an amino acid sequence as set forth in SEQ ID NOs: 21, 22, and 23, respectively, and a light chain variable region comprising a CDR1, a CDR2, and a CDR3 having an amino acid sequence as set forth in SEQ ID NOs: 24, 25, and 26, respectively.

In one embodiment, the anti-CD117 binding moiety comprises a heavy chain variable region comprising an amino acid sequence as set forth in SEQ ID NO: 27 and a light chain variable region comprising an amino acid sequence as set forth in SEQ ID NO: 28.

In one embodiment, the anti-CD3 binding moiety comprises a heavy chain variable region comprising an amino acid sequence as set forth in SEQ ID NO: 37 and a light chain variable region comprising an amino acid sequence as set forth in SEQ ID NO: 38.

In one embodiment, the anti-CD3 binding moiety comprises a heavy chain variable region comprising an amino acid sequence as set forth in SEQ ID NO: 41 and a light chain variable region comprising an amino acid sequence as set forth in SEQ ID NO: 45.

Also disclosed herein is a bispecific antibody, or a bispecific antigen-binding portion thereof, comprising a CD117 binding region and a CD3 binding region, wherein the CD117 binding region comprises a heavy chain variable region comprising a CDR1, a CDR2, and a CDR3 having an amino acid sequence as set forth in SEQ ID NOs: 7, 8, and 9, respectively, and a light chain variable region comprising a CDR1, a CDR2, and a CDR3 having an amino acid sequence as set forth in SEQ ID NOs: 10, 11, and 12, respectively; a heavy chain variable region comprising an amino acid sequence as set forth in SEQ ID NO: 13 and a light chain variable region comprising an amino acid sequence as set forth in SEQ ID NO: 14; a heavy chain variable region comprising a CDR1, a CDR2, and a CDR3 having an amino acid sequence as set forth in SEQ ID NOs: 21, 22, and 23, respectively, and a light chain variable region comprising a CDR1, a CDR2, and a CDR3 having an amino acid sequence as set forth in SEQ ID NOs: 24, 25, and 26, respectively; or a heavy chain variable region comprising an amino acid sequence as set forth in SEQ ID NO: 27 and a light chain variable region comprising an amino acid sequence as set forth in SEQ ID NO: 28.

In one embodiment, the CD3 binding region of the bispecific antibody, or a bispecific antigen-binding portion thereof, comprises (i) an anti-CD117 VH amino acid sequence as set forth in SEQ ID NO: 37 and an anti-CD117 VL amino acid sequence as set forth in SEQ ID NO: 38; or (ii) a heavy chain variable region comprising an amino acid sequence as set forth in SEQ ID NO: 41 and a light chain variable region comprising an amino acid sequence as set forth in SEQ ID NO: 45.

In one embodiment, the bispecific antibody, or a bispecific antigen-binding portion thereof, comprises an Fc region comprising a first CH3 region of a first heavy chain and a second CH3 region of a second heavy chain, wherein the first and the second CH3 regions are capable of stable association via a knob-in-hole interaction.

In one embodiment, the bispecific antibody, or a bispecific antigen-binding portion thereof, is an isotype selected from the group consisting of IgG (e.g., IgG1 or an IgG4), IgA, IgM, IgD, and IgE.

In one embodiment, the Fc region of the bispecific antibody, or a bispecific antigen-binding portion thereof, comprises an amino acid substitution(s) relative to a wild-type Fc region at position L234, L235, H435, or combinations thereof (EU index). In one embodiment, the amino acid substitution at position L234 is L234A. In one embodiment, the amino acid substitution at position L235 is L235A. In one embodiment, the amino acid substitution at position H435 is H435A.

In certain embodiments, the bispecific antibody, or a bispecific antigen-binding portion thereof, comprises a first CH3 region comprising amino acid substitutions at positions T366, L368, and Y407 (EU index), and a second CH3 region comprising amino acid substitutions at position T366 (EU index). In one embodiment, the amino acid substitution at position T366 is T366S. In one embodiment, the amino acid substitution at position L368 is L368A. In one embodiment, the amino acid substitution at position Y407 is Y407V or Y407T. In one embodiment, the amino acid substitution at position T366 is T366W or T366Y.

Also disclosed is a pharmaceutical composition comprising a therapeutically effective amount of a bispecific binding polypeptide, bispecific antibody, or a bispecific antigen-binding portion thereof disclosed herein.

Further embodiments include a method of treating a stem cell disorder in a human patient, the method comprising administering to the patient a therapeutically effective amount of a bispecific binding polypeptide, bispecific antibody, or a bispecific antigen-binding portion thereof, disclosed herein.

Yet further embodiments include a method treating an immunodeficiency disorder in a human patient, the method comprising administering to the patient a therapeutically effective amount of a bispecific binding polypeptide, bispecific antibody, or a bispecific antigen-binding portion thereof, disclosed herein. In one embodiment, the immunodeficiency disorder is a congenital immunodeficiency or an acquired immunodeficiency.

Another embodiment includes a method of treating a metabolic disorder in a human patient, the method comprising administering to the patient a therapeutically effective amount of a bispecific binding polypeptide, bispecific antibody, or a bispecific antigen-binding portion thereof, as disclosed herein. In one embodiment, a metabolic disorder is selected from the group consisting of glycogen storage diseases, mucopolysaccharidoses, Gaucher's Disease, Hurlers Disease, sphingolipidoses, and metachromatic leukodystrophy

Yet another embodiment includes a method of treating an autoimmune disorder in a human patient, the method comprising administering to the patient a therapeutically effective amount of a bispecific binding polypeptide, bispecific antibody, or a bispecific antigen-binding portion thereof, as disclosed herein. Examples of an autoimmune disorder includes multiple sclerosis, human systemic lupus, rheumatoid arthritis, inflammatory bowel disease, treating psoriasis, Type 1 diabetes mellitus, acute disseminated encephalomyelitis, Addison's disease, alopecia universalis, ankylosing spondylitis, antiphospholipid antibody syndrome, aplastic anemia, autoimmune hemolytic anemia, autoimmune hepatitis, autoimmune inner ear disease, autoimmune lymphoproliferative syndrome, autoimmune oophoritis, Balo disease, Behcet's disease, bullous pemphigoid, cardiomyopathy, Chagas' disease, chronic fatigue immune dysfunction syndrome, chronic inflammatory demyelinating polyneuropathy, Crohn's disease, cicatrical pemphigoid, coeliac sprue-dermatitis herpetiformis, cold agglutinin disease, CREST syndrome, Degos disease, discoid lupus, dysautonomia, endometriosis, essential mixed cryoglobulinemia, fibromyalgia-fibromyositis, Goodpasture's syndrome, Grave's disease, Guillain-Barre syndrome, Hashimoto's thyroiditis, Hidradenitis suppurativa, idiopathic and/or acute thrombocytopenic purpura, idiopathic pulmonary fibrosis, IgA neuropathy, interstitial cystitis, juvenile arthritis, Kawasaki's disease, lichen planus, Lyme disease, Meniere disease, mixed connective tissue disease, myasthenia gravis, neuromyotonia, opsoclonus myoclonus syndrome, optic neuritis, Ord's thyroiditis, pemphigus vulgaris, pernicious anemia, polychondritis, polymyositis and dermatomyositis, primary biliary cirrhosis, polyarteritis nodosa, polyglandular syndromes, polymyalgia rheumatica, primary agammaglobulinemia, Raynaud phenomenon, Reiter's syndrome, rheumatic fever, sarcoidosis, scleroderma, Sjögren's syndrome, stiff person syndrome, Takayasu's arteritis, temporal arteritis, ulcerative colitis, uveitis, vasculitis, vitiligo, vulvodynia, or Wegener's granulomatosis

Yet another embodiment includes a method of treating a cancer in a human patient, the method comprising administering to the patient a therapeutically effective amount of a bispecific binding polypeptide, bispecific antibody, or a bispecific antigen-binding portion thereof, as disclosed herein. In one embodiment, the cancer is selected from the group consisting of leukemia, lymphoma, multiple myeloma, and neuroblastoma.

Still a further embodiment includes a method of depleting a population of stem cells in a human patient, the method comprising administering to the patient an effective amount of a bispecific binding polypeptide, bispecific antibody, or a bispecific antigen-binding portion thereof, as disclosed herein. In certain embodiments, the method further comprises administering to the patient a transplant comprising hematopoietic stem cells.

Also included herein is a method of selectively depleting hematopoietic stem cells (HSCs) in a human patient in need thereof, said method comprising administering a bispecific antibody, or a bispecific antigen-binding portion thereof, to the human subject in need thereof, such that HSCs are depleted, wherein the bispecific antibody, or a bispecific antigen-binding portion thereof, comprises a first binding moiety that specifically binds to a human HSC cell surface antigen and comprise a second binding moiety that specifically binds to a human T cell surface antigen. In one embodiment, the first antigen binding moiety binds to a human HSC cell surface antigen selected from the group consisting of CD7, CDwl2, CD13, CD15, CD19, CD21, CD22, CD29, CD30, CD33, CD34, CD36, CD38, CD40, CD41, CD42a, CD42b, CD42c, CD42d, CD43, CD48, CD49b, CD49d, CD49e, CD49f, CD50, CD53, CD55, CD64a, CD68, CD71, CD72, CD73, CD81, CD82, CD85A, CD85K, CD90, CD99, CD104, CD105, CD109, CD110, CD111, CD112, CD114, CD115, CD117, CD123, CD124, CD126, CD127, CD130, CD131, CD133, CD135, CD138, CD151, CD157, CD162, CD164, CD168, CD172a, CD173, CD174, CD175, CD175s, CD176, CD183, CD191, CD200, CD201, CD205, CD217, CD220, CD221, CD222, CD223, CD224, CD225, CD226, CD227, CD228, CD229, CD230, CD235a, CD235b, CD236, CD236R, CD238, CD240, CD242, CD243, CD277, CD292, CDw293, CD295, CD298, CD309, CD318, CD324, CD325, CD338, CD344, CD349 and CD350. In certain embodiments, the first antigen binding moiety binds to CD117. In certain embodiments, the second antigen binding moiety binds to human CD3.

In one embodiment, the first antigen binding moiety binds to CD117, and wherein the first antigen binding moiety comprises (i) a heavy chain variable region comprising a CDR1, a CDR2, and a CDR3 having an amino acid sequence as set forth in SEQ ID NOs: 7, 8, and 9, respectively, and comprises a light chain variable region comprising a CDR1, a CDR2, and a CDR3 having an amino acid sequence as set forth in SEQ ID NOs: 10, 11, and 12, respectively; or (ii) a heavy chain variable region comprising an amino acid sequence as set forth in SEQ ID NO: 13 and a light chain variable region comprising an amino acid sequence as set forth in SEQ ID NO: 14; or; (iii) a heavy chain variable region comprising a CDR1, a CDR2, and a CDR3 having an amino acid sequence as set forth in SEQ ID NOs: 21, 22, and 23, respectively, and comprises a light chain variable region comprising a CDR1, a CDR2, and a CDR3 having an amino acid sequence as set forth in SEQ ID NOs: 24, 25, and 26, respectively; or (iv) a heavy chain variable region comprising an amino acid sequence as set forth in SEQ ID NO: 27 and a light chain variable region comprising an amino acid sequence as set forth in SEQ ID NO: 28.

In one embodiment, the second antigen binding moiety binds to CD3, and wherein the second antigen binding moiety comprises (i) a heavy chain variable region comprising an amino acid sequence as set forth in SEQ ID NO: 37 and a light chain variable region comprising an amino acid sequence as set forth in SEQ ID NO: 38; or (ii) a heavy chain variable region comprising an amino acid sequence as set forth in SEQ ID NO: 41 and a light chain variable region comprising an amino acid sequence as set forth in SEQ ID NO: 45.

In one embodiment, the bispecific antibody, or the bispecific antigen-binding fragment thereof, is an IgG, e.g., an IgG1 or an IgG4.

In certain embodiments, the bispecific antibody, or the bispecific antigen binding fragment thereof, comprises an Fc region comprising a first CH3 region of a first heavy chain, and comprises a second CH3 region of a second heavy chain, wherein the first and the second CH3 regions are capable of stable association via a knob-in-hole interaction. In one embodiment, the first CH3 region comprises amino acid substitutions at positions T366, L368, and Y407 (EU index), and the second CH3 region comprises amino acid substitutions at position T366 (EU index). In one embodiment, the amino acid substitution at position T366 is T366S. In one embodiment, the amino acid substitution at position L368 is L368A. In one embodiment, the amino acid substitution at position Y407 is Y407V or Y407T. In one embodiment, the amino acid substitution at position T366 is T366W or T366Y.

In one embodiment, the patient has a stem cell disorder and is in need of a transplant. In one embodiment, the method further comprises administering an HSC transplant to the patient following depletion.

In one embodiment, the patient has an immunodeficiency disorder, a metabolic disorder, an autoimmune disorder, or cancer.

### BRIEF DESCRIPTION OF THE FIGURES

FIG. 1 graphically depicts the results of an in vitro cell killing assay using CD117 expressing target cells (Kasumi-1 cells) for an anti-CD117/CD3 bispecific antibody (i.e., “bs-Ab-1”) in comparison to antibody controls having one non-targeting arm and one targeting arm (i.e., either a CD3 or a CD117 targeting arm). The control antibodies are referred to as “Anti-CD17-Isotype antibody” and “Anti-CD3-Isotype antibody” in FIG. 1.

FIG. 2 graphically depict the results of in vitro cell killing assays using primary human hematopoietic stem cells for the bs-Ab-1 bispecific antibody in comparison to antibody controls having one non-targeting arm and one targeting arm (i.e., either a CD3 or CD117 targeting arm). The controls are referred to as “Anti-CD117-Isotype antibody” and “Anti-CD3-Isotype antibody” in FIG. 2.

FIGS. 3A to 3D graphically depict the results of an in vivo cell depletion assay that shows the bs-Ab-1 bispecific antibody selectively depletes human HSCs in humanized NSG mice. FIG. 3A shows the frequency (%) of CD34+ cells maintained after 21 days in mice treated with various concentrations of a single dose of (i) the bs-Ab-1 bispecific antibody, (ii) an anti-CD117-Isotype antibody, (iii) an anti-CD3-Isotype antibody, (iv) a combination of an anti-CD117-Isotype antibody and an anti-CD3-Isotype antibody, and (v) a control (i.e., “PBS”). FIG. 3B shows the absolute number of CD34+ cells maintained after 21 days in mice treated with various concentrations of a single dose of (i) the bs-Ab-1 bispecific antibody, (ii) an anti-CD117-Isotype antibody, (iii) an anti-CD3-Isotype antibody, (iv) a combination of an anti-CD117-Isotype antibody and an anti-CD3-Isotype antibody, and (v) a control (i.e., “PBS”). FIG. 3C shows the frequency (%) of CD34+CD117+ cells maintained after 21 days in mice treated with various concentrations of a single dose of (i) the bs-Ab-1 bispecific antibody, (ii) an anti-CD117-Isotype antibody, (iii) an anti-CD3-Isotype antibody, (iv) a combination of an anti-CD117-Isotype antibody and an anti-CD3-Isotype antibody, and (v) a control (i.e., “PBS”). FIG. 3D shows the absolute number of CD34+CD117+ cells maintained after 21 days in mice treated with various concentrations of a single dose of (i) a bs-Ab-1 bispecific antibody, (ii) an anti-CD117-Isotype antibody, (iii) an anti-CD3-Isotype antibody, (iv) a combination of an anti-CD117-Isotype antibody and an anti-CD3-Isotype antibody, and (v) a control (i.e., “PBS”). As described above, anti-CD3-Isotype antibody and anti-CD117-Isotype antibody refer to control antibodies having either a CD3 or a CD117 targeting arm and a non-targeting arm.

FIG. 4 graphically depicts the results of an in vitro cell killing assay using primary human stem cells for the bs-Ab-2 bispecific antibody and bs-Ab-3 bispecific antibody in comparison to (i) a combination of an Ab85-Isotype antibody (i.e., an antibody with a CD117 targeting arm of Ab85 (with the T366Y and H435A amino acid substitutions) and a non-targeting arm (with the Y407T and H435A amino acid substitutions); referred to as “Ab85-Iso” in FIG. 4) and an anti-CD3-Isotype antibody (i.e., an antibody with a CD3 targeting arm of Ab2 (with the Y407T and H435A amino acid substitutions) and a non-targeting arm (with the T366Y and H435A amino acid substitutions); referred to as “anti-CD3-Iso” in FIG. 4) and (ii) a combination of an Ab67-Isotype (i.e., an antibody with a CD117 targeting arm of Ab67 (with the T366Y and H435A amino acid substitutions) and a non-targeting arm (with the Y407T and H435A amino acid substitutions); referred to as “Ab65-Iso” in FIG. 4) and an anti-CD3-Isotype antibody (i.e., an antibody with a CD3 targeting arm of Ab2 (with the Y407T and H435A amino acid substitutions) and a non-targeting arm (with the T366Y and H435A amino acid substitutions); referred to as “anti-CD3-Iso” in FIG. 4).

FIG. 5 provides a schematic of a T-cell bispecific antibody which can be used to mediate immune-driven depletion of target cells. The bispecific antibody in FIG. 5 has a T cell binding arm (heavy and light chain) that binds to CD3 and a target cell (e.g., HSC) binding arm (heavy and light chain) that binds to CD117.

### DETAILED DESCRIPTION

Described herein are bispecifics that can be used to mediate cell depletion via T cells. The methods and compositions disclosed herein use T-cells bound by a T-cell specific bispecific protein to deplete target cells, where the target is defined by the second arm of the bispecific protein. For example, a bispecific may target CD3 (T cell antigen) and CD117 (an HSC target antigen). Thus, included herein are methods and compositions relating to anti-CD117 bispecific binding proteins or fragments thereof that bind to human CD117 and human CD3.

Generally, HSC and CD3 targeting bispecific proteins disclosed herein, e.g., anti-CD117 bispecific binding proteins or fragments thereof provided herein have many characteristics making them advantageous for therapy, including methods of conditioning human patients for stem cell transplantation. These features also make the anti-CD117 bispecific binding proteins or fragments thereof disclosed herein advantageous for use in methods of treating patients suffering from various pathologies, such as blood diseases, metabolic disorders, cancers, and autoimmune diseases, among others.

The disclosure provides anti-CD117 bispecific binding proteins or fragments thereof that bind to the ectodomain of human CD117 and bind to human CD3 on the surface of a T cell. The binding regions of certain embodiments of the isolated anti-CD117 bispecific binding proteins or fragments thereof identified herein are described below.

HSC and CD3 targeting bispecific proteins disclosed herein (e.g., anti-CD117 bispecific binding proteins or fragments thereof described herein), can be used in methods of treating a variety of disorders, such as diseases of a cell type in the hematopoietic lineage, cancers, autoimmune diseases, metabolic disorders, and stem cell disorders, among others. The compositions and methods described herein may (i) directly deplete a population of cells that give rise to a pathology, such as a population of cancer cells (e.g., leukemia cells) and autoimmune cells (e.g., autoreactive T-cells), and/or (ii) deplete a population of endogenous hematopoietic stem cells so as to promote the engraftment of transplanted hematopoietic stem cells by providing a niche to which the transplanted cells may home. The foregoing activities can be achieved by administration of, for example, anti-CD117 bispecific binding proteins or fragments thereof that are capable of binding an antigen expressed by a hematopoietic stem cell (or an endogenous disease-causing cell), i.e., CD117, and an antigen expressed by an immune cell, such as a T cell, e.g., CD3. In the case of direct treatment of a disease, this administration can cause a reduction in the quantity of the cells that give rise to the pathology of interest. In the case of preparing a patient for hematopoietic stem cell transplant therapy, this administration can cause the selective depletion of a population of endogenous hematopoietic stem cells, thereby creating a vacancy in the hematopoietic tissue, such as the bone marrow, that can subsequently be filled by transplanted, exogenous hematopoietic stem cells. The invention is based in part on the discovery that anti-CD117 bispecific binding proteins or fragments thereof capable of binding CD117 (such as GNNK+ CD117) and CD3 can be administered to a patient to affect both of the above activities. Anti-CD117 bispecific binding proteins or fragments thereof, that bind CD117 and CD3 can be administered to a patient suffering from a cancer or autoimmune disease to directly deplete a population of cancerous cells or autoimmune cells, and can also be administered to a patient in need of hematopoietic stem cell transplant therapy in order to promote the survival and engraftment potential of transplanted hematopoietic stem cells.

Engraftment of hematopoietic stem cell transplants due to the administration of a bispecific binding protein that binds to CD3 and an anti-HSC antigen, e.g., anti-CD117 bispecific binding proteins or fragments thereof, can manifest in a variety of empirical measurements. For instance, engraftment of transplanted hematopoietic stem cells can be evaluated by assessing the quantity of competitive repopulating units (CRU) present within the bone marrow of a patient following administration of, e.g., an anti-CD117 bispecific binding proteins or fragments thereof capable of binding CD117 and CD3, and subsequent administration of a hematopoietic stem cell transplant. Additionally, one can observe engraftment of a hematopoietic stem cell transplant by incorporating a reporter gene, such as an enzyme that catalyzes a chemical reaction yielding a fluorescent, chromophoric, or luminescent product, into a vector with which the donor hematopoietic stem cells have been transfected and subsequently monitoring the corresponding signal in a tissue into which the hematopoietic stem cells have homed, such as the bone marrow. One can also observe hematopoietic stem cell engraftment by evaluation of the quantity and survival of hematopoietic stem and progenitor cells, for instance, as determined by fluorescence activated cell sorting (FACS) analysis methods known in the art. Engraftment can also be determined by measuring white blood cell counts in peripheral blood during a post-transplant period, and/or by measuring recovery of marrow cells by donor cells in a bone marrow aspirate sample.

The sections that follow provide a description of bispecific binding proteins that bind to a T cell antigen, e.g., CD3, and a stem cell target, such as CD117. Examples include anti-CD117 bispecific binding proteins or fragments thereof. Compositions and methods disclosed herein can be used to treat a patient, such as a patient suffering from a cancer (such as acute myelogenous leukemia or myelodysplastic syndrome) or autoimmune disease, or a patient in need of hematopoietic stem cell transplant therapy in order to promote engraftment of hematopoietic stem cell grafts, as well as methods of administering such therapeutics to a patient (e.g., prior to hematopoietic stem cell transplantation).

### Definitions

As used herein, the term “about” refers to a value that is within 5% above or below the value being described.

As used herein, the term “antibody” refers to an immunoglobulin molecule that specifically binds to, or is immunologically reactive with, a particular antigen. An antibody includes, but is not limited to, monoclonal antibodies, polyclonal antibodies, multispecific antibodies (e.g., bispecific antibodies), genetically engineered antibodies, and otherwise modified forms of antibodies, including but not limited to de-immunized antibodies, chimeric antibodies, humanized antibodies, heteroconjugate antibodies (e.g., bi- tri- and quad-specific antibodies, diabodies, triabodies, and tetrabodies), and antibody fragments (i.e., antigen binding fragments of antibodies), including, for example, Fab′, F(ab′)2, Fab, Fv, rIgG, and scFv fragments, so long as they exhibit the desired antigen-binding activity.

Generally, antibodies comprise heavy and light chains containing antigen binding regions (also referred to herein as antigen binding moieties). Each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH, and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxyl-terminus in the following order: FR1, CDR1, FR2, CDR2, FR3, CDR3, FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (Clq) of the classical complement system.

The term “antigen-binding fragment,” as used herein, refers to one or more portions of an antibody that retain the ability to specifically bind to a target antigen. The antigen-binding function of an antibody can be performed by fragments of a full-length antibody. The antibody fragments can be, for example, a Fab, F(ab′)2, scFv, diabody, a triabody, an affibody, a nanobody, an aptamer, or a domain antibody. Examples of binding fragments encompassed of the term “antigen-binding fragment” of an antibody include, but are not limited to: (i) a Fab fragment, a monovalent fragment consisting of the VL, VH, CL, and CH1 domains; (ii) a F(ab′)2 fragment, a bivalent fragment containing two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb including VH and VL domains; (vi) a dAb fragment that consists of a VH domain (see, e.g., Ward et al., Nature 341:544-546, 1989); (vii) a dAb which consists of a VH or a VL domain; (viii) an isolated complementarity determining region (CDR); and (ix) a combination of two or more (e.g., two, three, four, five, or six) isolated CDRs which may optionally be joined by a synthetic linker. Furthermore, although the two domains of the Fv fragment, VL and VH, are coded for by separate genes, they can be joined, using recombinant methods, by a linker that enables them to be made as a single protein chain in which the VL and VH regions pair to form monovalent molecules (known as single chain Fv (scFv); see, for example, Bird et al., Science 242:423-426, 1988 and Huston et al., Proc. Natl. Acad. Sci. USA 85:5879-5883, 1988). These antibody fragments can be obtained using conventional techniques known to those of skill in the art, and the fragments can be screened for utility in the same manner as intact antibodies. Antigen-binding fragments can be produced by recombinant DNA techniques, enzymatic or chemical cleavage of intact immunoglobulins, or, in certain cases, by chemical peptide synthesis procedures known in the art. In the context of a bispecific antibody, an antigen-binding fragment would be a bispecific fragment, i.e., a bispecific antigen binding fragment (or portion).

An “intact” or “full length” antibody, as used herein, refers to an antibody having two heavy (H) chain polypeptides and two light (L) chain polypeptides interconnected by disulfide bonds. A bispecific antibody can be an intact antibody, where the first arm of the bispecific antibody comprises a light chain and a heavy chain that bind to a first antigen (or epitope), and the second arm of the bispecific antibody comprises a heavy chain and a light chain that bind to a second antigen (or epitope).

The term “specifically binds”, as used herein, refers to the ability of an antibody or bispecific binding protein to recognize and bind to a specific protein structure (epitope) rather than to proteins generally. If an antibody or bispecific binding protein is specific for epitope “A”, the presence of a molecule containing epitope A (or free, unlabeled A), in a reaction containing labeled “A” and the antibody, will reduce the amount of labeled A bound to the antibody. By way of example, an antibody “binds specifically” to a target if the antibody, when labeled, can be competed away from its target by the corresponding non-labeled antibody. In one embodiment, an antibody or bispecific binding protein specifically binds to a target, e.g., an antigen expressed by hematopoietic stem cells, such as CD117; if the antibody has a K_{D} for the target of at least about 10⁻⁴ M, about 10⁻⁵ M, about 10⁻⁶ M, about 10⁻⁷ M, about 10⁻⁸ M, about 10⁻⁹ M, about 10⁻¹⁰ M, about 10⁻¹¹ M, about 10⁻¹² M, or less (less meaning a number that is less than about 10⁻¹², e.g. 10⁻¹³). In one embodiment, K_{D} is determined according to standard bio-layer interferometery (BLI). It shall be understood, however, that the antibody may be capable of specifically binding to two or more antigens which are related in sequence. For example, in one embodiment, an antibody can specifically bind to both human and a non-human (e.g., mouse or non-human primate) orthologs of an antigen, e.g., CD117.

The term “monoclonal antibody” as used herein refers to an antibody that is derived from a single clone, including any eukaryotic, prokaryotic, or phage clone, by any means available or known in the art, and is not limited to antibodies produced through hybridoma technology. Monoclonal antibodies useful with the present disclosure can be prepared using a wide variety of techniques known in the art including the use of hybridoma, recombinant, and phage display technologies, or a combination thereof.

As used herein, the term “immune cell” is intended to include, but is not limited to, a cell that is of hematopoietic origin and that plays a role in the immune response. Immune cells include, but are not limited to, T cells and natural killer (NK) cells. Natural killer cells are well known in the art. In one embodiment, natural killer cells include cell lines, such as NK-92 cells. Further examples of NK cell lines include NKG, YT, NK-YS, HANK-1, YTS cells, and NKL cells. An immune cell can be allogeneic or autologous.

As used herein, the term “anti-CD117 antibody” or “an antibody that binds to CD117” refers to an antibody that is capable of binding CD117 with sufficient affinity such that the antibody is useful as a diagnostic and/or therapeutic agent in targeting CD117. Likewise, the term “anti-CD117 bispecific binding protein” or “a bispecific binding protein that binds to CD117” refers to a bispecific binding protein that is capable of binding CD117 with sufficient affinity such that the bispecific binding protein is useful as a diagnostic and/or therapeutic agent in targeting CD117.

As used herein, the term “bispecific binding proteins” or “bispecific binding polypeptide” refers to a protein comprising antigen binding regions that bind to two antigens (or two epitopes on the same antigen). An example of a bispecific binding protein is a bispecific antibody or a BiTE (see, e.g., Einsele et al. (2020) Cancer vol. 126(14): 3192-3201).

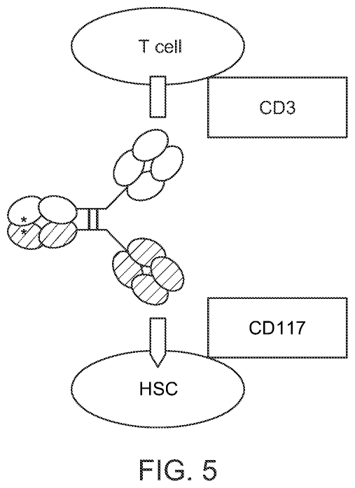

The term “bispecific antibody” or “bispecific antibody construct” refers to to an antibody that displays dual binding specificity for two different antigens or two different epitopes, where each binding site differs and recognizes a different antigen or epitope. For instance, one of the binding specificities can be directed towards an epitope on a hematopoietic stem cell (HSC) surface antigen, e.g., CD117 (e.g., GNNK+ CD117), and the other can be directed towards an epitope on a different cell, such as an immune cell (e.g., a T cell) or an epitope on a different hematopoietic stem cell surface antigen or another cell surface protein, such as a receptor or receptor subunit involved in a signal transduction pathway that potentiates cell growth, among others. In one embodiment, a bispecific antibody is represented by the intact antibody described in FIG. 5, where the bispecific antibody contains a T cell specific binding arm comprising an antibody heavy and a light chain, and contains a second target specific binding arm (e.g., binds to an antigen expressed on an HSC) comprising an antibody heavy and a light chain.

In a particular embodiment, the “bispecific binding protein” or “bispecific antibody” or “bispecific antibody construct” has a first antigen binding domain (or binding moiety) that binds to CD117 and has a second antigen binding domain (or binding moiety) that binds to CD3.

Given that the bispecific binding proteins, e.g., anti-CD117 bispecific binding proteins or fragments thereof disclosed herein are (at least) bispecific, they do not occur naturally and they are markedly different from naturally occurring products. A “bispecific” binding protein or immunoglobulin is hence an artificial hybrid antibody or immunoglobulin having at least two distinct binding sites with different specificities. Bispecific binding proteins can be produced by a variety of methods including fusion of hybridomas or linking of Fab′ fragments. See, e.g., Songsivilai & Lachmann, Clin. Exp. Imunol. 79:315-321 (1990).

The term “knob-in-hole” or “knobs in hole” refers to a certain type of bispecific antibody which contains a first arm containing a light and a heavy chain that binds to a first antigen (or epitope) and a second arm containing a second light and heavy chain that binds to a second antigen (or epitope). A knob-in-hole bispecific antibody involves engineering CH3 domains of each arm to create either a “knob” or a “hole” in each heavy chain to promote heterodimerization of the two heavy chains.

According to a particular embodiment, a T-cell/HSC specific bispecific (e.g., anti-CD3/CD117 bispecific antibody) is a “bispecific single chain binding protein”, more preferably a bispecific “single chain Fv” (scFv). Although the two domains of the Fv fragment, VL and VH, are coded for by separate genes, they can be joined, using recombinant methods, by a synthetic linker that enables them to be made as a single protein chain in which the VL and VH regions pair to form a monovalent molecule; see e.g., Huston et al. (1988) Proc. Natl. Acad. Sci USA 85:5879-5883). These antibody fragments are obtained using conventional techniques known to those with skill in the art, and the fragments are evaluated for function in the same manner as are whole or full-length antibodies. A single-chain variable fragment (scFv) is hence a fusion protein of the variable region of the heavy chain (VH) and of the light chain (VL) of immunoglobulins, usually connected with a short linker peptide of about ten to about 25 amino acids, preferably about 15 to 20 amino acids. The linker is usually rich in glycine for flexibility, as well as serine or threonine for solubility, and can either connect the N-terminus of the VH with the C-terminus of the VL, or vice versa. This protein retains the specificity of the original immunoglobulin, despite removal of the constant regions and introduction of the linker.

As used herein, the term “complementarity determining region” (CDR) refers to a hypervariable region found both in the light chain and the heavy chain variable domains of an antibody (or bispecific binding protein as described herein). The more highly conserved portions of variable domains are referred to as framework regions (FRs). The amino acid positions that delineate a hypervariable region of an antibody (or bispecific binding protein as described herein) can vary, depending on the context and the various definitions known in the art. Some positions within a variable domain may be viewed as hybrid hypervariable positions in that these positions can be deemed to be within a hypervariable region under one set of criteria while being deemed to be outside a hypervariable region under a different set of criteria. One or more of these positions can also be found in extended hypervariable regions. The antibodies (or bispecific binding protein as described herein) described herein may contain modifications in these hybrid hypervariable positions. The variable domains of native heavy and light chains each contain four framework regions that primarily adopt a β-sheet configuration, connected by three CDRs, which form loops that connect, and in some cases form part of, the β-sheet structure. The CDRs in each chain are held together in close proximity by the framework regions in the order FR1-CDR1-FR2-CDR2-FR3-CDR3-FR4 and, with the CDRs from the other antibody chains, contribute to the formation of the target binding site of antibodies (see Kabat et al., Sequences of Proteins of Immunological Interest, National Institute of Health, Bethesda, Md., 1987). In certain embodiments, numbering of immunoglobulin amino acid residues is performed according to the immunoglobulin amino acid residue numbering system of Kabat et al., unless otherwise indicated (although any antibody numbering scheme, including, but not limited to IMGT and Chothia, can be utilized).

The terms “Fc”, “Fc region,” “Fc domain,” and “IgG Fc domain” as used herein refer to the portion of an immunoglobulin, e.g., an IgG molecule, that correlates to a crystallizable fragment obtained by papain digestion of an IgG molecule. The Fc region comprises the C-terminal half of two heavy chains of an IgG molecule that are linked by disulfide bonds. It has no antigen binding activity but contains the carbohydrate moiety and binding sites for complement and Fc receptors, including the FcRn receptor (see below). For example, an Fc domain contains the second constant domain CH2 (e.g., residues at EU positions 231-340 of human IgG1) and the third constant domain CH3 (e.g., residues at EU positions 341-447 of human IgG1). As used herein, the Fc domain includes the “lower hinge region” (e.g., residues at EU positions 233-239 of human IgG1).

Fc can refer to this region in isolation, or this region in the context of a bispecific binding protein, an antibody, antibody fragment, or Fc fusion protein. Polymorphisms have been observed at a number of positions in Fc domains, including but not limited to EU positions 270, 272, 312, 315, 356, and 358, and thus slight differences between the sequences presented in the instant application and sequences known in the art can exist. Thus, a “wild type IgG Fc domain” or “WT IgG Fc domain” refers to any naturally occurring IgG Fc region (i.e., any allele). The sequences of the heavy chains of human IgG1, IgG2, IgG3 and IgG4 can be found in a number of sequence databases, for example, at the Uniprot database (www.uniprot.org) under accession numbers P01857 (IGHG1_HUMAN), P01859 (IGHG2_HUMAN), P01860 (IGHG3_HUMAN), and P01861 (IGHG1_HUMAN), respectively.

The terms “modified Fc region” or “variant Fc region” as used herein refers to an IgG Fc domain comprising one or more amino acid substitutions, deletions, insertions or modifications introduced at any position within the Fc domain. In certain aspects a variant IgG Fc domain comprises one or more amino acid substitutions resulting in decreased or ablated binding affinity for an Fc gamma R and/or Clq as compared to the wild type Fc domain not comprising the one or more amino acid substitutions. Further, Fc binding interactions are essential for a variety of effector functions and downstream signaling events including, but not limited to, antibody dependent cell-mediated cytotoxicity (ADCC) and complement dependent cytotoxicity (CDC). Accordingly, in certain aspects, an antibody comprising a variant Fc domain (e.g., an antibody, fusion protein or conjugate) can exhibit altered binding affinity for at least one or more Fc ligands (e.g., Fc gamma Rs) relative to a corresponding antibody otherwise having the same amino acid sequence but not comprising the one or more amino acid substitution, deletion, insertion or modifications such as, for example, an unmodified Fc region containing naturally occurring amino acid residues at the corresponding position in the Fc region.

Variant Fc domains are defined according to the amino acid modifications that compose them. For all amino acid substitutions discussed herein in regard to the Fc region, numbering is always according to the EU index as in Kabat. Thus, for example, D265C is an Fc variant with the aspartic acid (D) at EU position 265 substituted with cysteine (C) relative to the parent Fc domain. It is noted that the order in which substitutions are provided is arbitrary. Likewise, e.g., D265C/L234A/L235A defines a variant Fc variant with substitutions at EU positions 265 (D to C), 234 (L to A), and 235 (L to A) relative to the parent Fc domain. A variant can also be designated according to its final amino acid composition in the mutated EU amino acid positions. For example, the L234A/L235A mutant can be referred to as “LALA”. As a further example, the E233P.L234V.L235A.delG236 (deletion of 236) mutant can be referred to as “EPLVLAdeIG”. As yet another example, the 1253A.H310A.H435A mutant can be referred to as “IHH”. It is noted that the order in which substitutions are provided is arbitrary.

The terms “Fc gamma receptor” or “Fc gamma R” as used herein refer to any member of the family of proteins that bind the IgG antibody Fc region and are encoded by the Fc gamma R genes. In humans this family includes but is not limited to Fc gamma RI (CD64), including isoforms Fc gamma RIa, Fc gamma RIb, and Fc gamma RIc; Fc gamma RII (CD32), including isoforms Fc gamma RIIa (including allotypes H131 and R131), Fc gamma RIIb (including Fc gamma RIIb-1 and Fc gamma RIIb-2), and Fc gamma RIIc; and Fc gamma RIII (CD16), including isoforms Fc gamma RIIIa (including allotypes V158 and F158) and Fc gamma RIIIb (including allotypes Fc gamma RIIIb-NA1 and Fc gamma RIIIb-NA2), as well as any undiscovered human Fc gamma Rs or Fc gamma R isoforms or allotypes. An Fc gamma R can be from any organism, including but not limited to humans, mice, rats, rabbits, and monkeys. Mouse Fc gamma Rs include but are not limited to Fc gamma RI (CD64), Fc gamma RII (CD32), Fc gamma RIII (CD16), and Fc gamma RIII-2 (CD16-2), as well as any undiscovered mouse Fc gamma Rs or Fc gamma R isoforms or allotypes.

The term “effector function” as used herein refers to a biochemical event that results from the interaction of an Fc domain with an Fc receptor. Effector functions include but are not limited to ADCC, ADCP, and CDC. By “effector cell” as used herein is meant a cell of the immune system that expresses or one or more Fc receptors and mediates one or more effector functions. Effector cells include but are not limited to monocytes, macrophages, neutrophils, dendritic cells, eosinophils, mast cells, platelets, B cells, large granular lymphocytes, Langerhans' cells, natural killer (NK) cells, and gamma delta T cells, and can be from any organism included but not limited to humans, mice, rats, rabbits, and monkeys.

The term “silent”, “silenced”, or “silencing” as used herein refers to an antibody or bispecific binding protein having a modified Fc region described herein that has decreased binding to an Fc gamma receptor (FcγR) relative to binding of an identical antibody or bispecific binding protein comprising an unmodified Fc region to the FcγR (e.g., a decrease in binding to a FcγR by at least 70%, at least 80%, at least 90%, at least 95%, at least 98%, at least 99%, or 100% relative to binding of the identical antibody comprising an unmodified Fc region to the FcγR as measured by, e.g., BLI). In some embodiments, the Fc silenced antibody or bispecific binding protein has no detectable binding to an FcγR. Binding of an antibody having a modified Fc region to an FcγR can be determined using a variety of techniques known in the art, for example but not limited to, equilibrium methods (e.g., enzyme-linked immunoabsorbent assay (ELISA); KinExA, Rathanaswami et al. Analytical Biochemistry, Vol. 373:52-60, 2008; or radioimmunoassay (RIA)), or by a surface plasmon resonance assay or other mechanism of kinetics-based assay (e.g., BIACORE™ analysis or Octet™ analysis (forteBIO)), and other methods such as indirect binding assays, competitive binding assays fluorescence resonance energy transfer (FRET), gel electrophoresis and chromatography (e.g., gel filtration). These and other methods may utilize a label on one or more of the components being examined and/or employ a variety of detection methods including but not limited to chromogenic, fluorescent, luminescent, or isotopic labels. A detailed description of binding affinities and kinetics can be found in Paul, W. E., ed., Fundamental Immunology, 4th Ed., Lippincott-Raven, Philadelphia (1999), which focuses on antibody-immunogen interactions. One example of a competitive binding assay is a radioimmunoassay comprising the incubation of labeled antigen with the antibody of interest in the presence of increasing amounts of unlabeled antigen, and the detection of the antibody bound to the labeled antigen. The affinity of the antibody of interest for a particular antigen and the binding off-rates can be determined from the data by scatchard plot analysis. Competition with a second antibody can also be determined using radioimmunoassays. In this case, the antigen is incubated with antibody of interest conjugated to a labeled compound in the presence of increasing amounts of an unlabeled second antibody.

As used herein, the term “identical antibody comprising an unmodified Fc region” or “identical bispecific binding protein comprising an unmodified Fc region” refers to an antibody or bispecific binding protein that lacks the recited amino acid substitutions (e.g., D265C, H435A, L234A, and/or L235A), but otherwise has the same amino acid sequence as the Fc modified antibody or Fc modified bispecific binding protein to which it is being compared.

The terms “antibody-dependent cell-mediated cytotoxicity” or “ADCC” refer to a form of cytotoxicity in which a polypeptide comprising an Fc domain, e.g., an antibody or bispecific binding protein, bound onto Fc receptors (FcRs) present on certain cytotoxic cells (e.g., primarily NK cells, neutrophils, and macrophages) and enables these cytotoxic effector cells to bind specifically to an antigen-bearing “target cell” and subsequently kill the target cell with cytotoxins. (Hogarth et al., Nature review Drug Discovery 2012, 11:313) It is contemplated that, in addition to antibodies or bispecific binding proteins and fragments thereof, other polypeptides comprising Fc domains, e.g., Fc fusion proteins and Fc conjugate proteins, having the capacity to bind specifically to an antigen-bearing target cell will be able to effect cell-mediated cytotoxicity.

For simplicity, the cell-mediated cytotoxicity resulting from the activity of a polypeptide comprising an Fc domain is also referred to herein as ADCC activity. The ability of any particular polypeptide of the present disclosure to mediate lysis of the target cell by ADCC can be assayed. To assess ADCC activity, a polypeptide of interest (e.g., an antibody) is added to target cells in combination with immune effector cells, resulting in cytolysis of the target cell. Cytolysis is generally detected by the release of label (e.g., radioactive substrates, fluorescent dyes or natural intracellular proteins) from the lysed cells. Useful effector cells for such assays include peripheral blood mononuclear cells (PBMC) and Natural Killer (NK) cells. Specific examples of in vitro ADCC assays are described in Bruggemann et al., J. Exp. Med. 166:1351 (1987); Wilkinson et al., J. Immunol. Methods 258:183 (2001); Patel et al., J. Immunol. Methods 184:29 (1995). Alternatively, or additionally, ADCC activity of the antibody or bispecific binding protein of interest can be assessed in vivo, e.g., in an animal model such as that disclosed in Clynes et al., Proc. Natl. Acad. Sci. USA 95:652 (1998).

As used herein, the terms “condition” and “conditioning” refer to processes by which a patient is prepared for receipt of a transplant containing hematopoietic stem cells. Such procedures promote the engraftment of a hematopoietic stem cell transplant (for instance, as inferred from a sustained increase in the quantity of viable hematopoietic stem cells within a blood sample isolated from a patient following a conditioning procedure and subsequent hematopoietic stem cell transplantation. According to the methods described herein, a patient may be conditioned for hematopoietic stem cell transplant therapy by administration to the patient of a T cell mediated HSC cell depleting bispecific antibody, (e.g., anti-CD117 bispecific binding proteins or fragments thereof, capable of binding an antigen expressed by hematopoietic stem cells, such as CD117 (e.g., GNNK+ CD117) and an antigen expressed by a T cell, such as CD3). In certain embodiments, administration of bispecific binding proteins or fragments thereof, capable of binding an antigen on an HSC (e.g., CD117) and a T cell (e.g., CD3) to a patient in need of hematopoietic stem cell transplant therapy can promote the engraftment of a hematopoietic stem cell graft, for example, by selectively depleting endogenous hematopoietic stem cells, thereby creating a vacancy filled by an exogenous hematopoietic stem cell transplant.

Also provided are “conservative sequence modifications” of the sequences set forth in SEQ ID NOs described herein, i.e., nucleotide and amino acid sequence modifications which do not abrogate the binding of the antibody or bispecific binding protein encoded by the nucleotide sequence or containing the amino acid sequence, to the antigen. Such conservative sequence modifications include conservative nucleotide and amino acid substitutions, as well as, nucleotide and amino acid additions and deletions. For example, modifications can be introduced into SEQ ID NOs described herein by standard techniques known in the art, such as site-directed mutagenesis and PCR-mediated mutagenesis. Conservative sequence modifications include conservative amino acid substitutions, in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, a predicted nonessential amino acid residue in a bispecific antibody, e.g., an anti-CD117 antibody or anti-CD117 bispecific binding protein, is preferably replaced with another amino acid residue from the same side chain family. Methods of identifying nucleotide and amino acid conservative substitutions that do not eliminate antigen binding are well-known in the art (see, e.g., Brummell et al., Biochem. 32:1180-1187 (1993); Kobayashi et al. Protein Eng. 12(10):879-884 (1999); and Burks et al. Proc. Natl. Acad. Sci. USA 94:412-417 (1997)).

As used herein, the term “donor” refers to a human or animal from which one or more cells are isolated prior to administration of the cells, or progeny thereof, into a recipient. The one or more cells may be, for example, a population of hematopoietic stem cells.

As used herein, the term “diabody” refers to a bivalent antibody containing two polypeptide chains, in which each polypeptide chain includes V_{H} and V_{L} domains joined by a linker that is too short (e.g., a linker composed of five amino acids) to allow for intramolecular association of V_{H} and V_{L} domains on the same peptide chain. This configuration forces each domain to pair with a complementary domain on another polypeptide chain so as to form a homodimeric structure. Accordingly, the term “triabody” refers to trivalent antibodies containing three peptide chains, each of which contains one V_{H} domain and one V_{L} domain joined by a linker that is exceedingly short (e.g., a linker composed of 1-2 amino acids) to permit intramolecular association of V_{H} and V_{L} domains within the same peptide chain. In order to fold into their native structures, peptides configured in this way typically trimerize so as to position the V_{H} and V_{L}domains of neighboring peptide chains spatially proximal to one another (see, for example, Holliger et al., Proc. Natl. Acad. Sci. USA 90:6444-48, 1993).

As used herein, the term “endogenous” describes a substance, such as a molecule, cell, tissue, or organ (e.g., a hematopoietic stem cell or a cell of hematopoietic lineage, such as a megakaryocyte, thrombocyte, platelet, erythrocyte, mast cell, myeoblast, basophil, neutrophil, eosinophil, microglial cell, granulocyte, monocyte, osteoclast, antigen-presenting cell, macrophage, dendritic cell, natural killer cell, T-lymphocyte, or B-lymphocyte) that is found naturally in a particular organism, such as a human patient.

As used herein, the term “engraftment potential” is used to refer to the ability of hematopoietic stem and progenitor cells to repopulate a tissue, whether such cells are naturally circulating or are provided by transplantation. The term encompasses all events surrounding or leading up to engraftment, such as tissue homing of cells and colonization of cells within the tissue of interest. The engraftment efficiency or rate of engraftment can be evaluated or quantified using any clinically acceptable parameter as known to those of skill in the art and can include, for example, assessment of competitive repopulating units (CRU); incorporation or expression of a marker in tissue(s) into which stem cells have homed, colonized, or become engrafted; or by evaluation of the progress of a subject through disease progression, survival of hematopoietic stem and progenitor cells, or survival of a recipient. Engraftment can also be determined by measuring white blood cell counts in peripheral blood during a post-transplant period. Engraftment can also be assessed by measuring recovery of marrow cells by donor cells in a bone marrow aspirate sample.

As used herein, the term “exogenous” describes a substance, such as a molecule, cell, tissue, or organ (e.g., a hematopoietic stem cell or a cell of hematopoietic lineage, such as a megakaryocyte, thrombocyte, platelet, erythrocyte, mast cell, myeoblast, basophil, neutrophil, eosinophil, microglial cell, granulocyte, monocyte, osteoclast, antigen-presenting cell, macrophage, dendritic cell, natural killer cell, T-lymphocyte, or B-lymphocyte) that is not found naturally in a particular organism, such as a human patient. Exogenous substances include those that are provided from an external source to an organism or to cultured matter extracted therefrom.

As used herein, the term “framework region” or “FW region” includes amino acid residues that are adjacent to the CDRs of an antibody or antigen-binding fragment thereof. FW region residues may be present in, for example, human antibodies, humanized antibodies, monoclonal antibodies, antibody fragments, Fab fragments, single chain antibody fragments, scFv fragments, antibody domains, and bispecific binding proteins or fragments thereof, among others.

As used herein, the term “hematopoietic stem cells” (“HSCs”) refers to immature blood cells having the capacity to self-renew and to differentiate into mature blood cells containing diverse lineages including but not limited to granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). Such cells may include CD34+ cells. CD34+ cells are immature cells that express the CD34 cell surface marker. In humans, CD34+ cells are believed to include a subpopulation of cells with the stem cell properties defined above, whereas in mice, HSCs are CD34−. In addition, HSCs also refer to long term repopulating HSCs (LT-HSC) and short term repopulating HSCs (ST-HSC). LT-HSCs and ST-HSCs are differentiated, based on functional potential and on cell surface marker expression. For example, human HSCs are CD34+, CD38−, CD45RA−, CD90+, CD49F+, and lin− (negative for mature lineage markers including CD2, CD3, CD4, CD7, CD8, CD10, CD11B, CD19, CD20, CD56, CD235A). In mice, bone marrow LT-HSCs are CD34−, SCA-1+, C-kit+, CD135−, Slamfl/CD150+, CD48−, and lin−(negative for mature lineage markers including Ter119, CD11b, Gr1, CD3, CD4, CD8, B220, IL7ra), whereas ST-HSCs are CD34+, SCA-1+, C-kit+, CD135−, Slamfl/CD150+, and lin− (negative for mature lineage markers including Ter119, CD11b, Gr1, CD3, CD4, CD8, B220, IL7ra). In addition, ST-HSCs are less quiescent and more proliferative than LT-HSCs under homeostatic conditions. However, LT-HSC have greater self renewal potential (i.e., they survive throughout adulthood, and can be serially transplanted through successive recipients), whereas ST-HSCs have limited self renewal (i.e., they survive for only a limited period of time, and do not possess serial transplantation potential). Any of these HSCs can be used in the methods described herein. ST-HSCs are particularly useful because they are highly proliferative and thus, can more quickly give rise to differentiated progeny.

As used herein, the term “hematopoietic stem cell functional potential” refers to the functional properties of hematopoietic stem cells which include 1) multi-potency (which refers to the ability to differentiate into multiple different blood lineages including, but not limited to, granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells), 2) self-renewal (which refers to the ability of hematopoietic stem cells to give rise to daughter cells that have equivalent potential as the mother cell, and further that this ability can repeatedly occur throughout the lifetime of an individual without exhaustion), and 3) the ability of hematopoietic stem cells or progeny thereof to be reintroduced into a transplant recipient whereupon they home to the hematopoietic stem cell niche and re-establish productive and sustained hematopoiesis.

As used herein, the term “human antibody” is intended to include antibodies having variable and constant regions derived from human germline immunoglobulin sequences. A human antibody may include amino acid residues not encoded by human germline immunoglobulin sequences (e.g., mutations introduced by random or site-specific mutagenesis in vitro or during gene rearrangement or by somatic mutation in vivo). However, the term “human antibody”, as used herein, is not intended to include antibodies in which CDR sequences derived from the germline of another mammalian species, such as a mouse, have been grafted onto human framework sequences. A human antibody can be produced in a human cell (for example, by recombinant expression) or by a non-human animal or a prokaryotic or eukaryotic cell that is capable of expressing functionally rearranged human immunoglobulin (such as heavy chain and/or light chain) genes. When a human antibody is a single chain antibody, it can include a linker peptide that is not found in native human antibodies. For example, an Fv can contain a linker peptide, such as two to about eight glycine or other amino acid residues, which connects the variable region of the heavy chain and the variable region of the light chain. Such linker peptides are considered to be of human origin. Human antibodies can be made by a variety of methods known in the art including phage display methods using antibody libraries derived from human immunoglobulin sequences. Human antibodies can also be produced using transgenic mice that are incapable of expressing functional endogenous immunoglobulins, but which can express human immunoglobulin genes (see, for example, PCT Publication Nos. WO 1998/24893; WO 1992/01047; WO 1996/34096; WO 1996/33735; U.S. Pat. Nos. 5,413,923; 5,625,126; 5,633,425; 5,569,825; 5,661,016; 5,545,806; 5,814,318; 5,885,793; 5,916,771; and 5,939,598).

As used herein, patients that are “in need of” a hematopoietic stem cell transplant include patients that exhibit a defect or deficiency in one or more blood cell types, as well as patients having a stem cell disorder, autoimmune disease, cancer, or other pathology described herein. Hematopoietic stem cells generally exhibit 1) multi-potency, and can thus differentiate into multiple different blood lineages including, but not limited to, granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells), 2) self-renewal, and can thus give rise to daughter cells that have equivalent potential as the mother cell, and 3) the ability to be reintroduced into a transplant recipient whereupon they home to the hematopoietic stem cell niche and re-establish productive and sustained hematopoiesis. Hematopoietic stem cells can thus be administered to a patient defective or deficient in one or more cell types of the hematopoietic lineage in order to re-constitute the defective or deficient population of cells in vivo. For example, the patient may be suffering from cancer, and the deficiency may be caused by administration of a chemotherapeutic agent or other medicament that depletes, either selectively or non-specifically, the cancerous cell population. Additionally or alternatively, the patient may be suffering from a hemoglobinopathy (e.g., a non-malignant hemoglobinopathy), such as sickle cell anemia, thalassemia, Fanconi anemia, aplastic anemia, and Wiskott-Aldrich syndrome. The subject may be one that is suffering from adenosine deaminase severe combined immunodeficiency (ADA SCID), HIV/AIDS, metachromatic leukodystrophy, Diamond-Blackfan anemia, and Schwachman-Diamond syndrome. The subject may have or be affected by an inherited blood disorder (e.g., sickle cell anemia) or an autoimmune disorder. Additionally or alternatively, the subject may have or be affected by a malignancy, such as neuroblastoma or a hematologic cancer. For instance, the subject may have a leukemia, lymphoma, or myeloma. In some embodiments, the subject has acute myeloid leukemia, acute lymphoid leukemia, chronic myeloid leukemia, chronic lymphoid leukemia, multiple myeloma, diffuse large B-cell lymphoma, or non-Hodgkin's lymphoma. In some embodiments, the subject has myelodysplastic syndrome. In some embodiments, the subject has an autoimmune disease, such as scleroderma, multiple sclerosis, ulcerative colitis, Crohn's disease, Type 1 diabetes, or another autoimmune pathology described herein. In some embodiments, the subject is in need of chimeric antigen receptor T-cell (CART) therapy. In some embodiments, the subject has or is otherwise affected by a metabolic storage disorder. The subject may suffer or otherwise be affected by a metabolic disorder selected from the group consisting of glycogen storage diseases, mucopolysaccharidoses, Gaucher's Disease, Hurlers Disease, sphingolipidoses, metachromatic leukodystrophy, or any other diseases or disorders which may benefit from the treatments and therapies disclosed herein and including, without limitation, severe combined immunodeficiency, Wiscott-Aldrich syndrome, hyper immunoglobulin M (IgM) syndrome, Chediak-Higashi disease, hereditary lymphohistiocytosis, osteopetrosis, osteogenesis imperfecta, storage diseases, thalassemia major, sickle cell disease, systemic sclerosis, systemic lupus erythematosus, multiple sclerosis, juvenile rheumatoid arthritis and those diseases, or disorders described in “Bone Marrow Transplantation for Non-Malignant Disease,” ASH Education Book, 1:319-338 (2000), the disclosure of which is incorporated herein by reference in its entirety as it pertains to pathologies that may be treated by administration of hematopoietic stem cell transplant therapy. Additionally or alternatively, a patient “in need of” a hematopoietic stem cell transplant may one that is or is not suffering from one of the foregoing pathologies, but nonetheless exhibits a reduced level (e.g., as compared to that of an otherwise healthy subject) of one or more endogenous cell types within the hematopoietic lineage, such as megakaryocytes, thrombocytes, platelets, erythrocytes, mast cells, myeoblasts, basophils, neutrophils, eosinophils, microglia, granulocytes, monocytes, osteoclasts, antigen-presenting cells, macrophages, dendritic cells, natural killer cells, T-lymphocytes, and B-lymphocytes. One of skill in the art can readily determine whether one's level of one or more of the foregoing cell types, or other blood cell type, is reduced with respect to an otherwise healthy subject, for instance, by way of flow cytometry and fluorescence activated cell sorting (FACS) methods, among other procedures, known in the art.

As used herein, the term “recipient” refers to a patient that receives a transplant, such as a transplant containing a population of hematopoietic stem cells. The transplanted cells administered to a recipient may be, e.g., autologous, syngeneic, or allogeneic cells.

As used herein, the term “sample” refers to a specimen (e.g., blood, blood component (e.g., serum or plasma), urine, saliva, amniotic fluid, cerebrospinal fluid, tissue (e.g., placental or dermal), pancreatic fluid, chorionic villus sample, and cells) taken from a subject.

As used herein, the term “scFv” refers to a single chain Fv antibody in which the variable domains of the heavy chain and the light chain from an antibody have been joined to form one chain. scFv fragments contain a single polypeptide chain that includes the variable region of an antibody (or bispecific binding protein as described herein) light chain (V_{L}) (e.g., CDR-L1, CDR-L2, and/or CDR-L3) and the variable region of an antibody (or bispecific binding protein as described herein) heavy chain (V_{H}) (e.g., CDR-H1, CDR-H2, and/or CDR-H3) separated by a linker. The linker that joins the V_{L} and V_{H} regions of a scFv fragment can be a peptide linker composed of proteinogenic amino acids. Alternative linkers can be used to so as to increase the resistance of the scFv fragment to proteolytic degradation (for example, linkers containing D-amino acids), in order to enhance the solubility of the scFv fragment (for example, hydrophilic linkers such as polyethylene glycol-containing linkers or polypeptides containing repeating glycine and serine residues), to improve the biophysical stability of the molecule (for example, a linker containing cysteine residues that form intramolecular or intermolecular disulfide bonds), or to attenuate the immunogenicity of the scFv fragment (for example, linkers containing glycosylation sites). It will also be understood by one of ordinary skill in the art that the variable regions of the scFv molecules described herein can be modified such that they vary in amino acid sequence from the antibody molecule from which they were derived. For example, nucleotide or amino acid substitutions leading to conservative substitutions or changes at amino acid residues can be made (e.g., in CDR and/or framework residues) so as to preserve or enhance the ability of the scFv to bind to the antigen recognized by the corresponding antibody or bispecific binding protein.

As used herein, the terms “subject” and “patient” refer to an organism, such as a human, that receives treatment for a particular disease or condition as described herein. For instance, a patient, such as a human patient, may receive treatment prior to hematopoietic stem cell transplant therapy in order to promote the engraftment of exogenous hematopoietic stem cells.

As used herein, the phrase “substantially cleared from the blood” refers to a point in time following administration of a therapeutic agent (such as an anti-CD117 bispecific antibody, or antigen-binding fragment thereof) to a patient when the concentration of the therapeutic agent in a blood sample isolated from the patient is such that the therapeutic agent is not detectable by conventional means (for instance, such that the therapeutic agent is not detectable above the noise threshold of the device or assay used to detect the therapeutic agent). A variety of techniques known in the art can be used to detect antibodies, antibody fragments, bispecific antibodies and antigen-binding fragments thereof, such as ELISA-based detection assays known in the art or described herein. Additional assays that can be used to detect antibodies, or antibody fragments, bispecific antibodies and antigen-binding fragments thereof, include immunoprecipitation techniques and immunoblot assays, among others known in the art.

As used herein, the phrase “stem cell disorder” broadly refers to any disease, disorder, or condition that may be treated or cured by conditioning a subject's target tissues, and/or by ablating an endogenous stem cell population in a target tissue (e.g., ablating an endogenous hematopoietic stem or progenitor cell population from a subject's bone marrow tissue) and/or by engrafting or transplanting stem cells in a subject's target tissues. For example, Type I diabetes has been shown to be cured by hematopoietic stem cell transplant and may benefit from conditioning in accordance with the compositions and methods described herein. Additional disorders that can be treated using the compositions and methods described herein include, without limitation, sickle cell anemia, thalassemias, Fanconi anemia, aplastic anemia, Wiskott-Aldrich syndrome, ADA SCID, HIV/AIDS, metachromatic leukodystrophy, Diamond-Blackfan anemia, and Schwachman-Diamond syndrome. Additional diseases that may be treated using the patient conditioning and/or hematopoietic stem cell transplant methods described herein include inherited blood disorders (e.g., sickle cell anemia) and autoimmune disorders, such as scleroderma, multiple sclerosis, ulcerative colitis, and Crohn's disease. Additional diseases that may be treated using the conditioning and/or transplantation methods described herein include a malignancy, such as a neuroblastoma or a hematologic cancer, such as leukemia, lymphoma, and myeloma. For instance, the cancer may be acute myeloid leukemia, acute lymphoid leukemia, chronic myeloid leukemia, chronic lymphoid leukemia, multiple myeloma, diffuse large B-cell lymphoma, or non-Hodgkin's lymphoma. Additional diseases treatable using the conditioning and/or transplantation methods described herein include myelodysplastic syndrome. In some embodiments, the subject has or is otherwise affected by a metabolic storage disorder. For example, the subject may suffer or otherwise be affected by a metabolic disorder selected from the group consisting of glycogen storage diseases, mucopolysaccharidoses, Gaucher's Disease, Hurlers Disease, sphingolipidoses, metachromatic leukodystrophy, or any other diseases or disorders which may benefit from the treatments and therapies disclosed herein and including, without limitation, severe combined immunodeficiency, Wiscott-Aldrich syndrome, hyper immunoglobulin M (IgM) syndrome, Chediak-Higashi disease, hereditary lymphohistiocytosis, osteopetrosis, osteogenesis imperfecta, storage diseases, thalassemia major, sickle cell disease, systemic sclerosis, systemic lupus erythematosus, multiple sclerosis, juvenile rheumatoid arthritis and those diseases, or disorders described in “Bone Marrow Transplantation for Non-Malignant Disease,” ASH Education Book, 1:319-338 (2000), the disclosure of which is incorporated herein by reference in its entirety as it pertains to pathologies that may be treated by administration of hematopoietic stem cell transplant therapy.

As used herein, the term “transfection” refers to any of a wide variety of techniques commonly used for the introduction of exogenous DNA into a prokaryotic or eukaryotic host cell, such as electroporation, lipofection, calcium-phosphate precipitation, DEAE-dextran transfection and the like.

As used herein, the terms “treat” or “treatment” refers to reducing the severity and/or frequency of disease symptoms, eliminating disease symptoms and/or the underlying cause of said symptoms, reducing the frequency or likelihood of disease symptoms and/or their underlying cause, and improving or remediating damage caused, directly or indirectly, by disease. Beneficial or desired clinical results include, but are not limited to, promoting the engraftment of exogenous hematopoietic cells in a patient following antibody conditioning therapy as described herein and subsequent hematopoietic stem cell transplant therapy. Additional beneficial results include an increase in the cell count or relative concentration of hematopoietic stem cells in a patient in need of a hematopoietic stem cell transplant following conditioning therapy and subsequent administration of an exogenous hematopoietic stem cell graft to the patient. Beneficial results of therapy described herein may also include an increase in the cell count or relative concentration of one or more cells of hematopoietic lineage, such as a megakaryocyte, thrombocyte, platelet, erythrocyte, mast cell, myeoblast, basophil, neutrophil, eosinophil, microglial cell, granulocyte, monocyte, osteoclast, antigen-presenting cell, macrophage, dendritic cell, natural killer cell, T-lymphocyte, or B-lymphocyte, following conditioning therapy and subsequent hematopoietic stem cell transplant therapy. Additional beneficial results may include the reduction in quantity of a disease-causing cell population, such as a population of cancer cells (e.g., CD117+ leukemic cells) or autoimmune cells (e.g., CD117+ autoimmune lymphocytes, such as a CD117+T-cell that expresses a T-cell receptor that cross-reacts with a self antigen). Insofar as the methods of the present invention are directed to preventing disorders, it is understood that the term “prevent” does not require that the disease state be completely thwarted. Rather, as used herein, the term preventing refers to the ability of the skilled artisan to identify a population that is susceptible to disorders, such that administration of the compounds of the present invention may occur prior to onset of a disease. The term does not imply that the disease state is completely avoided.

As used herein, the terms “variant” and “derivative” are used interchangeably and refer to naturally-occurring, synthetic, and semi-synthetic analogues of a compound, peptide, protein, or other substance described herein. A variant or derivative of a compound, peptide, protein, or other substance described herein may retain or improve upon the biological activity of the original material.

As used herein, the term “vector” includes a nucleic acid vector, such as a plasmid, a DNA vector, a plasmid, a RNA vector, virus, or other suitable replicon. Expression vectors described herein may contain a polynucleotide sequence as well as, for example, additional sequence elements used for the expression of proteins and/or the integration of these polynucleotide sequences into the genome of a mammalian cell. Certain vectors that can be used for the expression of bispecific antibodies and bispecific antibody fragments of the invention include plasmids that contain regulatory sequences, such as promoter and enhancer regions, which direct gene transcription. Other useful vectors for expression of bispecific antibodies and bispecific antibody fragments contain polynucleotide sequences that enhance the rate of translation of these genes or improve the stability or nuclear export of the mRNA that results from gene transcription. These sequence elements may include, for example, 5′ and 3′ untranslated regions and a polyadenylation signal site in order to direct efficient transcription of the gene carried on the expression vector. The expression vectors described herein may also contain a polynucleotide encoding a marker for selection of cells that contain such a vector. Examples of a suitable marker include genes that encode resistance to antibiotics, such as ampicillin, chloramphenicol, kanamycin, and nourseothricin.

### Bispecific Binding Proteins Targeting T-Cell Antigen and an HSC Antigen

The compositions and methods described herein are based on T cell mediated depletion of target cells, particularly HSCs as target cells which are depleted for conditioning. One advantage to the disclosure herein is the ability of the bispecific binding proteins, eg., an anti-CD3/anti-HSC bispecific antibody, to deplete HSCs using immune-mediated cytotoxicity and without the need, or a reduced need, for cytotoxic agents or therapies that cause general cell depletion, e.g., chemotherapy or irradiation. This targeted approach focuses on cells expressing antigens associated with the target cell population, e.g., HSCs, and minimizes the impact on cells that are not being targeted. Further, cell depletion is accomplished by directing T cells to the target cells using the bispecific proteins disclosed herein.

As used herein, the term “anti-hematopoietic cell antibody” or “anti-HC antibody” refers to an antibody that specifically binds an antigen expressed by hematopoietic stem cells, such as CD117 (e.g., GNNK+ CD117). A bispecific antibody, or a bispecific antigen-binding region thereof, may comprise a first binding moiety that is derived from an anti-HC antibody, e.g., a heavy and light chain combination specific for CD117.

Compositions and methods, including bispecifics, disclosed herein can be used to target cells expressing any target-specific antigen. In certain embodiments, compositions and methods disclosed herein are specific for antigens expressed on human HSCs, antigens such as CD7, CDw12, CD13, CD15, CD19, CD21, CD22, CD29, CD30, CD33, CD34, CD36, CD38, CD40, CD41, CD42a, CD42b, CD42c, CD42d, CD43, CD48, CD49b, CD49d, CD49e, CD49f, CD50, CD53, CD55, CD64a, CD68, CD71, CD72, CD73, CD81, CD82, CD85A, CD85K, CD90, CD99, CD104, CD105, CD109, CD110, CD111, CD112, CD114, CD115, CD117, CD123, CD124, CD126, CD127, CD130, CD131, CD133, CD135, CD138, CD151, CD157, CD162, CD164, CD168, CD172a, CD173, CD174, CD175, CD175s, CD176, CD183, CD191, CD200, CD201, CD205, CD217, CD220, CD221, CD222, CD223, CD224, CD225, CD226, CD227, CD228, CD229, CD230, CD235a, CD235b, CD236, CD236R, CD238, CD240, CD242, CD243, CD277, CD292, CDw293, CD295, CD298, CD309, CD318, CD324, CD325, CD338, CD344, CD349 and CD350. In certain embodiments, the targeted cells comprise human hematopoietic stem cells expressing one or more markers that may be targeted, such antigens include CD11a, CD18, CD37, CD47, CD52, CD58, CD62L, CD69, CD74, CD97, CD103, CD132, CD156a, CD179a, CD179b, CD184, CD232, CD244, CD252, CD302, CD305, CD317 or CD361.

In certain embodiments, the targeted cells are human hematopoietic stem cells expressing one or more markers that may be targeted by the anti-CD3 bispecific antibody disclosed herein, wherein the marker is CD7, CDw12, CD13, CD15, CD19, CD21, CD22, CD29, CD30, CD33, CD34, CD36, CD38, CD40, CD41, CD42a, CD42b, CD42c, CD42d, CD43, CD48, CD49b, CD49d, CD49e, CD49f, CD50, CD53, CD55, CD64a, CD68, CD71, CD72, CD73, CD81, CD82, CD85A, CD85K, CD90, CD99, CD104, CD105, CD109, CD110, CD111, CD112, CD114, CD115, CD117, CD123, CD124, CD126, CD127, CD130, CD131, CD133, CD135, CD138, CD151, CD157, CD162, CD164, CD168, CD172a, CD173, CD174, CD175, CD175s, CD176, CD183, CD191, CD200, CD201, CD205, CD217, CD220, CD221, CD222, CD223, CD224, CD225, CD226, CD227, CD228, CD229, CD230, CD235a, CD235b, CD236, CD236R, CD238, CD240, CD242, CD243, CD277, CD292, CDw293, CD295, CD298, CD309, CD318, CD324, CD325, CD338, CD344, CD349, or CD350. The present disclosure provides bispecific antibodies comprising a first binding domain which binds to an antigen expressed on the surface of a hematopoietic stem cell, and a second binding domain which binds to human CD3 on the surface of a T cell. In some embodiments of the present disclosure, the bispecific antibody binds to human CD3 epsilon.

In certain embodiments, the anti-CD3 binding domain comprises antigen binding regions (variable regions or CDRs) from anti-CD3 antibodies described in U.S. Pat. Nos. 10,851,170, 10,933,132, 10,781,264, 10,738,130, and WO 2008/119567, each of which are hereby incorporated herein by reference in their entirety.

In some embodiments, the anti-CD3 binding domain of the bispecific antibody comprises heavy chain and a light chain variable regions as described in Table 4. In one embodiment, the anti-CD3 binding domain of the bispecific antibody comprises a heavy chain comprising a CDR1, CDR2 and CDR3, and a light chain variable region comprising a CDR1, CDR2 and CDR3 as described in Table 4.

In other embodiments, an anti-CD3/anti-HC bispecific antibody, or bispecific antigen-binding fragment thereof, comprises an anti-CD3 binding moiety comprising a light and/or heavy chain variable region that comprises an amino acid sequence having at least 95% identity to an anti-CD3 light and/or heavy chain variable region sequence described in Table 4, e.g., at least 95%, 96%, 97%, 98%, 99%, or 100% identity. In certain embodiments, an anti-CD3/anti-HC bispecific antibody, or bispecific antigen-binding fragment thereof, comprises a modified light or heavy chain variable region comprising a light and/or heavy chain variable domain of an anti-CD3 antibody described in Table 4, or a variant thereof, which variant (i) differs from the anti-CD3 antigen binding region in 1, 2, 3, 4 or 5 amino acids substitutions, additions or deletions; (ii) differs from the anti-CD3 antigen binding region in at most 5, 4, 3, 2, or 1 amino acids substitutions, additions or deletions; (iii) differs from the anti-CD3 antigen binding region in 1-5, 1-3, 1-2, 2-5 or 3-5 amino acids substitutions, additions or deletions and/or (iv) comprises an amino acid sequence that is at least about 75%, 80%, 85%, 90%, 95%, 96%, 97%, 98% or 99% identical to the anti-CD3 antigen binding region, wherein in any of (i)-(iv), an amino acid substitution may be a conservative amino acid substitution or a non-conservative amino acid substitution; and wherein the modified light and/or heavy chain variable region can have an enhanced biological activity relative to the light and/or heavy chain variable region of the anti-CD3 antibody, while retaining the CD3 binding specificity of the bispecific antibody.

In other embodiments, the anti-CD3 antigen binding region of the bispecific disclosed herein comprises a variable light chain (VL) region comprising CDR-L1, CDR-L2 and CDR-L3 selected from the following sequences described in Table 4: (a) CDR-L1 as depicted in SEQ ID NO: 60, CDR-L2 as depicted in SEQ ID NO: 61, and CDR-L3 as depicted in SEQ ID NO: 62; (b) CDR-L1 as depicted in SEQ ID NO: 108, CDR-L2 as depicted in SEQ ID NO: 109, and CDR-L3 as depicted in SEQ ID NO: 110; and (c) CDR-L1 as depicted in SEQ ID NO: 129, CDR-L2 as depicted in SEQ ID NO: 130, and CDR-L3 as depicted in SEQ ID NO: 131.

In other embodiments, the anti-CD3 antigen binding region of the bispecific disclosed herein comprises a variable heavy chain (VH) region comprising CDR-H1, CDR-H2 and CDR-H3 selected from the following sequences described in Table 4: (a) CDR-H1 as depicted in SEQ ID NO: 51, CDR-H2 as depicted in SEQ ID NO: 52, and CDR-H3 as depicted in SEQ ID NO: 53; (b) CDR-H1 as depicted in SEQ ID NO: 63, CDR-H2 as depicted in SEQ ID NO: 64, and CDR-H3 as depicted in SEQ ID NO: 65; (c) CDR-H1 as depicted in SEQ ID NO: 72, CDR-H2 as depicted in SEQ ID NO: 73, and CDR-H3 as depicted in SEQ ID NO: 74; (d) CDR-H1 as depicted in SEQ ID NO: 81, CDR-H2 as depicted in SEQ ID NO: 82, and CDR-H3 as depicted in SEQ ID NO: 83; (e) CDR-H1 as depicted in SEQ ID NO: 90, CDR-H2 as depicted in SEQ ID NO: 91, and CDR-H3 as depicted in SEQ ID NO: 92; (f) CDR-H1 as depicted in SEQ ID NO: 99, CDR-H2 as depicted in SEQ ID NO: 100, and CDR-H3 as depicted in SEQ ID NO: 101; (g) CDR-H1 as depicted in SEQ ID NO: 111, CDR-H2 as depicted in SEQ ID NO: 112, and CDR-H3 as depicted in SEQ ID NO: 113; (h) CDR-H1 as depicted in SEQ ID NO: 120, CDR-H2 as depicted in SEQ ID NO: 121, and CDR-H3 as depicted in SEQ ID NO: 122; (i) CDR-H1 as depicted in SEQ ID NO: 132, CDR-H2 as depicted in SEQ ID NO: 133, and CDR-H3 as depicted in SEQ ID NO: 134; and (j) CDR-H1 as depicted in SEQ ID NO: 141, CDR-H2 as depicted in SEQ ID NO: 142, and CDR-H3 as depicted in SEQ ID NO: 143.

In further embodiments, the anti-CD3 antigen binding region of the bispecific disclosed herein comprises a VL region selected from the group consisting of a VL region as depicted in SEQ ID NOs: 67, 69, 115, 117, 136 or 138 of Table 4.

In other embodiments, the anti-CD3 antigen binding region of the bispecific disclosed herein comprises a VH region selected from the group consisting of a VH region as depicted in SEQ ID NOs: 54, 56, 66, 68, 75, 77, 84, 86, 93, 95, 102, 104, 114, 116, 123, 125, 135, 137, 144 or 146 of Table 4.

In certain other embodiments, the anti-CD3 antigen binding region of the bispecific disclosed herein comprises a VL region and a VH region selected from the group consisting of the following sequences described in Table 4: (a) a VL region as depicted in SEQ ID NO: 55 or 57, and a VH region as depicted in SEQ ID NO: 54 or 56; (b) a VL region as depicted in SEQ ID NO: 67 or 69, and a VH region as depicted in SEQ ID NO: 66 or 68; (c) a VL region as depicted in SEQ ID NO: 76 or 78, and a VH region as depicted in SEQ ID NO: 75 or 77; (d) a VL region as depicted in SEQ ID NO: 85 or 87, and a VH region as depicted in SEQ ID NO: 84 or 86; (e) a VL region as depicted in SEQ ID NO: 94 or 96, and a VH region as depicted in SEQ ID NO: 93 or 95; (f) a VL region as depicted in SEQ ID NO: 103 or 105, and a VH region as depicted in SEQ ID NO: 102 or 104; (g) a VL region as depicted in SEQ ID NO: 115 or 117, and a VH region as depicted in SEQ ID NO: 114 or 116; (h) a VL region as depicted in SEQ ID NO: 124 or 126, and a VH region as depicted in SEQ ID NO: 123 or 125; (i) a VL region as depicted in SEQ ID NO: 136 or 138, and a VH region as depicted in SEQ ID NO: 135 or 137; and (j) a VL region as depicted in SEQ ID NO: 145 or 147, and a VH region as depicted in SEQ ID NO: 144 or 146.

In other embodiments of the present disclosure, the anti-CD3 binding domain comprises a pair of VH regions and VL regions disclosed herein (or variable regions having CDRs disclosed herein) in the format of a single chain antibody (scFv). The VH and VL regions are arranged in the order VH-VL or VL-VH. In one embodiment, the VH-region is positioned N-terminally of a linker sequence, and the VL-region is positioned C-terminally of the linker sequence.

In other embodiments of the present disclosure, the anti-CD3 antigen binding region of the bispecific disclosed herein comprises an amino acid sequence selected from the group consisting of SEQ ID NOs: 58, 59, 70, 71, 79, 80, 88, 89, 97, 98, 106, 107, 118, 119, 127, 128, 139, 140, 148 or 149 of Table 4.

### Anti-CD117/Anti-CD3 Bispecific Binding Proteins

The present disclosure is based in part on the discovery that bispecific binding proteins, or antigen-binding fragments thereof, capable of binding a T cell specific antigen (e.g., CD3) and CD117, such as GNNK+ CD117, can be used as therapeutic agents to (i) treat cancers (such as acute myelogenous leukemia or myelodysplastic syndrome) and autoimmune diseases characterized by CD117+ cells and (ii) promote the engraftment of transplanted hematopoietic stem cells in a patient in need of transplant therapy. These therapeutic activities can be caused, for instance, by the binding of anti-CD117 bispecific antibodies, or antigen-binding fragments thereof, to CD117 (e.g., GNNK+ CD117) expressed on the surface of a cell, such as a cancer cell, autoimmune cell, or hematopoietic stem cell and subsequently inducing cell death. The depletion of endogenous hematopoietic stem cells can provide a niche toward which transplanted hematopoietic stem cells can home, and subsequently establish productive hematopoiesis. In this way, transplanted hematopoietic stem cells may successfully engraft in a patient, such as human patient suffering from a stem cell disorder described herein.

Accordingly, provided herein are bispecific binding polypeptides, or fragments thereof, that bind to both CD117 and CD3. Also provided herein are isolated nucleic acids (polynucleotides), such as complementary DNA (cDNA), encoding such bispecific binding polypeptides or fragments thereof. Further provided are vectors (e.g., expression vectors) comprising nucleic acids (polynucleotides) or vectors (e.g., expression vectors) encoding such bispecific binding molecules or fragments thereof. Also provided herein are methods of making such bispecific binding molecules, cells, and vectors. In other embodiments, provided herein are methods and uses for treating various blood diseases, metabolic disorders, cancers, and autoimmune diseases, among others, using the bispecific binding polypeptides, nucleic acids, and/or vectors, described herein. Additionally, related compositions (e.g., pharmaceutical compositions), kits, and diagnostic methods are also provided herein.

In certain embodiments, provided herein are bispecific binding polypeptides, or fragments thereof, that specifically bind to CD117 and to CD3, and invoke T cell cytotoxicity for treating various diseases, including, but not limited to blood diseases, stem cell diseases, cancer and immune disorders. Without being bound by any theory, it is believed that the bispecific binding molecules described herein not only bind tumors to T cells, they also cross-link CD3 on T cells and initiate the activation cascade, and, this way, T cell receptor (TCR)-based cytotoxicity is redirected to desired tumor targets, bypassing major histocompatibility complex (MHC) restrictions.

Bispecific binding polypeptides, or fragments thereof capable of binding human CD117 (also referred to as c-Kit, mRNA NCBI Reference Sequence: NM_000222.2, Protein NCBI Reference Sequence: NP_000213.1), including those capable of binding GNNK+ CD117, can be used in conjunction with the compositions and methods described herein in order to condition a patient for hematopoietic stem cell transplant therapy. Polymorphisms affecting the coding region or extracellular domain of CD117 in a significant percentage of the population are not currently well-known in non-oncology indications. There are at least four isoforms of CD117 that have been identified, with the potential of additional isoforms expressed in tumor cells. Two of the CD117 isoforms are located on the intracellular domain of the protein, and two are present in the external juxtamembrane region. The two extracellular isoforms, GNNK+ and GNNK−, differ in the presence (GNNK+) or absence (GNNK−) of a 4 amino acid sequence. These isoforms are reported to have the same affinity for the ligand (SCF), but ligand binding to the GNNK− isoform was reported to increase internalization and degradation. The GNNK+ isoform can be used as an immunogen in order to generate antibodies capable of binding CD117, as antibodies generated against this isoform will be inclusive of the GNNK+ and GNNK− proteins.

CD3 is a T cell co-receptor comprised of a gamma chain, a delta chain, and two epsilon chains. In a specific embodiment, CD3 is a human CD3. GenBank™ accession number NM_000073.2 (SEQ ID NO:31) provides an exemplary human CD3 gamma nucleic acid sequence. GenBank™ accession number NP_000064.1 (SEQ ID NO: 32) provides an exemplary human CD3 gamma amino acid sequence. GenBank™ accession number NM_000732.4 (SEQ ID NO: 33) provides an exemplary human CD3 delta nucleic acid sequence. GenBank™ accession number NP_000723.1 (SEQ ID NO:34) provides an exemplary human CD3 delta amino acid sequence. GenBank™ accession number NM_000733.3 (SEQ ID NO: 35) provides an exemplary human CD3 epsilon nucleic acid sequence. GenBank™ accession number NP_000724.1 (SEQ ID NO: 36) provides an exemplary human CD3 epsilon amino acid sequence. Also preferred in connection with the anti-CD117 bispecific binding proteins or fragments thereof of the present invention is a second binding domain which binds to human CD3 on the surface of a T cell comprising a VL region as depicted in SEQ ID NO: 38 and a VH region as depicted in SEQ ID NO: 37.

The immunoglobulin in the bispecific binding molecules of the invention can be, as non-limiting examples, a monoclonal antibody, a naked antibody, a chimeric antibody, a humanized antibody, or a human antibody.

In one embodiment, the anti-CD117 bispecific binding polypeptides, or fragments thereof, comprises a heavy chain variable region as set forth in the amino acid sequence of SEQ ID NO: 13, and a light chain variable region as set forth in the amino acid sequence of SEQ ID NO: 14.

In another embodiment, the anti-CD117 bispecific binding polypeptides, or fragments thereof, comprises the three CDR sequences of the heavy chain variable region (VH) amino acid sequence and the three CDR sequences of the light chain variable region (LH) amino acid sequence of Ab85.

In another embodiment, the anti-CD117 bispecific binding polypeptides, or fragments thereof, comprises the heavy chain variable region (VH) amino acid sequence and the light chain variable region (LH) amino acid sequence of Ab85.

The heavy chain variable region (VH) amino acid sequence provided below as SEQ ID NO: 13. The VH CDR amino acid sequences of Ab85 are underlined below and are as follows: NYWIG (VH CDR1; SEQ ID NO: 7); IINPRDSDTRYRPSFQG (VH CDR2; SEQ ID NO: 8); and HGRGYEGYEGAFDI (VH CDR3; SEQ ID NO: 9).

Ab85 VH sequence
(SEQ ID NO: 13)
EVQLVQSGAEVKKPGESLKISCKGSGYSFTNYWIG
WVRQMPGKGLEWMAIINPRDSDTRYRPSFQGQVTI
SADKSISTAYLQWSSLKASDTAMYYCARHGRGYEG
YEGAFDIWGQGTLVTVSS

The light chain variable region (VL) amino acid sequence of Ab85 is provided below as SEQ ID NO 14. The VL CDR amino acid sequences of Ab85 are underlined below and are as follows:

(VL CDR1; SEQ ID NO: 10)
RSSQGIRSDLG;
(VL CDR2; SEQ ID NO: 11
DASNLET;
and
(VL CDR3; SEQ ID NO: 12)
QQANGFPLT.

Ab85 VL sequence
(SEQ ID NO: 14)
DIQMTQSPSSLSASVGDRVTITCRSSQGIRSDLG
WYQQKPGKAPKLLIYDASNLETGVPSRFSGSGSG
TDFTLTISSLQPEDFATYYCQQANGFPLTFGGGT
KVEIK

Thus, in certain embodiments, the anti-CD117 bispecific binding polypeptides, or fragments thereof, comprises a heavy chain comprising a CDR set (CDR1, CDR2, and CDR3) as set forth in SEQ ID Nos: 7, 8, and 9, and a light chain comprising a CDR set as set forth in SEQ ID Nos: 10, 11, and 12.

In another embodiment, the anti-CD117 bispecific binding polypeptides, or fragments thereof, comprises the heavy chain variable region (VH) amino acid sequence and the light chain variable region (LH) amino acid sequence of Ab67.

The heavy chain variable region (VH) amino acid sequence of Ab67 is described in SEQ ID NO: 27.

(SEQ ID NO: 27; CDR domains are in bold)
EVQLVESGGGLVQPGGSLRLSCAASGFTFSDA
DMDWVRQAPGKGLEWVGRTRNKAGSYTTEYAA
SVKGRFTISRDDSKNSLYLQMNSLKTEDTAVY
YCAREPKYWIDFDLWGRGTLVTVSS

The VH CDR amino acid sequences of Ab67 are as follows: FTFSDADMD (VH CDR1; SEQ ID NO: 21); RTRNKAGSYTTEYAASVKG (VH CDR2; SEQ ID NO: 22); and AREPKYWIDFDL (VH CDR3; SEQ ID NO: 23).

The light chain variable region (VL) amino acid sequence of Ab67 is provided below as SEQ ID NO 28.

(SEQ ID NO: 28; CDR domains in bold)
DIQMTQSPSSLSASVGDRVTITCRASQSISSYLNW
YQQKPGKAPKLLIYAASSLQSGVPSRFSGSGSGTD
FTLTISSLQPEDFATYYCQQSYIAPYTFGGGTKVE
IK

The VL CDR amino acid sequences of Ab67 are underlined below and are as follows:

(VL CDR1; SEQ ID NO: 24)
RASQSISSYLN;
(VL CDR2; SEQ ID NO: 25)
AASSLQS;
and
(VL CDR3; SEQ ID NO: 26)
QQSYIAPYT.

In other embodiments, the anti-CD117 bispecific antibody, or fragment thereof, as disclosed herein, is capable of binding to a CD117 epitope, such as the epitope(s) described in WO 2020/219770, which is hereby incorporated herein by reference in its entirety. In yet other embodiments, the anti-CD117 bispecific antibody, or fragment thereof, as disclosed herein, is capable of binding to a CD117 epitope, such as the epitope(s) described in WO 2020/219748, which is hereby incorporated herein by reference in its entirety.

In certain embodiments of the anti-CD117 bispecific binding polypeptides, or fragments thereof, as disclosed herein comprises a scFv that binds to CD3, which comprises the VH and the VL of a CD3-specific antibody known in the art, such as, for example, huOKT3 (see, for example, Adair et al., 1994, Hum Antibodies Hybridomas 5:41-47), YTH12.5 (see, for example Routledge et al., 1991, Eur J Immunol, 21: 2717-2725), HUM291 (see, for example, Norman et al., 2000, Clinical Transplantation, 70(12): 1707-1712), teplizumab (see, for example, Herold et al., 2009, Clin Immunol, 132: 166-173), huCLB-T3/4 (see, for example, Labrijn et al., 2013, Proceedings of the National Academy of Sciences, 110(13): 5145-5150), otelixizumab (see, for example, Keymeulen et al., 2010, Diabetologia, 53: 614-623), blinatumomab (see, for example, Cheadle, 2006, Curr Opin Mol Ther, 8(1): 62-68), MT110 (see, for example, Silke and Gires, 2011, MAbs, 3(1): 31-37), catumaxomab (see, for example, Heiss and Murawa, 2010, Int J Cancer, 127(9): 2209-2221.

In certain embodiments, the scFv in an anti-CD117 bispecific binding polypeptide, or fragments thereof, binds to the same epitope as a CD3-specific antibody known in the art. In a specific embodiment, the scFv in a bispecific binding molecule of the invention binds to the same epitope as the CD3-specific antibody huOKT3. Binding to the same epitope can be determined by assays known to one skilled in the art, such as, for example, mutational analyses or crystallographic studies. In certain embodiments, the scFv competes for binding to CD3 with an antibody known in the art. In a specific embodiment, the scFv in a bispecific binding molecule of the invention competes for binding to CD3 with the CD3-specific antibody huOKT3. Competition for binding to CD3 can be determined by assays known to one skilled in the art, such as, for example, flow cytometry. In certain embodiments, the scFv comprises a VH with at least 85%, 90%, 95%, 98%, or at least 99% similarity to the VH of a CD3-specific antibody known in the art. In certain embodiments, the scFv comprises the VH of a CD3-specific antibody known in the art, comprising between 1 and 5 conservative amino acid substitutions. In certain embodiments, the scFv comprises a VL with at least 85%, 90%, 95%, 98%, or at least 99% similarity to the VL of a CD3-specific antibody known in the art. In certain embodiments, the scFv comprises the VL of a CD3-specific antibody known in the art, comprising between 1 and 5 conservative amino acid substitutions.

The anti-CD117 bispecific binding polypeptides, or fragments thereof, described herein may also include modifications and/or mutations that alter the properties of the antibodies and/or fragments, such as those that increase half-life, increase or decrease ADCC, etc., as is known in the art.

In one embodiment, the anti-CD117 bispecific binding polypeptides, or fragments thereof, comprises a variant Fc region, wherein said variant Fc region comprises at least one amino acid modification relative to a wild-type Fc region, such that said molecule has an altered affinity for an Fc gammaR. Certain amino acid positions within the Fc region are known through crystallography studies to make a direct contact with FcγR. Specifically, amino acids 234-239 (hinge region), amino acids 265-269 (B/C loop), amino acids 297-299 (C′/E loop), and amino acids 327-332 (F/G) loop. (see Sondermann et al., 2000 Nature, 406: 267-273). For example, amino acid substitutions at amino acid positions 234 and 235 of the Fc region have been identified as decreasing affinity of an IgG antibody for binding to an Fc receptor, particularly an Fc gamma receptor (FcγR). In one embodiment, an anti-CD117 antibody described herein comprises an Fc region comprising an amino acid substitution at L234 and/or L235, e.g., L234A and L235A (EU index). Thus, the anti-CD117 bispecific binding polypeptides, or fragments thereof described herein may comprise variant Fc regions comprising modification of at least one residue that makes a direct contact with an FcγR based on structural and crystallographic analysis. In one embodiment, the Fc region of the anti-CD117 bispecific binding polypeptides, or fragments thereof (or Fc containing fragment thereof) comprises an amino acid substitution at amino acid 265 according to the EU index as in Kabat et al., Sequences of Proteins of Immunological Interest, 5th Ed. Public Health Service, NH1, MD (1991), expressly incorporated herein by references. The “EU index as in Kabat” or “EU index” refers to the numbering of the human IgG1 EU antibody and is used herein in reference to Fc amino acid positions unless otherwise indicated.

In one embodiment, the Fc region comprises a D265A mutation. In one embodiment, the Fc region comprises a D265C mutation.

In some embodiments, the Fc region of the anti-CD117 bispecific binding polypeptides, or fragments thereof (or fragment thereof) comprises an amino acid substitution at amino acid 234 according to the EU index as in Kabat. In one embodiment, the Fc region comprises a L234A mutation. In some embodiments, the Fc region of the anti-CD117 bispecific binding polypeptides, or fragments thereof (or fragment thereof) comprises an amino acid substitution at amino acid 235 according to the EU index as in Kabat. In one embodiment, the Fc region comprises a L235A mutation. In yet another embodiment, the Fc region comprises a L234A and L235A mutation. In a further embodiment, the Fc region comprises a D265C, L234A, and L235A mutation.

In certain aspects a variant IgG Fc domain comprises one or more amino acid substitutions resulting in decreased or ablated binding affinity for an Fc gammaR and/or Clq as compared to the wild type Fc domain not comprising the one or more amino acid substitutions. Fc binding interactions are essential for a variety of effector functions and downstream signaling events including, but not limited to, antibody dependent cell-mediated cytotoxicity (ADCC) and complement dependent cytotoxicity (CDC). Accordingly, in certain aspects, a bispecific binding polypeptides, or fragments thereof comprising a modified Fc region (e.g., comprising a L234A, L235A, and a D265C mutation) has substantially reduced or abolished effector functions.

Affinity to an Fc region can be determined using a variety of techniques known in the art, for example but not limited to, equilibrium methods (e.g., enzyme-linked immunoabsorbent assay (ELISA); KinExA, Rathanaswami et al. Analytical Biochemistry, Vol. 373:52-60, 2008; or radioimmunoassay (RIA)), or by a surface plasmon resonance assay or other mechanism of kinetics-based assay (e.g., BIACORE™ analysis or Octet™ analysis (forteBIO)), and other methods such as indirect binding assays, competitive binding assays fluorescence resonance energy transfer (FRET), gel electrophoresis and chromatography (e.g., gel filtration). These and other methods may utilize a label on one or more of the components being examined and/or employ a variety of detection methods including but not limited to chromogenic, fluorescent, luminescent, or isotopic labels. A detailed description of binding affinities and kinetics can be found in Paul, W. E., ed., Fundamental Immunology, 4th Ed., Lippincott-Raven, Philadelphia (1999), which focuses on antibody-immunogen interactions. One example of a competitive binding assay is a radioimmunoassay comprising the incubation of labeled antigen with the antibody of interest in the presence of increasing amounts of unlabeled antigen, and the detection of the antibody bound to the labeled antigen. The affinity of the antibody of interest for a particular antigen and the binding off-rates can be determined from the data by scatchard plot analysis. Competition with a second antibody can also be determined using radioimmunoassays. In this case, the antigen is incubated with antibody of interest conjugated to a labeled compound in the presence of increasing amounts of an unlabeled second antibody.

In one embodiment, an anti-CD117 bispecific binding polypeptides, or fragments thereof described herein comprises an Fc region comprising L235A, L235A, and D265C (EU index). The bispecific binding polypeptides, or fragments thereof of the invention may be further engineered to further modulate antibody half-life by introducing additional Fc mutations, such as those described for example in (Dall'Acqua et al. (2006) J Biol Chem 281: 23514-24), (Zalevsky et al. (2010) Nat Biotechnol 28: 157-9), (Hinton et al. (2004) J Biol Chem 279: 6213-6), (Hinton et al. (2006) J Immunol 176: 346-56), (Shields et al. (2001) J Biol Chem 276: 6591-604), (Petkova et al. (2006) Int Immunol 18: 1759-69), (Datta-Mannan et al. (2007) Drug Metab Dispos 35: 86-94), (Vaccaro et al. (2005) Nat Biotechnol 23: 1283-8), (Yeung et al. (2010) Cancer Res 70: 3269-77) and (Kim et al. (1999) Eur J Immunol 29: 2819-25), and include positions 250, 252, 253, 254, 256, 257, 307, 376, 380, 428, 434 and 435. Exemplary mutations that may be made singularly or in combination are T250Q, M252Y, 1253A, S254T, T256E, P2571, T307A, D376V, E380A, M428L, H433K, N434S, N434A, N434H, N434F, H435A and H435R mutations.

Thus, in one embodiment, the Fc region comprises a mutation resulting in a decrease in half life. A bispecific binding polypeptides, or fragments thereof, having a short half life (also referred to herein as a “fast” half life) may be advantageous in certain instances where the bispecific binding polypeptides, or fragments thereof, is expected to function as a short-lived therapeutic, e.g., the conditioning step described herein where the bispecific binding polypeptides, or fragments thereof, is administered followed by HSCs. Ideally, the bispecific binding polypeptides, or fragments thereof would be substantially cleared prior to delivery of the HSCs, which also generally express CD117 but are not the target of the anti-CD117 bispecific binding polypeptides, or fragments thereof, unlike the endogenous stem cells. In one embodiment, the Fc region comprises a mutation at position 435 (EU index according to Kabat). In one embodiment, the mutation is an H435A mutation. In another embodiment, the mutation is a D265C mutation. In yet another embodiment, the mutations are an H435A mutation and a D265C mutation.

In one embodiment, the anti-CD117 bispecific binding polypeptides, or fragments thereof described herein have a half life of equal to or less than 24 hours, equal to or less than 22 hours, equal to or less than 20 hours, equal to or less than 18 hours, equal to or less than 16 hours, equal to or less than 14 hours, equal to or less than 13 hours, equal to or less than 12 hours, equal to or less than 11 hours, equal to or less than 10 hours, equal to or less than 9 hours, equal to or less than 8 hours, equal to or less than 7 hours, equal to or less than 6 hours, or equal to or less than 5 hours. In one embodiment, the half life of the antibody is 5 hours to 7 hours; is 5 hours to 9 hours; is 15 hours to 11 hours; is 5 hours to 13 hours; is 5 hours to 15 hours; is 5 hours to 20 hours; is 5 hours to 24 hours; is 7 hours to 24 hours; is 9 hours to 24 hours; is 11 hours to 24 hours; 12 hours to 22 hours; 10 hours to 20 hours; 8 hours to 18 hours; or 14 hours to 24 hours.

Anti-CD117 bispecific binding polypeptides, or fragments thereof, that can be used in conjunction with the patient conditioning methods described herein include, for instance, antibody portions produced and released from ATCC Accession No. 10716 (deposited as BA7.3C.9), such as the SR-1 antibody, which is described, for example, in U.S. Pat. No. 5,489,516, the disclosure of which is incorporated herein by reference as it pertains to anti-CD117 antibodies.

In one embodiment, an anti-CD117 bispecific binding polypeptides, or fragments thereof described herein comprises an Fc region comprising L235A, L235A, D265C, and H435A (EU index).

Additional anti-CD117 bispecific binding polypeptides, or fragments thereof that can be used in conjunction with the patient conditioning methods described herein include those described in U.S. Pat. No. 7,915,391, which describes, e.g., humanized SR-1 antibodies; U.S. Pat. No. 5,808,002, which describes, e.g., the anti-CD117 A3C6E2 antibody, as well as those described in, for example, WO 2015/050959, which describes anti-CD117 antibodies that bind epitopes containing Pro317, Asn320, Glu329, Val331, Asp332, Lus358, Glue360, Glue376, His378, and/or Thr380 of human CD117; and US 2012/0288506 (also published as U.S. Pat. No. 8,552,157), which describes, e.g., the anti-CD117 antibody CK6, having the CDR sequences of:

a CDR-H1 having the amino acid sequence
(SEQ ID NO: 1)
SYWIG;
a CDR-H2 having the amino acid sequence
(SEQ ID NO: 2)
IIYPGDSDTRYSPSFQG;
a CDR-H3 having the amino acid sequence
(SEQ ID NO: 3)
HGRGYNGYEGAFDI;
a CDR-L1 having the amino acid sequence
(SEQ ID NO: 4)
RASQGISSALA;
a CDR-L2 having the amino acid sequence
(SEQ ID NO: 5)
DASSLES;
and
a CDR-L3 having the amino acid sequence
(SEQ ID NO: 6)
CQQFNSYPLT

The heavy chain variable region amino acid sequence of CK6 is provided in SEQ ID NO: 27):

(SEQ ID NO: 29; CDRs are underlined are in bold)
QVQLVQSGAAVKKPGESLKISCKGSGYRFTSYWIGWVRQMPGKGL
EWMGIIYPGDSDTRYSPSFQGQVTI SAGKSISTAYLQWSSLKASD
TAMYYCARHGRGYNGYEGAFDIWGQGTMVTVSS.

The light chain amino acid variable sequence of CK6 is provided in SEQ ID NO: 28:

(SEQ ID NO: 30; CDRs are underlined and in bold)
AIQLTQSPSSLSASVGDRVTITCRASQGISSALAWYQQKPGKAPKLL
IYDASSLESGVPSRFSGSGSGTD FTLTISSLQPEDFATYYCQQFNS
YPLTFGGGTKVEIK.

Additional anti-CD117 bispecific binding polypeptides, or fragments thereof that may be used in conjunction with the compositions and methods described herein include those described in US 2015/0320880, such as the clones 9P3, NEG024, NEG027, NEG085, NEG086, and 20376.

The disclosures of each of the foregoing publications are incorporated herein by reference as they pertain to anti-CD117 antibodies. Anti-CD117 bispecific binding polypeptides, or fragments thereof that may be used in conjunction with the compositions and methods described herein include the above-described antibodies and antigen-binding fragments thereof, as well as humanized variants of those non-human antibodies and antigen-binding fragments described above and antibodies or antigen-binding fragments that bind the same epitope as those described above, as assessed, for instance, by way of a competitive CD117 binding assay.

Exemplary antigen-binding fragments of the foregoing antibodies include a dual-variable immunoglobulin domain, a single-chain Fv molecule (scFv), a diabody, a triabody, a nanobody, an antibody-like protein scaffold, a Fv fragment, a Fab fragment, a F(ab′)₂ molecule, and a tandem di-scFv, among others.

Anti-CD117 bispecific binding polypeptides, or fragments thereof may be produced using recombinant methods and compositions, e.g., as described in U.S. Pat. No. 4,816,567. In one embodiment, isolated nucleic acid encoding an anti-CD117 bispecific binding polypeptides, or fragments thereof described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., Y0, NS0, Sp20 cell). In one embodiment, a method of making an anti-CLL-1 antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an anti-CD117 bispecific binding polypeptides, or fragments thereof, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as YO, NSO and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

In one embodiment, the anti-CD117 bispecific binding polypeptides, or fragments thereof, comprises variable regions having an amino acid sequence that is at least 95%, 96%, 97% or 99% identical to the SEQ ID Nos disclosed herein. Alternatively, the anti-CD117 bispecific binding polypeptides, or fragments thereof, comprises CDRs comprising the SEQ ID Nos disclosed herein with framework regions of the variable regions described herein having an amino acid sequence that is at least 95%, 96%, 97% or 99% identical to the SEQ ID Nos disclosed herein.

In one embodiment, the anti-CD117 bispecific binding polypeptides, or fragments thereof, comprises a heavy chain variable region and a heavy chain constant region having an amino acid sequence that is disclosed herein. In another embodiment, the anti-CD117 bispecific binding polypeptides, or fragments thereof, comprises a light chain variable region and a light chain constant region having an amino acid sequence that is disclosed herein. In yet another embodiment, the anti-CD117 bispecific binding polypeptides, or fragments thereof, comprises a heavy chain variable region, a light chain variable region, a heavy chain constant region and a light chain constant region having an amino acid sequence that is disclosed herein.

### Methods of Preparing Bispecific Antibodies

Bispecific antibodies can be prepared according to standard methods known in the art, including, in some embodiments, as either full length antibodies or as antibody fragments (e.g. F(ab)2 bispecific antibodies, etc.). Traditional production of full length bispecific antibodies is based on the co-expression of two immunoglobulin heavy chain-light chain pairs, where the two chains have different specificities. Due to the random pairing of immunoglobulin heavy and light chains, a potential mixture of different antibody molecules may be produced, with generally one correct pairing of the bispecific heterodimer. Purification of the correct heterodimer molecule is usually done by affinity chromatography.

Bispecific antibodies may also be produced using heavy chain heterodimerization methods. Such methods include the “knob-in-hole” method, which is described in e.g., U.S. Pat. Nos. 7,695,936, 5,807,706 and U.S. Patent Application Publication 2003/0078385, which are hereby incorporated herein by reference in their entirety. In the “knob-in-hole” method, a “protrusion” is generated by replacing one or more small amino acid side chains (e.g. alanine or threonine) from the interface of a first antibody molecule with larger side chains (e.g. tyrosine or tryptophan). Compensatory “cavities” of identical or similar size to the large side chain(s) are created on the interface of a second antibody molecule by replacing amino acid having large side chains with amino acids having smaller ones (e.g. alanine or threonine). This provides a mechanism for increasing the yield of the heterodimer over other unwanted end-products such as homodimers. In many embodiments of the bispecific antibodies produced by the “knob-in-hole” method, an Fc region contains one pair of knobs and holes substitutions. In some embodiments, the Fc region of a first heavy chain, i.e., chain A, contains one or more amino acid substitutions, while the Fc region of a second heavy chain, i.e., chain B, contains one or more amino acid substitutions. For example, the following knobs and holes substitutions in chain A and chain B of an IgG1 Fc region have been found to increase heterodimer formation as compared with that found with unmodified chain A and chain B: 1) Y407T in chain A and T366Y in chain B; 2) Y407A in chain A and T366W in chain B; 3) F405A in chain A and T394W in chain B; 4) F405W in chain A and T394S in chain B; 5) Y407T in chain A and T366Y in chain B; 6) T366Y and F405A in chain A and T394W and Y407T in chain B; 7) T366W and F405W in chain A and T394S and Y407A in chain B; 8) F405W and Y407A in chain A and T366W and T394S in chain B; and 9) T366W in chain A and T366S, L368A, and Y407V in chain B. Similarly, substitutions changing the charge of one or more amino acid residue, for example, one or more amino acid residue in the CH3-CH3 interface, can enhance heterodimer formation as described in WO 2009/089004, which is hereby incorporated herein by reference in its entirety. Such substitutions are referred to herein as “charge pair substitutions,” and thus, an Fc region containing one or more charge pair substitutions in the A chain may contain different substitution(s) in the B chain. General examples of charge pair substitutions include the following: 1) K409D or K409E in chain A and D399K or D399R in chain B; 2) K392D or K392E in chain A and D399K or D399R in chain B; 3) K439D or K439E in chain A and E356K or E356R in chain B; and 4) K370D or K370E in chain A and E357K or E357R in chain B. In some embodiments, the bispecific antibodies, or portions thereof, may also be produced using a common light chain. Use of a common light chain can decrease the number of possible mispairings, as described in WO 98/50431, the contents of which are incorporated by reference in its entirety. One or more of the these “knob-in-hole” and/or “charge pair substitutions,” can be used in the Fc regions of the heterodimeric bispecific antibodies described herein.

In some embodiments, the method of producing a bispecific antibody using the “knob-in-hole” method includes incubating a first protein molecule comprising a heavy chain with the “knob” mutation together with a second protein molecule comprising a heavy chain with the “hole” mutation under reducing conditions sufficient to allow the cysteines in the hinge region to undergo disulfide-bond isomerization. Examples of suitable conditions are described in U.S. Patent Application Publication 2016/0046727, which is hereby incorporated herein by reference in its entirety. In some embodiments, the minimal requirements for the cysteines in the hinge region for undergoing disulfide-bond isomerization may differ depending on the homodimeric starting proteins, in particular depending on the exact sequence in the hinge region. In some embodiments, the respective homodimeric interactions of the first and the second heavy chain CH3 regions with the “knob” and “hole” mutations are sufficiently weak to allow cysteines in the hinge region to undergo disulfide-bond isomerization under the given conditions. In some embodiments, the reducing conditions include the addition of a reducing agent, e.g. a reducing agent selected from the group consisting of: tripeptide glutathione (GSH), 2-mercaptoethylamine (2-MEA), dithiothreitol (DTT), dithioerythritol (DTE), glutathione, tris(2-carboxyethyl)phosphine (TCEP), L-cysteine and beta-mercapto-ethanol. In other embodiments, the reducing conditions are described in terms of the required redox potential. The redox potential, which takes into consideration the stoichiometry of two GSH oxidized per GSSG is a quantitative measure for the redox state. In some embodiments the reaction is performed under reducing conditions with a redox potential ranging below −50 mV, such as below −150 mV, between −150 and −600 mV, such as between −200 and −500 mV, between −250 and −450 mV, such as between −250 and −400 mV, between −260 and −300 mV. In other embodiments described herein to produce bispecific antibody using the “knob-in-hole” method includes restoring the conditions to become non-reducing or less reducing after the completion of the ruction reaction, for example by removal of a reducing agent, e.g. by desalting.

The variant heavy chain molecules provided herein produced using the “knob-in-hole” method may be useful for the generation of bispecific antibodies and overcome the limitations and technical difficulties (e.g., improper pairing) noted above. In some embodiments, one heavy chain and one light chain within an antibody are modified whereby a native cysteine is substituted by a non-cysteine amino acid, and a native non-cysteine amino acid is substituted by a cysteine amino acid. Such modifications provided herein are generated in the heavy chain (HC) and light chain (LC) domains and result in the relocation of an HC-LC interchain disulfide bridge. In other embodiments, when generating a bispecific antibody from four separate polypeptides, for example, where the modified arm has a binding specificity for one target and the unmodified arm has a binding specificity for a different target, the four polypeptides will assemble such that the modified heavy chain polypeptide properly hybridizes with the modified light chain and the unmodified heavy chain properly hybridizes with the unmodified light chain. As used herein, the term “unmodified” refers to heavy and light chains that do not contain the HC-LC modifications (e.g., “knob-in-hole” or the cysteine modifications) as described herein in the CH2 and/or CH3 regions described herein and/or known in the art. In some embodiments, the HC-LC modifications provided herein can be combined with further modifications of the heavy chain, particularly in the CH2 and/or CH3 regions to ensure proper heavy chain heterodimerization and/or to enhance purification of the heavy chain heterodimer.

### Methods of Identifying Bispecific Binding Proteins

Methods for high throughput screening of bispecific binding proteins, or fragments thereof, libraries for molecules capable of binding CD117 (e.g., GNNK+ CD117) (and/or CD3) can be used to identify and affinity mature antibodies useful for treating cancers, autoimmune diseases, and conditioning a patient (e.g., a human patient) in need of hematopoietic stem cell therapy as described herein. Such methods include in vitro display techniques known in the art, such as phage display, bacterial display, yeast display, mammalian cell display, ribosome display, mRNA display, and cDNA display, among others. The use of phage display to isolate ligands that bind biologically relevant molecules has been reviewed, for example, in Felici et al., Biotechnol. Annual Rev. 1:149-183, 1995; Katz, Annual Rev. Biophys. Biomol. Struct. 26:27-45, 1997; and Hoogenboom et al., Immunotechnology 4:1-20, 1998, the disclosures of each of which are incorporated herein by reference as they pertain to in vitro display techniques. Randomized combinatorial peptide libraries have been constructed to select for polypeptides that bind cell surface antigens as described in Kay, Perspect. Drug Discovery Des. 2:251-268, 1995 and Kay et al., Mol. Divers. 1:139-140, 1996, the disclosures of each of which are incorporated herein by reference as they pertain to the discovery of antigen-binding molecules. Proteins, such as multimeric proteins, have been successfully phage-displayed as functional molecules (see, for example, EP 0349578; EP 4527839; and EP 0589877, as well as Chiswell and McCafferty, Trends Biotechnol. 10:80-84 1992, the disclosures of each of which are incorporated herein by reference as they pertain to the use of in vitro display techniques for the discovery of antigen-binding molecules). In addition, functional antibody fragments, such as Fab and scFv fragments, have been expressed in in vitro display formats (see, for example, McCafferty et al., Nature 348:552-554, 1990; Barbas et al., Proc. Natl. Acad. Sci. USA 88:7978-7982, 1991; and Clackson et al., Nature 352:624-628, 1991, the disclosures of each of which are incorporated herein by reference as they pertain to in vitro display platforms for the discovery of antigen-binding molecules). These techniques, among others, can be used to identify and improve the affinity of antibodies that bind CD117 (e.g., GNNK+ CD117) (and/or CD3) that can in turn be used to deplete endogenous hematopoietic stem cells in a patient (e.g., a human patient) in need of hematopoietic stem cell transplant therapy.

In addition to in vitro display techniques, computational modeling techniques can be used to design and identify bispecific binding proteins, or fragments thereof, in silico that bind CD117 (e.g., GNNK+ CD117) (and/or CD3). For example, using computational modeling techniques, one of skill in the art can screen libraries of bispecific binding proteins, antibodies, or antibody fragments, in silico for molecules capable of binding specific epitopes, such as extracellular epitopes of this antigen. The bispecific binding proteins, antibodies, or antigen-binding fragments thereof, identified by these computational techniques can be used in conjunction with the therapeutic methods described herein, such as the cancer and autoimmune disease treatment methods described herein and the patient conditioning procedures described herein.

Additional techniques can be used to identify bispecific binding proteins, antibodies, or antigen-binding fragments thereof, that bind CD117 (e.g., GNNK+ CD117) (and/or CD3) on the surface of a cell (e.g., a cancer cell, autoimmune cell, or hematopoietic stem cell) and that are internalized by the cell, for instance, by receptor-mediated endocytosis. For example, the in vitro display techniques described above can be adapted to screen for bispecific binding proteins, antibodies, or antigen-binding fragments thereof, that bind CD117 (e.g., GNNK+ CD117) (and/or CD3) on the surface of a cancer cell, autoimmune cell, or hematopoietic stem cell and that are subsequently internalized. Phage display represents one such technique that can be used in conjunction with this screening paradigm. To identify bispecific binding proteins, antibodies, or fragments thereof, that bind CD117 (e.g., GNNK+ CD117) (and/or CD3) and are subsequently internalized by cancer cells, autoimmune cells, or hematopoietic stem cells, one of skill in the art can adapt the phage display techniques described, for example, in Williams et al., Leukemia 19:1432-1438, 2005, the disclosure of which is incorporated herein by reference in its entirety. For example, using mutagenesis methods known in the art, recombinant phage libraries can be produced that encode bispecific binding proteins, antibodies, antibody fragments, such as scFv fragments, Fab fragments, diabodies, triabodies, and ¹⁰Fn3 domains, among others, that contain randomized amino acid cassettes (e.g., in one or more, or all, of the CDRs or equivalent regions thereof or a bispecific binding proteins, antibody or antibody fragment). The framework regions, hinge, Fc domain, and other regions of the bispecific binding proteins, antibodies or antibody fragments may be designed such that they are non-immunogenic in humans, for instance, by virtue of having human germline antibody sequences or sequences that exhibit only minor variations relative to human germline antibodies.

Using phage display techniques described herein or known in the art, phage libraries containing randomized bispecific binding proteins, antibodies, or antibody fragments, covalently bound to the phage particles can be incubated with CD117 (e.g., GNNK+ CD117) (and/or CD3) antigen, for instance, by first incubating the phage library with blocking agents (such as, for instance, milk protein, bovine serum albumin, and/or IgG so as to remove phage encoding bispecific binding proteins, antibodies, or fragments thereof, that exhibit non-specific protein binding and phage that encode bispecific binding proteins, antibodies or fragments thereof that bind Fc domains, and then incubating the phage library with a population of hematopoietic stem cells. The phage library can be incubated with the target cells, such as cancer cells, autoimmune cells, or hematopoietic stem cells for a time sufficient to allow anti-CD117-specific bispecific binding proteins, antibodies, or antigen-binding fragments thereof, (e.g., GNNK+ CD117-specific antibodies, or antigen-binding fragments thereof) to bind cell-surface CD117 (e.g., sell-surface GNNK+ CD117) (and/or CD3) antigen and to subsequently be internalized by the cancer cells, autoimmune cells, or hematopoietic stem cells (e.g., from 30 minutes to 6 hours at 4° C., such as 1 hour at 4° C.). Phage containing bispecific binding proteins, antibodies, or fragments thereof, that do not exhibit sufficient affinity for one or more of these antigens so as to permit binding to, and internalization by, cancer cells, autoimmune cells, or hematopoietic stem cells can subsequently be removed by washing the cells, for instance, with cold (4° C.) 0.1 M glycine buffer at pH 2.8. Phage bound to bispecific binding proteins, antibodies, or fragments thereof, or that have been internalized by the cancer cells, autoimmune cells, or hematopoietic stem cells can be identified, for instance, by lysing the cells and recovering internalized phage from the cell culture medium. The phage can then be amplified in bacterial cells, for example, by incubating bacterial cells with recovered phage in 2×YT medium using methods known in the art. Phage recovered from this medium can then be characterized, for instance, by determining the nucleic acid sequence of the gene(s) encoding the bispecific binding proteins, antibodies, or fragments thereof, inserted within the phage genome. The encoded bispecific binding proteins, antibodies, or fragments thereof, or can subsequently be prepared de novo by chemical synthesis (for instance, of antibody fragments, such as scFv fragments) or by recombinant expression (for instance, of full-length antibodies).

The internalizing capacity of the prepared bispecific binding proteins, antibodies, or fragments thereof, can be assessed, for instance, using radionuclide internalization assays known in the art. For example, bispecific binding proteins, antibodies, or fragments thereof, identified using in vitro display techniques described herein or known in the art can be functionalized by incorporation of a radioactive isotope, such as ¹⁸F, ⁷⁵Br, ⁷⁷Br, ¹²²I, ¹²³I, ¹²⁴I, ¹²⁵I, ¹²⁹I, ¹³¹I ²¹¹At, ⁶⁷Ga, ¹¹¹In, ⁹⁹Tc, ¹⁶⁹Yb, ¹⁸⁶Re, ⁶⁴Cu, ⁶⁷Cu, ¹⁷⁷Lu, ⁷⁷As, ⁷²As, ⁸⁶Y, ⁹⁰Y, ⁸⁹Zr, ²¹²Bi, ²¹³Bi, or ²²⁵Ac. For instance, radioactive halogens, such as ¹⁸F, ⁷⁵Br, ⁷⁷Br, ¹²²I, ¹²³I, ¹²⁴I, ¹²⁵I, ¹²⁹I, ¹³¹I, ²¹¹At, can be incorporated into bispecific binding proteins, antibodies, or fragments thereof, using beads, such as polystyrene beads, containing electrophilic halogen reagents (e.g., lodination Beads, Thermo Fisher Scientific, Inc., Cambridge, Mass.). Radiolabeled bispecific binding proteins, antibodies, or fragments thereof, can be incubated with cancer cells, autoimmune cells, or hematopoietic stem cells for a time sufficient to permit internalization (e.g., from 30 minutes to 6 hours at 4° C., such as 1 hour at 4° C.). The cells can then be washed to remove non-internalized antibodies, or fragments thereof, (e.g., using cold (4° C.) 0.1 M glycine buffer at pH 2.8). Internalized bispecific binding proteins, antibodies, or fragments thereof, can be identified by detecting the emitted radiation (e.g., γ-radiation) of the resulting cancer cells, autoimmune cells, or hematopoietic stem cells in comparison with the emitted radiation (e.g., γ-radiation) of the recovered wash buffer.

### Methods of Treatment and Targeted Cell Depletion

As described herein, hematopoietic stem cell transplant therapy can be administered to a subject in need of treatment so as to populate or re-populate one or more blood cell types. Hematopoietic stem cells generally exhibit multi-potency, and can thus differentiate into multiple different blood lineages including, but not limited to, granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). Hematopoietic stem cells are additionally capable of self-renewal, and can thus give rise to daughter cells that have equivalent potential as the mother cell, and also feature the capacity to be reintroduced into a transplant recipient whereupon they home to the hematopoietic stem cell niche and re-establish productive and sustained hematopoiesis.

Hematopoietic stem cells can thus be administered to a patient defective or deficient in one or more cell types of the hematopoietic lineage in order to re-constitute the defective or deficient population of cells in vivo, thereby treating the pathology associated with the defect or depletion in the endogenous blood cell population. The compositions and methods described herein can thus be used to treat a non-malignant hemoglobinopathy (e.g., a hemoglobinopathy selected from the group consisting of sickle cell anemia, thalassemia, Fanconi anemia, aplastic anemia, and Wiskott-Aldrich syndrome). Additionally or alternatively, the compositions and methods described herein can be used to treat an immunodeficiency, such as a congenital immunodeficiency. Additionally or alternatively, the compositions and methods described herein can be used to treat an acquired immunodeficiency (e.g., an acquired immunodeficiency selected from the group consisting of HIV and AIDS). The compositions and methods described herein can be used to treat a metabolic disorder (e.g., a metabolic disorder selected from the group consisting of glycogen storage diseases, mucopolysaccharidoses, Gaucher's Disease, Hurlers Disease, sphingolipidoses, and metachromatic leukodystrophy).

Additionally or alternatively, the compositions and methods described herein can be used to treat a malignancy or proliferative disorder, such as a hematologic cancer, myeloproliferative disease. In the case of cancer treatment, the compositions and methods described herein may be administered to a patient so as to deplete a population of endogenous hematopoietic stem cells prior to hematopoietic stem cell transplantation therapy, in which case the transplanted cells can home to a niche created by the endogenous cell depletion step and establish productive hematopoiesis. This, in turn, can re-constitute a population of cells depleted during cancer cell eradication, such as during systemic chemotherapy. Exemplary hematological cancers that can be treated using the compositions and methods described herein include, without limitation, acute myeloid leukemia, acute lymphoid leukemia, chronic myeloid leukemia, chronic lymphoid leukemia, multiple myeloma, diffuse large B-cell lymphoma, and non-Hodgkin's lymphoma, as well as other cancerous conditions, including neuroblastoma.

Additional diseases that can be treated with the compositions and methods described herein include, without limitation, adenosine deaminase deficiency and severe combined immunodeficiency, hyper immunoglobulin M syndrome, Chediak-Higashi disease, hereditary lymphohistiocytosis, osteopetrosis, osteogenesis imperfecta, storage diseases, thalassemia major, systemic sclerosis, systemic lupus erythematosus, multiple sclerosis, and juvenile rheumatoid arthritis.

The anti-CD3/anti-HC bispecific binding proteins, antibodies, antigen-binding fragments thereof, and conjugates described herein may be used to induce solid organ transplant tolerance. For instance, the compositions and methods described herein may be used to deplete or ablate a population of cells from a target tissue (e.g., to deplete hematopoietic stem cells from the bone marrow stem cell niche). Following such depletion of cells from the target tissues, a population of stem or progenitor cells from an organ donor (e.g., hematopoietic stem cells from the organ donor) may be administered to the transplant recipient, and following the engraftment of such stem or progenitor cells, a temporary or stable mixed chimerism may be achieved, thereby enabling long-term transplant organ tolerance without the need for further immunosuppressive agents. For example, the compositions and methods described herein may be used to induce transplant tolerance in a solid organ transplant recipient (e.g., a kidney transplant, lung transplant, liver transplant, and heart transplant, among others). The compositions and methods described herein are well-suited for use in connection the induction of solid organ transplant tolerance, for instance, because a low percentage temporary or stable donor engraftment is sufficient to induce long-term tolerance of the transplanted organ.

In addition, the compositions and methods described herein can be used to treat cancers directly, such as cancers characterized by cells that are CD117+. For instance, the compositions and methods described herein can be used to treat leukemia, particularly in patients that exhibit CD117+ leukemic cells. By depleting CD117+ cancerous cells, such as leukemic cells, the compositions and methods described herein can be used to treat various cancers directly. Exemplary cancers that may be treated in this fashion include hematological cancers, such as acute myeloid leukemia, acute lymphoid leukemia, chronic myeloid leukemia, chronic lymphoid leukemia, multiple myeloma, diffuse large B-cell lymphoma, and non-Hodgkin's lymphoma.

Acute myeloid leukemia (AML) is a cancer of the myeloid line of blood cells, characterized by the rapid growth of abnormal white blood cells that build up in the bone marrow and interfere with the production of normal blood cells. AML is the most common acute leukemia affecting adults, and its incidence increases with age. The symptoms of AML are caused by replacement of normal bone marrow with leukemic cells, which causes a drop in red blood cells, platelets, and normal white blood cells. As an acute leukemia, AML progresses rapidly and may be fatal within weeks or months if left untreated. In one embodiment, the anti-CD117 bispecific binding proteins described herein are used to treat AML in a human patient in need thereof. In certain embodiments the anti-CD117 bispecific binding proteins treatment depletes AML cells in the treated subjects. In some embodiments 50% or more of the AML cells are depleted. In other embodiments, 60% or more of the AML cells are depleted, or 70% or more of the AML cells are depleted, or 80% of more or 90% or more, or 95% or more of the AML cells are depleted. In certain embodiments the anti-CD117 bispecific binding proteins treatments are a single dose treatment. In certain embodiments the single dose anti-CD117 bispecific binding proteins treatment depletes 60%, 70%, 80%, 90% or 95% or more of the AML cells.

In addition, the compositions and methods described herein can be used to treat autoimmune disorders. For instance, an anti-CD3/CD117 bispecific antibody, or antigen-binding fragment thereof, can be administered to a subject, such as a human patient suffering from an autoimmune disorder, so as to kill a CD117+ immune cell. The CD117+ immune cell may be an autoreactive lymphocyte, such as a T-cell that expresses a T-cell receptor that specifically binds, and mounts an immune response against, a self antigen. By depleting self-reactive, CD117+ cells, the compositions and methods described herein can be used to treat autoimmune pathologies, such as those described below. Additionally or alternatively, the compositions and methods described herein can be used to treat an autoimmune disease by depleting a population of endogenous hematopoietic stem cells prior to hematopoietic stem cell transplantation therapy, in which case the transplanted cells can home to a niche created by the endogenous cell depletion step and establish productive hematopoiesis. This, in turn, can re-constitute a population of cells depleted during autoimmune cell eradication.

Autoimmune diseases that can be treated using the compositions and methods described herein include, without limitation, psoriasis, psoriatic arthritis, Type 1 diabetes mellitus (Type 1 diabetes), rheumatoid arthritis (RA), human systemic lupus (SLE), multiple sclerosis (MS), inflammatory bowel disease (IBD), lymphocytic colitis, acute disseminated encephalomyelitis (ADEM), Addison's disease, alopecia universalis, ankylosing spondylitisis, antiphospholipid antibody syndrome (APS), aplastic anemia, autoimmune hemolytic anemia, autoimmune hepatitis, autoimmune inner ear disease (AIED), autoimmune lymphoproliferative syndrome (ALPS), autoimmune oophoritis, Balo disease, Behcet's disease, bullous pemphigoid, cardiomyopathy, Chagas' disease, chronic fatigue immune dysfunction syndrome (CFIDS), chronic inflammatory demyelinating polyneuropathy, Crohn's disease, cicatrical pemphigoid, coeliac sprue-dermatitis herpetiformis, cold agglutinin disease, CREST syndrome, Degos disease, discoid lupus, dysautonomia, endometriosis, essential mixed cryoglobulinemia, fibromyalgia-fibromyositis, Goodpasture's syndrome, Grave's disease, Guillain-Barre syndrome (GBS), Hashimoto's thyroiditis, Hidradenitis suppurativa, idiopathic and/or acute thrombocytopenic purpura, idiopathic pulmonary fibrosis, IgA neuropathy, interstitial cystitis, juvenile arthritis, Kawasaki's disease, lichen planus, Lyme disease, Meniere disease, mixed connective tissue disease (MCTD), myasthenia gravis, neuromyotonia, opsoclonus myoclonus syndrome (OMS), optic neuritis, Ord's thyroiditis, pemphigus vulgaris, pernicious anemia, polychondritis, polymyositis and dermatomyositis, primary biliary cirrhosis, polyarteritis nodosa, polyglandular syndromes, polymyalgia rheumatica, primary agammaglobulinemia, Raynaud phenomenon, Reiter's syndrome, rheumatic fever, sarcoidosis, scleroderma, Sjögren's syndrome, stiff person syndrome, Takayasu's arteritis, temporal arteritis (also known as “giant cell arteritis”), ulcerative colitis, collagenous colitis, uveitis, vasculitis, vitiligo, vulvodynia (“vulvar vestibulitis”), and Wegener's granulomatosis.

The anti-CD117/CD3 bispecific binding proteins, antibodies, or antigen-binding fragments thereof, or described herein can be administered to a patient (e.g., a human patient suffering from cancer, an autoimmune disease, or in need of hematopoietic stem cell transplant therapy) in a variety of dosage forms. For instance, bispecific antibody binding proteins, or antigen-binding fragments thereof, described herein can be administered to a patient suffering from cancer, an autoimmune disease, or in need of hematopoietic stem cell transplant therapy in the form of an aqueous solution, such as an aqueous solution containing one or more pharmaceutically acceptable excipients. Pharmaceutically acceptable excipients for use with the compositions and methods described herein include viscosity-modifying agents. The aqueous solution may be sterilized using techniques known in the art.

Pharmaceutical formulations comprising the anti-CD117/CD3 bispecific binding proteins as described herein are prepared by mixing anti-CD117/CD3 bispecific binding proteins with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

The anti-CD117/CD3 bispecific binding proteins or antigen-binding fragments, described herein may be administered by a variety of routes, such as orally, transdermally, subcutaneously, intranasally, intravenously, intramuscularly, intraocularly, or parenterally. The most suitable route for administration in any given case will depend on the particular anti-CD117 bispecific binding proteins, or antigen-binding fragment, administered, the patient, pharmaceutical formulation methods, administration methods (e.g., administration time and administration route), the patient's age, body weight, sex, severity of the diseases being treated, the patient's diet, and the patient's excretion rate.

The effective dose of an anti-CD117/CD3 bispecific binding proteins, or antigen-binding fragment thereof, described herein can range, for example from about 0.001 to about 100 mg/kg of body weight per single (e.g., bolus) administration, multiple administrations, or continuous administration, or to achieve an optimal serum concentration (e.g., a serum concentration of 0.0001-5000 pg/mL) of the antibody, antigen-binding fragment thereof.

In one embodiment, the dose of the anti-CD117/CD3 bispecific binding proteins administered to the human patient is about 0.1 mg/kg to about 0.3 mg/kg.

In one embodiment, the dose of the anti-CD117/CD3 bispecific binding proteins administered to the human patient is about 0.15 mg/kg to about 0.3 mg/kg.

In one embodiment, the dose of the anti-CD117/CD3 bispecific binding proteins administered to the human patient is about 0.15 mg/kg to about 0.25 mg/kg.

In one embodiment, the dose of the anti-CD117/CD3 bispecific binding proteins administered to the human patient is about 0.2 mg/kg to about 0.3 mg/kg.

In one embodiment, the dose of the anti-CD117/CD3 bispecific binding proteins administered to the human patient is about 0.25 mg/kg to about 0.3 mg/kg.

In one embodiment, the dose of the anti-CD117/CD3 bispecific binding proteins administered to the human patient is about 0.1 mg/kg.

In one embodiment, the dose of the anti-CD117/CD3 bispecific binding proteins administered to the human patient is about 0.2 mg/kg.

In one embodiment, the dose of the anti-CD117/CD3 bispecific binding proteins administered to the human patient is about 0.3 mg/kg.

The dose may be administered one or more times (e.g., about 2-10 times) per day, week, or month to a subject (e.g., a human) suffering from cancer, an autoimmune disease, or undergoing conditioning therapy in preparation for receipt of a hematopoietic stem cell transplant. In the case of a conditioning procedure prior to hematopoietic stem cell transplantation, anti-CD117 bispecific binding proteins, or antigen-binding fragment thereof, can be administered to the patient at a time that optimally promotes engraftment of the exogenous hematopoietic stem cells, for instance, from about 1 hour to 1 week (e.g., 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 2 days, 3 days, 4 days, 5 days, 6 days, or 7 days) or more prior to administration of the exogenous hematopoietic stem cell transplant.

### EXAMPLES

The following examples are put forth so as to provide those of ordinary skill in the art with a description of how the compositions and methods described herein may be used, made, and evaluated, and are intended to be purely exemplary of the invention and are not intended to limit the scope of what the inventors regard as their invention. The CD3/CD117 bispecific antibodies bs-Ab-1, bs-Ab-2, and bs-Ab-3 are represented by the bispecific antibody described in FIG. 5. In addition to the various amino acid substitutions described for the bispecific antibodies in the following Examples, each bispecific antibody also comprises a LALA mutation in its Fc regions. Amino acid sequences of the binding regions of these bispecific antibodies are described in Table 4.

### Example 1. Production of the CD117/CD3 Bispecific Antibody Bs-Ab-1

Bispecific antibody bs-Ab-1, having a CD117-antigen binding arm and a CD3-antigen binding arm was prepared using the “knob-in-hole” heterodimerization technology using antigen binding regions from anti-CD117 antibody Ab85 and antigen binding regions from an anti-CD3 antibody Ab2 as described below.

bs-Ab-1 comprises the anti-CD117 heavy chain variable region sequence as set forth in SEQ ID NO: 13 and the light chain variable region sequence set forth in SEQ ID NO: 14, and was engineered to introduce the following Fc substitution, T366W. Using site-directed mutagenesis, the Fc region of the heavy chain sequence of anti-CD117 antibody Ab85 (i.e., SEQ ID NO: 150 as described in US 2019/0153114 A1, the content of which is hereby expressly incorporated by reference in its entirety) was engineered to introduce the following substitution, T366W (according to the EU index). The anti-CD117 Ab85 antibody variant, i.e., “Ab85 T366W,” was subsequently expressed.

The CD3-binding arm of bs-Ab-1 comprises the heavy chain variable region sequence as set forth in SEQ ID NO: 41 and the light chain variable region sequence as set forth in SEQ ID NO: 45. The anti-CD3 heavy chain and was engineered to introduce the following Fc substitutions, T366S, L368A and Y407V. The Fc region of the Ab2 (anti-CD3) antibody heavy chain sequence set forth SEQ ID NO: 49 was engineered to introduce the following substitutions, T366S, L368A and Y407V (amino acid positions refer to the Fc region according to the EU index). The Ab2 (anti-CD3) antibody variant, “anti-CD3 T366S L368A Y407V,” was subsequently expressed.

The Ab85 T366W parental antibody and anti-CD3 T366S L368A Y407V parental antibody were then assembled using standard knob-in-hole techniques to produce the bispecific heterodimer, bs-Ab-1.

The stability of bs-Ab-1 was evaluated after two rounds of freeze-thaw cycles, followed by analysis using size exclusion chromatography (SEC). To assess the level of aggregation, 20 pg of bs-Ab-1 was injected into a AdvanceBio SEC 300A column (Agilent Technologies). The eluted protein was detected using UV absorbance at 280 nm and and displayed no observable aggregation following two rounds of freeze-thaw cycles (data not shown). Further tests were performed using non-reduced capillary SDS PAGE (non-reduced CE-SDS). which determined that the level of partially reduced bispecific antibodies, was minimal, (the predominant species (>95%) in the population was bs-Ab-1).

bs-Ab-1 was also evaluated for its ability to bind to human CD117 (hCD117) using bio-layer interferometry on an OCTET® platform to confirm that the bispecific format did not interfere with the anti-CD117 arm of the bispecific antibody. Methods for determining binding were in accordance with those known in the art, for instance as set forth in Tobias et al., “Biomolecular Binding Kinetics Assays on the Octet Platform,” Application Note 14, 22 pages (2013). The binding assays were performed at 25° C. in phosphate buffered saline (0.1% BSA) using Bio-Layer Interferometry Device (ForteBio). Bs-Ab-1 was loaded on an OCTET® Anti-human IgG Fc Capture (AHC) biosensor, at a concentration of 66.7 nM. Then associated with 33 nM hCD117 antigen and dissociated with 33 nM hCD3 antigen, allowing bs-Ab-1 to bind to both antigens (CD3 and CD117). The binding response of bs-Ab 1 to hCD117 was confirmed, and the binding to hCD3 antigen was not detectable using this method (data not shown). Further, a baculovirus particle assay demonstrated that bs-Ab-1 did not exhibit non-specific binding. These results demonstrated the ability of bs-Ab-1 to bind hCD117 was maintained.

Finally, the thermostability of bs-Ab-1 was evaluated using differential scanning fluorimetry (DSF). 2 micrograms of bs-Ab-1 was combined with a protein thermal shift buffer and dye according to the protein thermal shift kit specifications (Applied Biosystems, Protein Thermal Shift Dye kit (Part #4461146) and was analyzed using Applied Biosystems Quant Studio 7 Flex instrument by Life Technologies and the melting temperature (Tm) of each antibody was determined. The data indicated that the bs-Ab-1 bispecific antibody showed high intrinsic thermal stability.

### Example 2. Analysis of an In Vitro Cell Killing Assay Using an Anti-CD117/Anti-CD3 Bispecific Antibody Bs-1-Ab

CD117 expressing target cells (Kasumi-1 cells) were cultured in the presence of the bs-Ab-1 from Example 1 for six days after which the number of viable CD117 expressing target cells were determined.

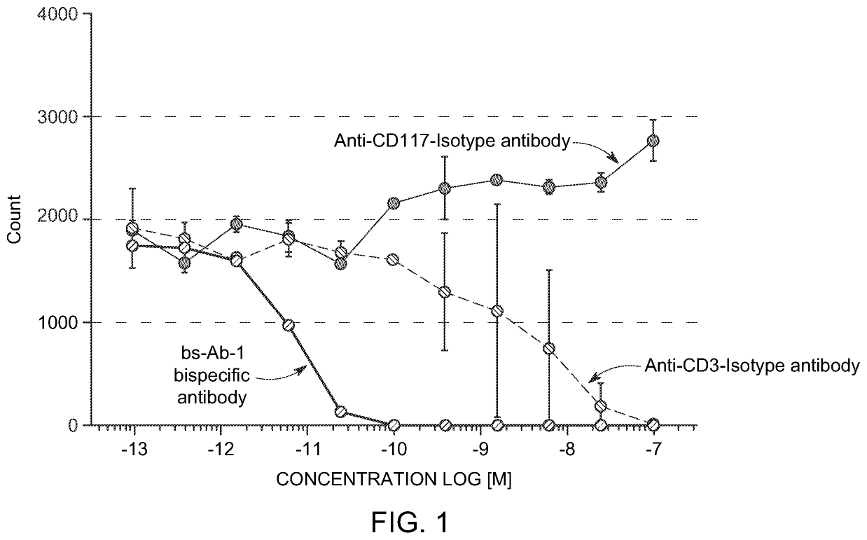

The results in FIG. 1 indicate that at day 6 the bs-Ab-1 was highly effective at killing CD117 expressing target cells in vitro, demonstrating significant killing of the CD117 expressing target cells (FIG. 1; IC₅₀=6.0 pM) in comparison to an anti-CD117-Isotype antibody (i.e., an antibody having one CD117 binding arm and one non-targeted binding (isotype) arm) and an anti-CD3-Isotype antibody (i.e., an antibody having one CD3 binding arm and one non-targeted binding (isotype) arm) (see FIG. 1; no significant depletion of the CD117 expressing target cells was observed). The IC₅₀ (pM) values and efficacy data for bs-Ab-1 from Example 1 are set forth in Table 1 below.

TABLE 1

| Day | IC₅₀ (pM) | Efficacy (%) |
|---|---|---|
| 3 | 11.7 | 84.0 |
| 4 | 6.4 | 97.6 |
| 5 | 7.6 | 98.3 |
| 6 | 6.0 | 99.9 |
| 7 | 6.2 | 99.7 |

Thus, bs-Ab-1 was highly effective at killing CD117 expressing target cells.

### Example 3. Analysis of an In Vitro Cell Killing Assay Using Bs-Ab-1

For in vitro cell killing assays using human hematopoietic stem cells, human bone marrow-derived CD34+ cells were cultured for seven days in the presence of either bs-Ab-1 from Example 1, an anti-CD3-Isotype antibody (i.e., an antibody having one CD3 binding arm and one non-binding (isotype) arm), or an anti-CD117-Isotype antibody (i.e., an antibody having one CD117 binding arm and one non-binding (isotype) arm). Cell viability was measured using flow cytometry.

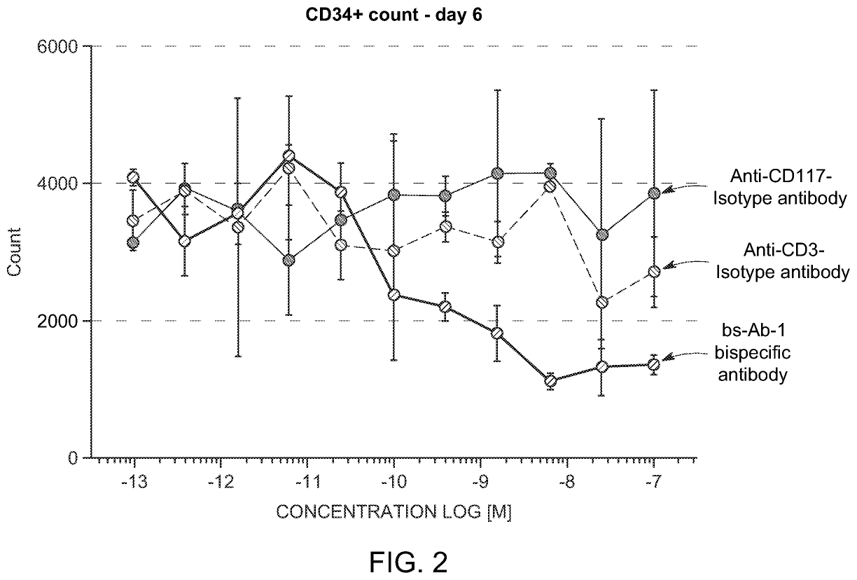

The results in FIG. 2 indicate that at day 6, bs-Ab-1 from Example 1 was effective at killing primary human CD34+ bone marrow cells in vitro (FIG. 2; IC₅₀=15.1 pM) in comparison to the anti-CD117-Isotype antibody and anti-CD3-Isotype antibody (FIG. 2; no significant depletion of the CD117 expressing target cells was observed).

The IC₅₀ (pM) values and efficacy data for bs-Ab-1 from Example 1 are set forth in Table 2 below.

TABLE 2

| Day | IC₅₀ (pM) | Efficacy (%) |
|---|---|---|
| 3 | 8737 | 15.3 |
| 4 | 233 | 39.3 |
| 5 | 35.3 | 49.7 |
| 6 | 15.1 | 64.8 |
| 7 | 23.8 | 70.9 |

Thus, bs-Ab-1 from Example 1 was effective at killing CD117 expressing cell lines (see Example 2) and primary human CD34+ cells (this Example).

### Example 4. In Vivo depletion assay using bs-Ab-1

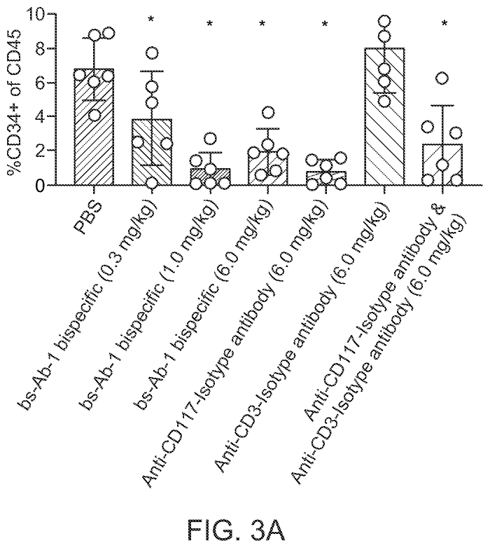

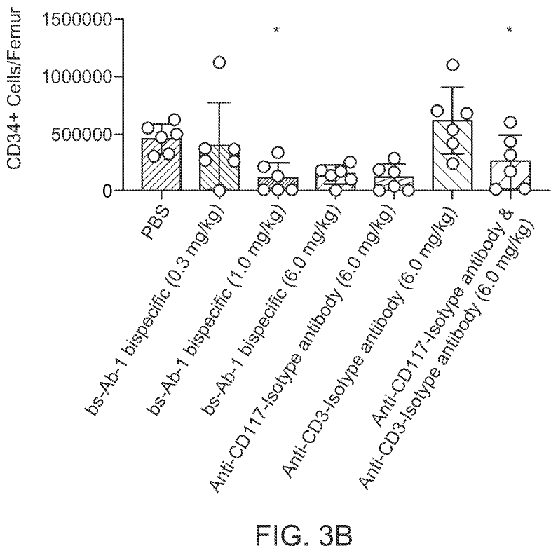

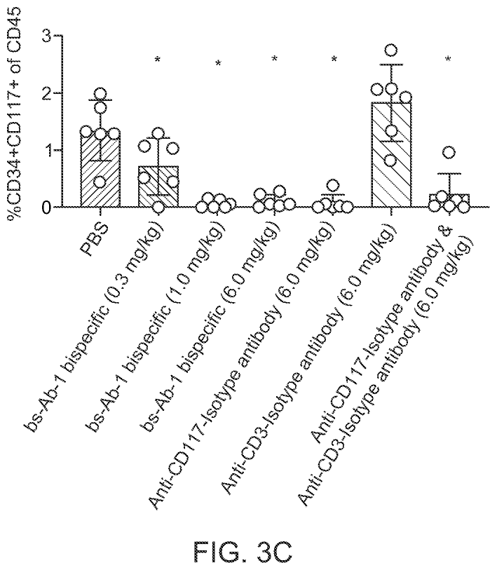

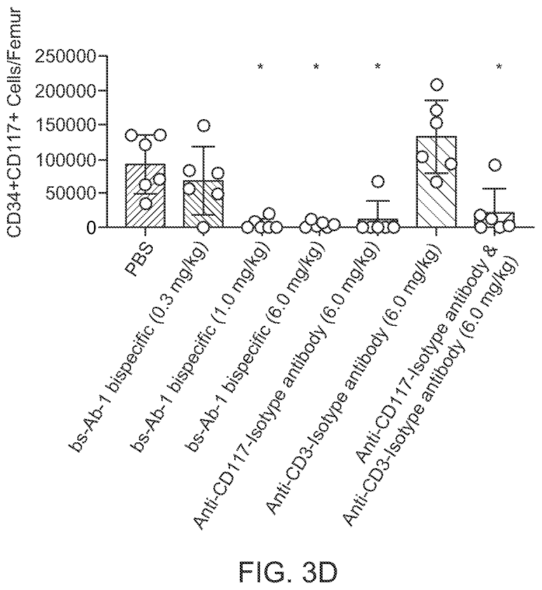

An in vivo depletion assay was performed to compare the ability of bs-Ab-1 from Example 1 to deplete cells compared to an anti-CD3-Isotype antibody, an anti-CD117-Isotype antibody and various controls (e.g., PBS (negative control)). The in vivo HSC depletion assay was conducted using humanized NSG mice (purchased from Jackson Laboratories). The bs-Ab-1 bispecific antibody from Example 1 was administered as a single injection of 0.3 mg/kg bs-Ab-1 bispecific antibody, 1.0 mg/kg bs-Ab-1 bispecific antibody, or 6.0 mg/kg bs-Ab-1 bispecific antibody to the humanized mouse model. In addition, an anti-CD117-isotype antibody (i.e., having one CD117 binding arm and one non-targeting arm), an anti-CD3-isotype antibody (i.e., having one CD3 binding arm and one non-targeting arm), and a combination of both an anti-CD117-isotype antibody and an anti-CD3-isotype antibody were similarly administered as a single injection of 6 mg/kg to the humanized mice on day 0. Bone marrow was collected on day 21 and examined by flow cytometry. The frequency (% cells maintained) and absolute number of CD34+ cells (FIGS. 3A and B) and CD34+CD117+ cells (FIGS. 3C and D), in the treated or control mice 21 days after a single administration are shown in FIGS. 3A-D.

The results indicate that humanized NSG mice treated with the bs-Ab-1 bispecific antibody from Example 1 showed significant depletion of human CD34+ cells in the bone marrow (FIGS. 3A and B, FIG. 3A shown as % cells maintained and FIG. 3B shown as absolute cell count per femur) relative to the PBS control, 21 days following a single administration of the treatment regimen. Further, the results indicate that the bs-Ab 1 bispecific antibody from Example 1 showed significant depletion of human CD34+CD117+ cells in the bone marrow (FIGS. 3C and 3D, FIG. 3C shown as % cells maintained and FIG. 3D shown as absolute cell count per femur) 21 days following a single administration of the treatment regimen.

### Example 5. Production of the CD117/CD3 Bispecific Antibodies, Bs-Ab-2 and Bs-Ab-3

CD3/CD117 bispecific antibodies bs-Ab-2 and bs-Ab-3 were engineered using antigen binding sequences described in Table 4 and using the “knob-in-hole” bispecific engineering technique. bs-Ab-2, having a CD117-binding arm and a CD3-binding arm, was prepared using the “knob-in-hole” heterodimerization technology as described below. The CD117-binding arm of bs-Ab-2 comprises the heavy chain variable region sequence set forth in SEQ ID NO: 13 and the light chain variable region sequence set forth in SEQ ID NO: 14, and was engineered to introduce the following Fc substitutions, T366Y and H435A. Using site-directed mutagenesis, the Fc region of the heavy chain sequence of the anti-CD117 antibody Ab85 (i.e., SEQ ID NO: 150 as described in US 2019/0153114 A1, the content of which is hereby expressly incorporated by reference in its entirety) was engineered to introduce the following substitutions, T366Y and H435A (amino acid positions refer to the Fc region according to the EU index). The anti-CD117 Ab85 variant, i.e., “Ab85 T366Y H435A,” was subsequently expressed.

The CD3-binding arm of bs-Ab-2 comprises the heavy chain variable region sequence as set forth in SEQ ID NO: 41 and the light chain variable region sequence as set forth in SEQ ID NO: 45, and was engineered to introduce the following Fc substitutions, Y407T and H435A.

The Fc region of the Ab2 (anti-CD3) antibody heavy chain sequence set forth SEQ ID NO: 49 was engineered to introduce the following substitutions, Y407T and H435A (amino acid positions refer to the Fc region according to the EU index). The anti-CD3 antibody variant, “anti-CD3 Y407T H435A,” was subsequently expressed. The Ab85 T366Y H435A parental antibody and anti-CD3 Y407T H435A parental antibody were then assembled using standard knob and hole techniques to produce the bispecific heterodimer, bs-Ab-2.

Another bispecific antibody, i.e., bs-Ab-3, having a CD117-binding arm and a CD3-binding arm was prepared using the “knob-in-hole” heterodimerization technology as described below. The CD117-binding arm of bs-Ab-3 comprises the heavy chain variable region sequence set forth in SEQ ID NO: 27 and the light chain variable region sequence set forth in SEQ ID NO: 28, and was engineered to introduce the following Fc substitutions, T366Y H453A. Using site-directed mutagenesis, the Fc region of the heavy chain sequence of the anti-CD117 antibody Ab67 (i.e., SEQ ID NO: 152 as described in US 2019-0144558 A1, the content of which is hereby expressly incorporated by reference in its entirety) was engineered to introduce the following substitutions, T366Y and H435A (amino acid positions refer to the Fc region according to the EU index). The anti-CD117 Ab67 variant, i.e., “Ab67 T366Y H453A,” was subsequently expressed.

In addition, the CD3-binding arm of bs-Ab-3 comprises the heavy chain variable region sequence as set forth in SEQ ID NO: 41 and the light chain variable region sequence as set forth in SEQ ID NO: 45, and was engineered to introduce the following Fc substitutions, Y407T and H435A.

The Fc region of the Ab2 (anti-CD3) antibody heavy chain sequence set forth SEQ ID NO: 49 was engineered to introduce the following substitution, Y407T H435A (amino acid positions refer to the Fc region according to the EU index). The anti-CD3 antibody variant, “anti-CD3 Y407T H435A,” was subsequently expressed. The Ab67 T366Y H453A parental antibody and anti-CD3 Y407V H435A parental antibody were then assembled using standard knob and hole techniques to produce the bispecific heterodimer, bs-Ab-3.

In addition, three monospecific antibodies (i.e., an antibody having one binding arm and one non-targeting arm) were engineered for controls. A first monospecific antibody (i.e., an antibody having one binding arm and one non-targeting arm), Ab85-T366Y-H435A-Iso-Y407T-H435A, having a CD117-binding arm and an isotype (i.e., non-binding) arm, was prepared using the “knob-in-hole” heterodimerization technology. The CD117-binding arm of Ab85-T366Y-H435A-Iso-Y407T-H435A was prepared to include the heavy chain variable region sequence set forth in SEQ ID NO: 13 and the light chain variable region sequence set forth in SEQ ID NO: 14, and was engineered to introduce the following Fc substitutions, T366Y and H435A. In addition, the isotype-binding arm of Ab85-T366Y-H435A-Iso-Y407T-H435A was prepared to include the heavy chain variable region sequence of an isotype antibody, and was engineered to introduce the following Fc substitutions, Y407T and H435A.

A second monospecific antibody (i.e., an antibody having one binding arm and one non-targeting arm), Ab67-T366Y-H435A-Iso-Y407T-H435A, having a CD117-binding arm and an isotype (i.e., non-binding) arm, was prepared using the “knob-in-hole” heterodimerization technology. The CD117-binding arm of Ab67-T366Y-H435A-Iso-Y407T-H435A was prepared to include the heavy chain variable region sequence set forth in SEQ ID NO: 27 and the light chain variable region sequence set forth in SEQ ID NO: 28, and was engineered to introduce the following Fc substitutions, T366Y and H435A. In addition, the isotype-binding arm of Ab67-T366Y-H435A-Iso-Y407T-H435A was prepared to include the heavy chain variable region sequence of an isotype antibody, and was engineered to introduce the following Fc substitutions, Y407T and H435A.

A third monospecific antibody (i.e., an antibody having one binding arm and one non-targeting arm), Ab2-Y407T-H435A-Iso-T366Y-H435A, having a CD3-binding arm and an isotype (i.e., non-binding) arm, was prepared using the “knob-in-hole” heterodimerization technology. The CD3-binding arm of Ab2-Y407T-H435A-Iso-T366Y-H435A was prepared to include the heavy chain variable region sequence set forth in SEQ ID NO: 41 and the light chain variable region sequence set forth in SEQ ID NO: 45, and was engineered to introduce the following Fc substitutions, T366Y and H435A. In addition, the isotype-binding arm of Ab2-Y407T-H435A-Iso-T366Y-H435A was prepared to include the heavy chain variable region sequence of an isotype antibody, and was engineered to introduce the following Fc substitutions, Y407T and H435A.

The stability of the bs-Ab-2 bispecific antibody, the bs-Ab-3 bispecific antibody, and the three control antibodies (i.e., the Ab85-T366Y-H435A-Iso-Y407T-H435A antibody, the Ab67-T366Y-H435A-isotype-Y407T-H435A antibody and the anti-CD3-Y407T-H435A-Iso-T366Y-H435A antibody) were evaluated for stability and found to be stable in accordance with the tests performed for the bispecific antibodies, including no observable aggregation.

### Example 6. In Vitro Cell Killing Assay Using an Anti-CD117/Anti-CD3 Bispecific Antibody

For in vitro cell killing assays using human hematopoietic stem cells, human bone marrow-derived CD34+ cells were cultured for six days in the presence of either the bs-Ab-2 bispecific antibody from Example 5, the bs-Ab-3 bispecific antibody from Example 5, a combination of the Ab85-T366Y-H435A-Iso-Y407T-H435A antibody (from Example 5) and anti-CD3-Y407T-H435A-Iso-T366Y-H435A antibody (from Example 5) and a combination of the Ab67-T366Y-H435A-isotype-Y407T-H435A antibody (from Example 5) and the anti-CD3-Y407T-H435A-Iso-T366Y-H435A antibody (from Example 5). Cell viability was measured using flow cytometry.

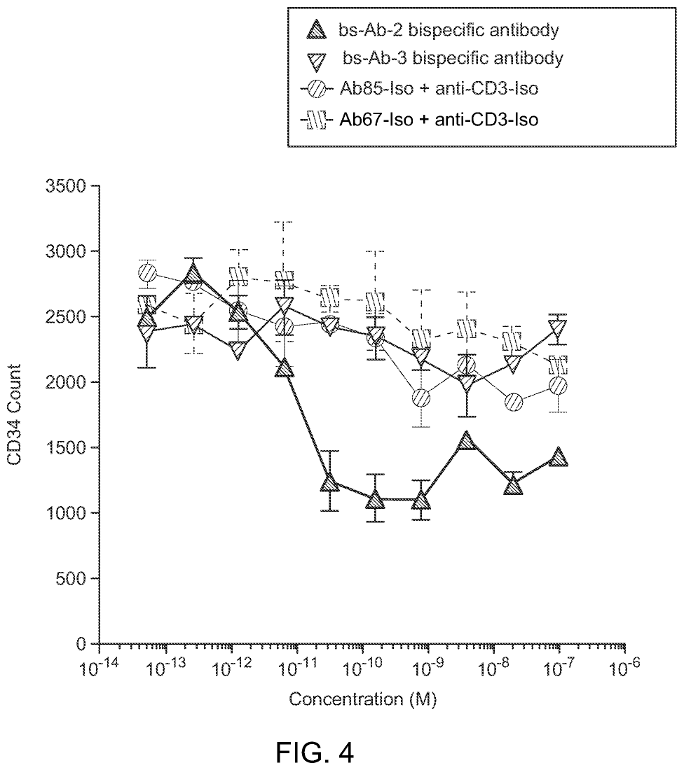

The results in FIG. 4 indicate that at day 6, the bs-Ab-2 bispecific antibody from Example 5 was effective at killing primary human CD34+ bone marrow cells in vitro (FIG. 4; IC₅₀=6.4 pM) in comparison to either the combination of the Ab85-T366Y-H435A-Iso-Y407T-H435A monospecific antibody and anti-CD3-Y407T-H435A-Iso-T366Y-H435A antibody and a combination of the Ab67-T366Y-H435A-isotype-Y407T-H435A antibody and the anti-CD3-Y407T-H435A-Iso-T366Y-H435A monospecific antibody (FIG. 4; no significant depletion of the CD34+ cells was observed). These data also demonstrated that the bs-Ab-2 bispecific antibody from Example 5 is more efficacious than the bs-Ab-3 bispecific antibody from Example 5 (FIG. 4). The difference in efficacy may be due to proximity of the bs-Ab-2 epitope to the cell membrane (the epitope of the Ab85 antibody is described in WO 2020/219770, which is hereby incorporated herein by reference in its entirety) compared to the proximity of the bs-Ab-3 epitope to the cell membrane (the epitope of the Ab67 antibody is described in WO 2020/219748, which is hereby incorporated herein by reference in its entirety).

The % efficacy values at 1 nM of the bispecific antibody are set forth in Table 3 below.

TABLE 3

| Molecule | % Efficacy (at 1 nM) |
|---|---|
| bs-Ab-2 bispecific antibody | 57 |
| bs-Ab-3 bispecific antibody | 16 |

TABLE 4
SEQUENCE SUMMARY

| Sequence | | |
|---|---|---|
| Identifier | Description | Amino Acid Sequence |
| SEQ ID NO: 1 | CK6 CDR-H1 | SYWIG |
| SEQ ID NO: 2 | CK6 CDR-H2 | HYPGDSDTRYSPSFQG |
| SEQ ID NO: 3 | CK6 CDR-H3 | HGRGYNGYEGAFDI |
| SEQ ID NO: 4 | CK6 CDR-L1 | RASQGISSALA |
| SEQ ID NO: 5 | CK6 CDR-L2 | DASSLES |
| SEQ ID NO: 6 | CK6 CDR-L3 | CQQFNSYPLT |
| SEQ ID NO: 7 | Ab85 CDR-H1 | NYWIG |
| SEQ ID NO: 8 | Ab85 CDR-H2 | HNPRDSDTRYRPSFQG |
| SEQ ID NO: 9 | Ab85 CDR-H3 | HGRGYEGYEGAFDI |
| SEQ ID | Ab85 CDR-L1 | RSSQGIRSDLG |
| NO: 10 | | |
| SEQ ID | Ab85 CDR-L2 | DASNLET |
| NO: 11 | | |
| SEQ ID | Ab85 CDR-L3 | QQANGFPLT |
| NO: 12 | | |
| SEQ ID | Heavy chain | EVQLVQSGAEVKKPGESLKISCKGSGYSFTNYWIG |
| NO: 13 | variable region of | WVRQMPGKGLEWMAIINPRDSDTRYRPSFQGQVTI |
| | Ab 85 | SADKSISTAYLQWSSLKASDTAMYYCARHGRGYEG |
| | | YEGAFDIWGQGTLVTVSS |
| SEQ ID | Light chain variable | DIQMTQSPSSLSASVGDRVTITCRSSQGIRSDLGWY |
| NO: 14 | region of Ab 85 | QQKPGKAPKLLIYDASNLETGVPSRFSGSGSGTDFT |
| | | LTISSLQPEDFATYYCQQANGFPLTFGGGTKVEIK |
| SEQ ID | Heavy chain | ASTKGPSVFPLAPSSKSTSGGTAALGCLVKDYFPEP |
| NO: 15 | constant region | VTVSWNSGALTSGVHTFPAVLQSSGLYSLSSVVTVP |
| | (Wild type (WT)) | SSSLGTQTYICNVNHKPSNTKVDKKVEPKSCDKTHT |
| | | CPPCPAPELLGGPSVFLFPPKPKDTLMISRTPEVTC |
| | | VVVDVSHEDPEVKFNWYVDGVEVHNAKTKPREEQY |
| | | NSTYRVVSVLTVLHQDWLNGKEYKCKVSNKALPAPI |
| | | EKTISKAKGQPREPQVYTLPPSRDELTKNQVSLTCL |
| | | VKGFYPSDIAVEWESNGQPENNYKTTPPVLDSDGS |
| | | FFLYSKLTVDKSRWQQGNVFSCSVMHEALHNHYTQ |
| | | KSLSLSPGK |
| SEQ ID | Heavy chain | ASTKGPSVFPLAPSSKSTSGGTAALGCLVKDYFPEP |
| NO: 16 | constant region | VTVSWNSGALTSGVHTFPAVLQSSGLYSLSSVVTVP |
| | with L234A, L235A | SSSLGTQTYICNVNHKPSNTKVDKKVEPKSCDKTHT |
| | (LALA) mutations | CPPCPAPEAAGGPSVFLFPPKPKDTLMISRTPEVTC |
| | (mutations in bold)* | VWDVSHEDPEVKFNWYVDGVEVHNAKTKPREEQY |
| | | NSTYRVVSVLTVLHQDWLNGKEYKCKVSNKALPAPI |
| | | EKTISKAKGQPREPQVYTLPPSRDELTKNQVSLTCL |
| | | VKGFYPSDIAVEWESNGQPENNYKTTPPVLDSDGS |
| | | FFLYSKLTVDKSRWQQGNVFSCSVMHEALHNHYTQ |
| | | KSLSLSPGK |
| SEQ ID | Heavy chain | ASTKGPSVFPLAPSSKSTSGGTAALGCLVKDYFPEP |
| NO: 17 | constant region | VTVSWNSGALTSGVHTFPAVLQSSGLYSLSSVVTVP |
| | with D265C | SSSLGTQTYICNVNHKPSNTKVDKKVEPKSCDKTHT |
| | mutation | CPPCPAPELLGGPSVFLFPPKPKDTLMISRTPEVTCV |
| | (mutation in bold)* | VVCVSHEDPEVKFNWYVDGVEVHNAKTKPREEQYN |
| | | STYRVVSVLTVLHQDWLNGKEYKCKVSNKALPAPIE |
| | | KTISKAKGQPREPQVYTLPPSRDELTKNQVSLTCLV |
| | | KGFYPSDIAVEWESNGQPENNYKTTPPVLDSDGSFF |
| | | LYSKLTVDKSRWQQGNVFSCSVMHEALHNHYTQKS |
| | | LSLSPGK |
| SEQ ID | Heavy chain | ASTKGPSVFPLAPSSKSTSGGTAALGCLVKDYFPEP |
| NO: 18 | constant region | VTVSWNSGALTSGVHTFPAVLQSSGLYSLSSVVTVP |
| | with H435A | SSSLGTQTYICNVNHKPSNTKVDKKVEPKSCDKTHT |
| | mutation | CPPCPAPELLGGPSVFLFPPKPKDTLMISRTPEVTCV |
| | (mutation in bold)* | VVDVSHEDPEVKFNWYVDGVEVHNAKTKPREEQYN |
| | | STYRVVSVLTVLHQDWLNGKEYKCKVSNKALPAPIE |
| | | KTISKAKGQPREPQVYTLPPSRDELTKNQVSLTCLV |
| | | KGFYPSDIAVEWESNGQPENNYKTTPPVLDSDGSFF |
| | | LYSKLTVDKSRWQQGNVFSCSVMHEALHNAYTQKS |
| | | LSLSPGK |
| SEQ ID | Heavy chain | ASTKGPSVFPLAPSSKSTSGGTAALGCLVKDYFPEP |
| NO: 19 | constant region: | VTVSWNSGALTSGVHTFPAVLQSSGLYSLSSVVTVP |
| | modified Fc region | SSSLGTQTYICNVNHKPSNTKVDKKVEPKSCDKTHT |
| | with L234A, L235A, | CPPCPAPEAAGGPSVFLFPPKPKDTLMISRTPEVTC |
| | D265C mutations | VVVCVSHEDPEVKFNWYVDGVEVHNAKTKPREEQY |
| | (mutations in bold)* | NSTYRVVSVLTVLHQDWLNGKEYKCKVSNKALPAPI |
| | | EKTISKAKGQPREPQVYTLPPSRDELTKNQVSLTCL |
| | | VKGFYPSDIAVEWESNGQPENNYKTTPPVLDSDGS |
| | | FFLYSKLTVDKSRWQQGNVFSCSVMHEALHNHYTQ |
| | | KS LSLSPGK |
| SEQ ID | Heavy chain | ASTKGPSVFPLAPSSKSTSGGTAALGCLVKDYFPEP |
| NO: 20 | constant region: | VTVSWNSGALTSGVHTFPAVLQSSGLYSLSSVVTVP |
| | modified Fc region | SSSLGTQTYICNVNHKPSNTKVDKKVEPKSCDKTHT |
| | with L234A, L235A, | CPPCPAPEAAGGPSVFLFPPKPKDTLMISRTPEVTC |
| | D265C, H435A | VVVCVSHEDPEVKFNWYVDGVEVHNAKTKPREEQY |
| | mutations | NSTYRVVSVLTVLHQDWLNGKEYKCKVSNKALPAPI |
| | (mutations in bold)* | EKTISKAKGQPREPQVYTLPPSRDELTKNQVSLTCL |
| | | VKGFYPSDIAVEWESNGQPENNYKTTPPVLDSDGS |
| | | FFLYSKLTVDKSRWQQGNVFSCSVMHEALHNAYTQ |
| | | KS LSLSPGK |
| SEQ ID | Ab67 CDR-H1 | FTFSDADMD |
| NO: 21 | | |
| SEQ ID | Ab67 CDR-H2 | RTRNKAGSYTTEYAASVKG |
| NO: 22 | | |
| SEQ ID | Ab67 CDR-H3 | AREPKYWIDFDL |
| NO: 23 | | |
| SEQ ID | Ab67 CDR-L1 | RASQSISSYLN |
| NO: 24 | | |
| SEQ ID | Ab67 CDR-L2 | AASSLQS |
| NO: 25 | | |
| SEQ ID | Ab67 CDR-L3 | QQSYIAPYT |
| NO: 26 | | |
| SEQ ID | Heavy chain | EVQLVESGGGLVQPGGSLRLSCAASGFTFSDADM |
| NO: 27 | variable region of | DWVRQAPGKGLEWVGRTRNKAGSYTTEYAASVKG |
| | Ab 67 | RFTISRDDSKNSLYLQMNSLKTEDTAVYYCAREPKY |
| | | WIDFDLWGRGTLVTVSS |
| SEQ ID | Light chain | DIQMTQSPSSLSASVGDRVTITCRASQSISSYLNWY |
| NO: 28 | variable region of | QQKPGKAPKLLIYAASSLQSGVPSRFSGSGSGTDFT |
| | Ab 67 | LTISSLQPEDFATYYCQQSYIAPYTFGGGTKVEIK |
| SEQ ID | CK6 heavy chain | QVQLVQSGAAVKKPGESLKISCKGSGYRFTSYWIG |
| NO: 29 | variable region | WVRQMPGKGLEWMGIIYPGDSDTRYSPSFQGQVTI |
| | amino acid | SAGKSISTAYLQWSSLKASDTAMYYCARHGRGYNG |
| | sequence (CDRs | YEGAFDIWGQGTMVTVSS |
| | bold) | |
| SEQ ID | CK6 light chain | AIQLTQSPSSLSASVGDRVTITCRASQGISSALAWY |
| NO: 30 | variable region | QQKPGKAPKLLIYDASSLESGVPSRFSGSGSGTD |
| | amino acid | FTLTISSLQPEDFATYYCQQFNSYPLTFGGGTKVEIK |
| | sequence (CDRs | |
| | bold) | |
| SEQ ID | human CD3 | agtctagctg ctgcacaggc tggctggctg gctggctgct |
| NO: 31 | gamma nucleic | aagggctgct ccacgctttt gccggaggac agagactgac |
| | acid sequence | atggaacagg ggaagggcct ggctgtcctc atcctggcta tcattcttct |
| | | tcaaggtact ttggcccagt caatcaaagg aaaccacttg |
| | | gttaaggtgt atgactatca agaagatggt tcggtacttc tgacttgtga |
| | | tgcagaagcc aaaaatatca catggtttaa agatgggaag |
| | | atgatcggct tcctaactga agataaaaaa aaatggaatc |
| | | tgggaagtaa tgccaaggac cctcgaggga tgtatcagtg |
| | | taaaggatca cagaacaagt caaaaccact ccaagtgtat |
| | | tacagaatgt gtcagaactg cattgaacta aatgcagcca |
| | | ccatatctgg ctttctcttt gctgaaatcg tcagcatttt cgtccttgct |
| | | gttggggtct acttcattgc tggacaggat ggagttcgcc |
| | | agtcgagagc ttcagacaag cagactctgt tgcccaatga |
| | | ccagctctac cagcccctca aggatcgaga agatgaccag |
| | | tacagccacc ttcaaggaaa ccagttgagg aggaattgaa |
| | | ctcaggactc agagtagtcc aggtgttctc ctcctattca gttcccagaa |
| | | tcaaagcaat gcattttgga aagctcctag cagagagact |
| | | ttcagcccta aatctagact caaggttccc agagatgaca |
| | | aatggagaag aaaggccatc agagcaaatt tgggggtttc |
| | | tcaaataaaa taaaaataaa aacaaatact gtgtttcaga |
| | | agcgccacct attggggaaa attgtaaaag aaaaatgaaa |
| | | agatcaaata accccctgga tttgaatata attttttgtg ttgtaatttt |
| | | tatttcgttt ttgtataggt tataattcac atggctcaaa tattcagtga |
| | | aagctctccc tccaccgcca tcccctgcta cccagtgacc |
| | | ctgttgccct cttcagagac aaattagttt ctcttttttt |
| | | tttttttttt tttttttttg |
| | | agacagtctg gctctgtcac ccaggctgaa atgcagtggc |
| | | accatctcgg ctcactgcaa cctctgcctc ctgggttcaa gcgattctcc |
| | | tgcctcagcc tcccgggcag ctgggattac aggcacacac |
| | | taccacacct ggctaatttt tgtattttta gtagagacag ggttttgctc |
| | | tgttggccaa gctggtctcg aactcctgac ctcaagtgat |
| | | ccgcccgcct c |
| SEQ ID | human CD3 | MEQGKGLAVLILAIILLQGTLAQSIKGNHLVKVYDYQE |
| NO: 32 | gamma amino acid | DGSVLLTCDAEAKNITWFKDGKMIGFLTEDKKKWNL |
| | sequence | GSNAKDPRGMYQCKGSQNKSKPLQVYYRMCQNCI |
| | | ELNAATISGFLFAEIVSIFVLAVGVYFIAGQDGVRQSR |
| | | ASDKQTLLPNDQLYQPLKDREDDQYSHLQGNQLRR |
| | | N |
| SEQ ID | human CD3 delta | agagaagcag acatcttcta gttcctcccc cactctcctc tttccggtac |
| NO: 33 | nucleic acid | ctgtgagtca gctaggggag ggcagctctc acccaggctg |
| | sequence | atagttcggt gacctggctt tatctactgg atgagttccg ctgggagatg |
| | | gaacatagca cgtttctctc tggcctggta ctggctaccc ttctctcgca |
| | | agtgagcccc ttcaagatac ctatagagga acttgaggac |
| | | agagtgtttg tgaattgcaa taccagcatc acatgggtag |
| | | agggaacggt gggaacactg ctctcagaca ttacaagact |
| | | ggacctggga aaacgcatcc tggacccacg aggaatatat |
| | | aggtgtaatg ggacagatat atacaaggac aaagaatcta |
| | | ccgtgcaagt tcattatcga atgtgccaga gctgtgtgga |
| | | gctggatcca gccaccgtgg ctggcatcat tgtcactgat |
| | | gtcattgcca ctctgctcct tgctttggga gtcttctgct ttgctggaca |
| | | tgagactgga aggctgtctg gggctgccga cacacaagct |
| | | ctgttgagga atgaccaggt ctatcagccc ctccgagatc |
| | | gagatgatgc tcagtacagc caccttggag gaaactgggc |
| | | tcggaacaag tgaacctgag actggtggct tctagaagca |
| | | gccattacca actgtacctt cccttcttgc tcagccaata aatatatcct |
| | | ctttcactca gaaaaaaaaa aaaaaaaaaa aaaaaaaaaa a |
| SEQ ID | human CD3 delta | MEHSTFLSGLVLATLLSQVSPFKIPIEELEDRVFVNC |
| NO: 34 | amino acid | NTSITWVEGTVGTLLSDITRLDLGKRILDPRGIYRCN |
| | sequence | GTDIYKDKESTVQVHYRMCQSCVELDPATVAGIIVTD |
| | | VIATLLLALGVFCFAGHETGRLSGAADTQALLRNDQV |
| | | YQPLRDRDDAQYSHLGGNWARNK |
| SEQ ID | human CD3 epsilon | tattgtcaga gtcctcttgt ttggccttct aggaaggctg tgggacccag |
| NO: 35 | nucleic acid | ctttcttcaa ccagtccagg tggaggcctc tgccttgaac gtttccaagt |
| | sequence | gaggtaaaac ccgcaggccc agaggcctct ctacttcctg |
| | | tgtggggttc agaaaccctc ctcccctccc agcctcaggt |
| | | gcctgcttca gaaaatgaag tagtaagtct gctggcctcc |
| | | gccatcttag taaagtaaca gtcccatgaa acaaagatgc |
| | | agtcgggcac tcactggaga gttctgggcc tctgcctctt atcagttggc |
| | | gtttgggggc aagatggtaa tgaagaaatg ggtggtatta |
| | | cacagacacc atataaagtc tccatctctg gaaccacagt |
| | | aatattgaca tgccctcagt atcctggatc tgaaatacta |
| | | tggcaacaca atgataaaaa cataggcggt gatgaggatg |
| | | ataaaaacat aggcagtgat gaggatcacc tgtcactgaa |
| | | ggaattttca gaattggagc aaagtggtta ttatgtctgc taccccagag |
| | | gaagcaaacc agaagatgcg aacttttatc tctacctgag |
| | | ggcaagagtg tgtgagaact gcatggagat ggatgtgatg |
| | | tcggtggcca caattgtcat agtggacatc tgcatcactg |
| | | ggggcttgct gctgctggtt tactactgga gcaagaatag |
| | | aaaggccaag gccaagcctg tgacacgagg agcgggtgct |
| | | ggcggcaggc aaaggggaca aaacaaggag aggccaccac |
| | | ctgttcccaa cccagactat gagcccatcc ggaaaggcca |
| | | gcgggacctg tattctggcc tgaatcagag acgcatctga |
| | | ccctctggag aacactgcct cccgctggcc caggtctcct |
| | | ctccagtccc cctgcgactc cctgtttcct gggctagtct tggaccccac |
| | | gagagagaat cgttcctcag cctcatggtg aactcgcgcc |
| | | ctccagcctg atcccccgct ccctcctccc tgccttctct gctggtaccc |
| | | agtcctaaaa tattgctgct tcctcttcct ttgaagcatc atcagtagtc |
| | | acaccctcac agctggcctg ccctcttgcc aggatattta tttgtgctat |
| | | tcactccctt ccctttggat gtaacttctc cgttcagttc cctccttttc |
| | | ttgcatgtaa gttgtccccc atcccaaagt attccatcta cttttctatc |
| | | gccgtcccct tttgcagccc tctctgggga tggactgggt aaatgttgac |
| | | agaggccctg ccccgttcac agatcctggc cctgagccag |
| | | ccctgtgctc ctccctcccc caacactccc taccaacccc |
| | | ctaatcccct actccctcca ccccccctcc actgtaggcc |
| | | actggatggt catttgcatc tccgtaaatg tgctctgctc ctcagctgag |
| | | agagaaaaaa ataaactgta tttggctgca agaaaaaaaa |
| | | aaaaaaaaaa aaaa |
| SEQ ID | human CD3 epsilon | MQSGTHWRVLGLCLLSVGVWGQDGNEEMGGITQT |
| NO: 36 | amino acid | PYKVSISGTTVILTCPQYPGSEILWQHNDKNIGGDED |
| | sequence | DKNIGSDEDHLSLKEFSELEQSGYYVCYPRGSKPED |
| | | ANFYLYLRARVCENCMEMDVMSVATIVIVDICITGGL |
| | | LLLVYYWSKNRKAKAKPVTRGAGAGGRQRGQNKE |
| | | RPPPVPNPDYEPIRKGQRDLYSGLNQRRI |
| SEQ ID | Heavy chain | EVQLVESGGGLVQPGGSLRLSCAASGFTFNSYAMN |
| NO: 37 | variable region of a | WVRQAPGKGLEWVARIRSKYNNYATYYADSVKGRF |
| | CD3 binding | TISRDDSKNTAYLQMNSLKTEDTAVYYCVRHGNFGN |
| | domain | SYVSWWAYWGQGTLVTVSS |
| SEQ ID | Light chain | QTVVTQEPSLTVSPGGTVTLTCGSSTGAVTSGNYPN |
| NO: 38 | variable region of a | WVQQKPGQAPRGLIGGTKFLAPGTPARFSGSLLGG |
| | CD3 binding | KAALTLSGVQPEDEAEYYCVLWYSNRWVFGGGTKL |
| | domain | TVL |
| SEQ ID NO: | Ab1 (anti-CD3) | EVQLVESGGGLVQPGKSLKLSCEASGFTFSGYGMH |
| 39 | antibody heavy | WVRQAPGRGLESVAYITSSSINIKYADAVKGRFTVS |
| | chain | RDNAKNLLFLQMNILKSEDTAMYYCARFDWDKNYWG |
| | | QGTMVTVSSASTKGPSVFPLAPSSKSTSGGTAALG |
| | | CLVKDYFPEPVTVSWNSGALTSGVHTFPAVLQSSGL |
| | | YSLSSVVTVPSSSLGTQTYICNVNHKPSNTKVDKKV |
| | | EPKSCDKTHTCPPCPAPEAAGGPSVFLFPPKPKDTL |
| | | MISRTPEVTCVVVDVSHEDPEVKFNWYVDGVEVHN |
| | | AKTKPREEQYNSTYRVVSVLTVLHQDWLNGKEYKCK |
| | | VSNKALPAPIEKTISKAKGQPREPQVYTLPPSREEM |
| | | TKNQVSLSCAVKGFYPSDIAVEWESNGQPENNYKT |
| | | TPPVLDSDGSFFLVSKLTVDKSRWQQGNVFSCSVM |
| | | HEALHNHYTQKSLSLSPGK |
| SEQ ID NO: | Ab1 (anti-CD3) | DIQMTQSPSSLPASLGDRVTINCQASQDISNYLNWY |
| 40 | antibody light | QQKPGKAPKLLIYYTNKLADGVPSRFSGSGSGRDSS |
| | chain | FTISSLESEDIGSYYCQQYYNYPWTFGPGTKLEIKRT |
| | | VAAPSVFIFPPSDEQLKSGTASVVCLLNNFYPREAKV |
| | | QWKVDNALQSGNSQESVTEQDSKDSTYSLSSTLTL |
| | | SKADYEKHKVYACEVTHQGLSSPVTKSFNRGEC |
| SEQ ID NO: | Ab2 (anti-CD3) | EVQLVQSGAEVKKPGASVKVSCKASGYTFTNYYIH |
| 41 | antibody heavy | WVRQAPGQGLEWIGWIYPGDGNTKYNEKFKGRATL |
| | chain variable | TADTSTSTAYLELSSLRSEDTAVYYCARDSYSNYYF |
| | region (CDRs | DYWGQGTLVTVSS |
| | underlined in bold; | |
| | IMGT naming | |
| | convention) | |
| SEQ ID NO: | Ab2-HC CDR1 | GYTFTNYY |
| 42 | | |
| SEQ ID NO: | Ab2-HC CDR2 | IYPGDGNT |
| 43 | | |
| SEQ ID NO: | Ab2-HC CDR3 | ARDSYSNYYFDY |
| 44 | | |
| SEQ ID NO: | Ab2 (anti-CD3) | DIVMTOSPDSLAVSLGERATINCKSSQSLLNSRTRK |
| 45 | antibody light | NYLAWYQQKPGQPPKLLIYWASTRESGVPDRFSGS |
| | chain variable | GSGTDFTLTISSLQAEDVAVYYCTQSFILRTFGQG |
| | region (CDRs | TKVEIK |
| | underlined in bold; | |
| | IMGT naming | |
| | convention) | |
| SEQ ID NO: | Ab2-LC CDR1 | QSLLNSRTRKNY |
| 46 | | |
| SEQ ID NO: | Ab2-LC CDR2 | WAS |
| 47 | | |
| SEQ ID NO: | Ab2-LC CDR3 | TQSFILRT |
| 48 | | |
| SEQ ID NO: | Ab2 (anti-CD3) | EVQLVQSGAEVKKPGASVKVSCKASGYTFTNYYIH |
| 49 | antibody heavy | WVRQAPGQGLEWIGWIYPGDGNTKYNEKFKGRATL |
| | chain | TADTSTSTAYLELSSLRSEDTAVYYCARDSYSNYYF |
| | | DYWGQGTLVTVSSASTKGPSVFPLAPSSKSTSGGT |
| | | AALGCLVKDYFPEPVTVSWNSGALTSGVHTFPAVLQ |
| | | SSGLYSLSSVVTVPSSSLGTQTYICNVNHKPSNTKV |
| | | DKKVEPKSCDKTHTCPPCPAPELLGGPSVFLFPPKP |
| | | KDTLMISRTPEVTCVVVDVSHEDPEVKFNWYVDGVE |
| | | VHNAKTKPREEQYGSTYRVVSVLTVLHQDWLNGKE |
| | | YKCKVSNKALPAPIEKTISKAKGQPREPQVYTLPPSR |
| | | EEMTKNQVSLSCAVKGFYPSDIAVEWESNGQPENN |
| | | YKTTPPVLDSDGSFFLVSKLTVDKSRWQQGNVFSC |
| | | SVMHEALHNHYTQKSLSLSPGK |
| SEQ ID NO: | Ab2 (anti-CD3) | DIVMTQSPDSLAVSLGERATINCKSSQSLLNSRTRKN |
| 50 | antibody light | YLAWYQQKPGQPPKLLIYWASTRESGVPDRFSGSG |
| | chain | SGTDFTLTISSLQAEDVAVYYCTQSFILRTFGQGTKV |
| | | EIKRTVAAPSVFIFPPSDEQLKSGTASVVCLLNNFYP |
| | | REAKVQWKVDNALQSGNSQESVTEQDSKDSTYSLS |
| | | STLTLSKADYEKHKVYACEVTHQGLSSPVTKSFNRG |
| | | EC |
| SEQ ID NO: | CDR-H1 of anti- | IYAMN |
| 51 | CD3 antibody F6A | |
| SEQ ID NO: | CDR-H2 of anti- | RIRSKYNNYATYYADSVKS |
| 52 | CD3 antibody F6A | |
| SEQ ID NO: | CDR-H3 of anti- | HGNFGNSYVSFFAY |
| 53 | CD3 antibody F6A | |
| SEQ ID NO: | VH of anti-CD3 | EVQLVESGGGLVQPGGSLKLSCAASGFTFNIYAMN |
| 54 | antibody F6A | WVRQAPGKGLEWVARIRSKYNNYATYYADSVKSRF |
| | | TISRDDSKNTAYLQMNNLKTEDTAVYYCVRHGNFGN |
| | | SYVSFFAYWGQGTLVTVSS |
| SEQ ID NO: | VL of anti-CD3 | QTVVTQEPSLTVSPGGTVTLTCGSSTGAVTSGYYPN |
| 55 | antibody F6A | WVQQKPGQAPRGLIGGTKFLAPGTPARFSGSLLGG |
| | | KAALTLSGVQPEDEAEYYCALWYSNRWVFGGGTKL |
| | | TVL |
| SEQ ID NO: | VH of anti-CD3 | EVQLLESGGGLVQPGGSLKLSCAASGFTFNIYAMN |
| 56 | antibody F6A | WVRQAPGKGLEWVARIRSKYNNYATYYADSVKSRF |
| | | TISRDDSKNTAYLQMNNLKTEDTAVYYCVRHGNFGN |
| | | SYVSFFAYWGQGTLVTVSS |
| SEQ ID NO: | VL of anti-CD3 | ELVVTQEPSLTVSPGGTVTLTCGSSTGAVTSGYYPN |
| 57 | antibody F6A | WVQQKPGQAPRGLIGGTKFLAPGTPARFSGSLLGG |
| | | KAALTLSGVQPEDEAEYYCALWYSNRWVFGGGTKL |
| | | TVL |
| SEQ ID NO: | VH-VL of anti-CD3 | EVQLVESGGGLVQPGGSLKLSCAASGFTFNIYAMN |
| 58 | antibody F6A | WVRQAPGKGLEWVARIRSKYNNYATYYADSVKSRF |
| | | TISRDDSKNTAYLQMNNLKTEDTAVYYCVRHGNFGN |
| | | SYVSFFAYWGQGTLVTVSSGGGGSGGGGSGGGGS |
| | | QTVVTQEPSLTVSPGGTVTLTCGSSTGAVTSGYYPN |
| | | WVQQKPGQAPRGLIGGTKFLAPGTPARFSGSLLGG |
| | | KAALTLSGVQPEDEAEYYCALWYSNRWVFGGGTKL |
| | | TVL |
| SEQ ID NO: | VH-VL of anti-CD3 | EVQLLESGGGLVQPGGSLKLSCAASGFTFNIYAMN |
| 59 | antibody F6A | WVRQAPGKGLEWVARIRSKYNNYATYYADSVKSRF |
| | | TISRDDSKNTAYLQMNNLKTEDTAVYYCVRHGNFGN |
| | | SYVSFFAYWGQGTLVTVSSGGGGSGGGGSGGGGS |
| | | ELVVTQEPSLTVSPGGTVTLTCGSSTGAVTSGYYPN |
| | | WVQQKPGQAPRGLIGGTKFLAPGTPARFSGSLLGG |
| | | KAALTLSGVQPEDEAEYYCALWYSNRWVFGGGTKL |
| | | TVL |
| SEQ ID NO: | CDR-L1 of anti- | GSSTGAVTSGYYPN |
| 60 | CD3 antibody H2C | |
| SEQ ID NO: | CDR-L2 of anti- | GTKFLAP |
| 61 | CD3 antibody H2C | |
| SEQ ID NO: | CDR-L3 of anti- | ALWYSNRWV |
| 62 | CD3 antibody H2C | |
| SEQ ID NO: | CDR-H1 of anti- | KYAMN |
| 63 | CD3 antibody H2C | |
| SEQ ID NO: | CDR-H2 of anti- | RIRSKYNNYATYYADSVKD |
| 64 | CD3 antibody H2C | |
| SEQ ID NO: | CDR-H3 of anti- | HGNFGNSYISYWAY |
| 65 | CD3 antibody H2C | |
| SEQ ID NO: | VH of anti-CD3 | EVQLVESGGGLVQPGGSLKLSCAASGFTFNKYAMN |
| 66 | antibody H2C | WVRQAPGKGLEWVARIRSKYNNYATYYADSVKDRF |
| | | TISRDDSKNTAYLQMNNLKTEDTAVYYCVRHGNFGN |
| | | SYISYWAYWGQGTLVTVSS |
| SEQ ID NO: | VL of anti-CD3 | QTVVTQEPSLTVSPGGTVTLTCGSSTGAVTSGYYPN |
| 67 | antibody H2C | WVQQKPGQAPRGLIGGTKFLAPGTPARFSGSLLGG |
| | | KAALTLSGVQPEDEAEYYCALWYSNRWVFGGGTKL |
| | | TVL |
| SEQ ID NO: | VH of anti-CD3 | EVQLLESGGGLVQPGGSLKLSCAASGFTFNKYAMN |
| 68 | antibody H2C | WVRQAPGKGLEWVARIRSKYNNYATYYADSVKDRF |
| | | TISRDDSKNTAYLQMNNLKTEDTAVYYCVRHGNFGN |
| | | SYISYWAYWGQGTLVTVSS |
| SEQ ID NO: | VL of anti-CD3 | ELVVTQEPSLTVSPGGTVTLTCGSSTGAVTSGYYPN |
| 69 | antibody H2C | WVQQKPGQAPRGLIGGTKFLAPGTPARFSGSLLGG |
| | | KAALTLSGVQPEDEAEYYCALWYSNRWVFGGGTKL |
| | | TVL |
| SEQ ID NO: | VH-VL of anti-CD3 | EVQLVESGGGLVQPGGSLKLSCAASGFTFNKYAMN |
| 70 | antibody H2C | WVRQAPGKGLEWVARIRSKYNNYATYYADSVKDRF |
| | | TISRDDSKNTAYLQMNNLKTEDTAVYYCVRHGNFGN |
| | | SYISYWAYWGQGTLVTVSSGGGGSGGGGSGGGGS |
| | | QTVVTQEPSLTVSPGGTVTLTCGSSTGAVTSGYYPN |
| | | WVQQKPGQAPRGLIGGTKFLAPGTPARFSGSLLGG |
| | | KAALTLSGVQPEDEAEYYCALWYSNRWVFGGGTKL |
| | | TVL |
| SEQ ID NO: | VH-VL of anti-CD3 | EVQLLESGGGLVQPGGSLKLSCAASGFTFNKYAMN |
| 71 | antibody H2C | WVRQAPGKGLEWVARIRSKYNNYATYYADSVKDRF |
| | | TISRDDSKNTAYLQMNNLKTEDTAVYYCVRHGNFGN |
| | | SYISYWAYWGQGTLVTVSSGGGGSGGGGSGGGGS |
| | | ELVVTQEPSLTVSPGGTVTLTCGSSTGAVTSGYYPN |
| | | WVQQKPGQAPRGLIGGTKFLAPGTPARFSGSLLGG |
| | | KAALTLSGVQPEDEAEYYCALWYSNRWVFGGGTKL |
| | | TVL |
| SEQ ID NO: | CDR-H1 of anti- | SYAMN |
| 72 | CD3 antibody H1E | |
| SEQ ID NO: | CDR-H2 of anti- | RIRSKYNNYATYYADSVKG |
| 73 | CD3 antibody H1E | |
| SEQ ID NO: | CDR-H3 of anti- | HGNFGNSYLSFWAY |
| 74 | CD3 antibody H1E | |
| SEQ ID NO: | VH of anti-CD3 | EVQLVESGGGLEQPGGSLKLSCAASGFTFNSYAMN |
| 75 | antibody H1E | WVRQAPGKGLEWVARIRSKYNNYATYYADSVKGRF |
| | | TISRDDSKNTAYLQMNNLKTEDTAVYYCVRHGNFGN |
| | | SYLSFWAYWGQGTLVTVSS |
| SEQ ID NO: | VL of anti-CD3 | QTVVTQEPSLTVSPGGTVTLTCGSSTGAVTSGYYPN |
| 76 | antibody H1E | WVQQKPGQAPRGLIGGTKFLAPGTPARFSGSLLGG |
| | | KAALTLSGVQPEDEAEYYCALWYSNRWVFGGGTKL |
| | | TVL |
| SEQ ID NO: | VH of anti-CD3 | EVQLLESGGGLEQPGGSLKLSCAASGFTFNSYAMN |
| 77 | antibody H1E | WVRQAPGKGLEWVARIRSKYNNYATYYADSVKGRF |
| | | TISRDDSKNTAYLQMNNLKTEDTAVYYCVRHGNFGN |
| | | SYLSFWAYWGQGTLVTVSS |
| SEQ ID NO: | VL of anti-CD3 | ELVVTQEPSLTVSPGGTVTLTCGSSTGAVTSGYYPN |
| 78 | antibody H1E | WVQQKPGQAPRGLIGGTKFLAPGTPARFSGSLLGG |
| | | KAALTLSGVQPEDEAEYYCALWYSNRWVFGGGTKL |
| | | TVL |
| SEQ ID NO: | VH-VL of anti-CD3 | EVQLVESGGGLEQPGGSLKLSCAASGFTFNSYAMN |
| 79 | antibody H1E | WVRQAPGKGLEWVARIRSKYNNYATYYADSVKGRF |
| | | TISRDDSKNTAYLQMNNLKTEDTAVYYCVRHGNFGN |
| | | SYLSFWAYWGQGTLVTVSSGGGGSGGGGSGGGG |
| | | SQTVVTQEPSLTVSPGGTVTLTCGSSTGAVTSGYYP |
| | | NWVQQKPGQAPRGLIGGTKFLAPGTPARFSGSLLG |
| | | GKAALTLSGVQPEDEAEYYCALWYSNRWVFGGGTK |
| | | LTVL |
| SEQ ID NO: | VH-VL of anti-CD3 | EVQLLESGGGLEQPGGSLKLSCAASGFTFNSYAMN |
| 80 | antibody H1E | WVRQAPGKGLEWVARIRSKYNNYATYYADSVKGRF |
| | | TISRDDSKNTAYLQMNNLKTEDTAVYYCVRHGNFGN |
| | | SYLSFWAYWGQGTLVTVSSGGGGSGGGGSGGGG |
| | | SELVVTQEPSLTVSPGGTVTLTCGSSTGAVTSGYYP |
| | | NWVQQKPGQAPRGLIGGTKFLAPGTPARFSGSLLG |
| | | GKAALTLSGVQPEDEAEYYCALWYSNRWVFGGGTK |
| | | LTVL |
| SEQ ID NO: | CDR-H1 of anti- | RYAMN |
| 81 | CD3 antibody G4H | |
| SEQ ID NO: | CDR-H2 of anti- | RIRSKYNNYATYYADSVKG |
| 82 | CD3 antibody G4H | |
| SEQ ID NO: | CDR-H3 of anti- | HGNFGNSYLSYFAY |
| 83 | CD3 antibody G4H | |
| SEQ ID NO: | VH of anti-CD3 | EVQLVESGGGLVQPGGSLKLSCAASGFTFNRYAMN |
| 84 | antibody G4H | WVRQAPGKGLEWVARIRSKYNNYATYYADSVKGRF |
| | | TISRDDSKNTAYLQMNNLKTEDTAVYYCVRHGNFGN |
| | | SYLSYFAYWGQGTLVTVSS |
| SEQ ID NO: | VL of anti-CD3 | QTVVTQEPSLTVSPGGTVTLTCGSSTGAVTSGYYPN |
| 85 | antibody G4H | WVQQKPGQAPRGLIGGTKFLAPGTPARFSGSLLGG |
| | | KAALTLSGVQPEDEAEYYCALWYSNRWVFGGGTKL |
| | | TVL |
| SEQ ID NO: | VH of anti-CD3 | EVQLLESGGGLVQPGGSLKLSCAASGFTFNRYAMN |
| 86 | antibody G4H | WVRQAPGKGLEWVARIRSKYNNYATYYADSVKGRF |
| | | TISRDDSKNTAYLQMNNLKTEDTAVYYCVRHGNFGN |
| | | SYLSYFAYWGQGTLVTVSS |
| SEQ ID NO: | VL of anti-CD3 | ELVVTQEPSLTVSPGGTVTLTCGSSTGAVTSGYYPN |
| 87 | antibody G4H | WVQQKPGQAPRGLIGGTKFLAPGTPARFSGSLLGG |
| | | KAALTLSGVQPEDEAEYYCALWYSNRWVFGGGTKL |
| | | TVL |
| SEQ ID NO: | VH-VL of anti-CD3 | EVQLVESGGGLVQPGGSLKLSCAASGFTFNRYAMN |
| 88 | antibody G4H | WVRQAPGKGLEWVARIRSKYNNYATYYADSVKGRF |
| | | TISRDDSKNTAYLQMNNLKTEDTAVYYCVRHGNFGN |
| | | SYLSYFAYWGQGTLVTVSSGGGGSGGGGSGGGGS |
| | | QTVVTQEPSLTVSPGGTVTLTCGSSTGAVTSGYYPN |
| | | WVQQKPGQAPRGLIGGTKFLAPGTPARFSGSLLGG |
| | | KAALTLSGVQPEDEAEYYCALWYSNRWVFGGGTKL |
| | | TVL |
| SEQ ID NO: | VH-VL of anti-CD3 | EVQLLESGGGLVQPGGSLKLSCAASGFTFNRYAMN |
| 89 | antibody G4H | WVRQAPGKGLEWVARIRSKYNNYATYYADSVKGRF |
| | | TISRDDSKNTAYLQMNNLKTEDTAVYYCVRHGNFGN |
| | | SYLSYFAYWGQGTLVTVSSGGGGSGGGGSGGGGS |
| | | ELVVTQEPSLTVSPGGTVTLTCGSSTGAVTSGYYPN |
| | | WVQQKPGQAPRGLIGGTKFLAPGTPARFSGSLLGG |
| | | KAALTLSGVQPEDEAEYYCALWYSNRWVFGGGTKL |
| | | TVL |
| SEQ ID NO: | CDR-H1 of anti- | VYAMN |
| 90 | CD3 antibody A2J | |
| SEQ ID NO: | CDR-H2 of anti- | RIRSKYNNYATYYADSVKK |
| 91 | CD3 antibody A2J | |
| SEQ ID NO: | CDR-H3 of anti- | HGNFGNSYLSWWAY |
| 92 | CD3 antibody A2J | |
| SEQ ID NO: | VH of anti-CD3 | EVQLVESGGGLVQPGGSLKLSCAASGFTFNVYAMN |
| 93 | antibody A2J | WVRQAPGKGLEWVARIRSKYNNYATYYADSVKKRF |
| | | TISRDDSKNTAYLQMNNLKTEDTAVYYCVRHGNFGN |
| | | SYLSWWAYWGQGTLVTVSS |
| SEQ ID NO: | VL of anti-CD3 | QTVVTQEPSLTVSPGGTVTLTCRSSTGAVTSGYYPN |
| 94 | antibody A2J | WVQQKPGQAPRGLIGATDMRPSGTPARFSGSLLGG |
| | | KAALTLSGVQPEDEAEYYCALWYSNRWVFGGGTKL |
| | | TVL |
| SEQ ID NO: | VH of anti-CD3 | EVQLLESGGGLVQPGGSLKLSCAASGFTFNVYAMN |
| 95 | antibody A2J | WVRQAPGKGLEWVARIRSKYNNYATYYADSVKKRF |
| | | TISRDDSKNTAYLQMNNLKTEDTAVYYCVRHGNFGN |
| | | SYLSWWAYWGQGTLVTVSS |
| SEQ ID NO: | VL of anti-CD3 | ELVVTQEPSLTVSPGGTVTLTCRSSTGAVTSGYYPN |
| 96 | antibody A2J | WVQQKPGQAPRGLIGATDMRPSGTPARFSGSLLGG |
| | | KAALTLSGVQPEDEAEYYCALWYSNRWVFGGGTKL |
| | | TVL |
| SEQ ID NO: | VH-VL of anti-CD3 | EVQLVESGGGLVQPGGSLKLSCAASGFTFNVYAMN |
| 97 | antibody A2J | WVRQAPGKGLEWVARIRSKYNNYATYYADSVKKRF |
| | | TISRDDSKNTAYLQMNNLKTEDTAVYYCVRHGNFGN |
| | | SYLSWWAYWGQGTLVTVSSGGGGSGGGGSGGGG |
| | | SQTVVTQEPSLTVSPGGTVTLTCRSSTGAVTSGYYP |
| | | NWVQQKPGQAPRGLIGATDMRPSGTPARFSGSLLG |
| | | GKAALTLSGVQPEDEAEYYCALWYSNRWVFGGGTK |
| | | LTVL |
| SEQ ID NO: | VH-VL of anti-CD3 | EVQLLESGGGLVQPGGSLKLSCAASGFTFNVYAMN |
| 98 | antibody A2J | WVRQAPGKGLEWVARIRSKYNNYATYYADSVKKRF |
| | | TISRDDSKNTAYLQMNNLKTEDTAVYYCVRHGNFGN |
| | | SYLSWWAYWGQGTLVTVSSGGGGSGGGGSGGGG |
| | | SELVVTQEPSLTVSPGGTVTLTCRSSTGAVTSGYYP |
| | | NWVQQKPGQAPRGLIGATDMRPSGTPARFSGSLLG |
| | | GKAALTLSGVQPEDEAEYYCALWYSNRWVFGGGTK |
| | | LTVL |
| SEQ ID NO: | CDR-H1 of anti- | KYAMN |
| 99 | CD3 antibody E1L | |
| SEQ ID NO: | CDR-H2 of anti- | RIRSKYNNYATYYADSVKS |
| 100 | CD3 antibody E1L | |
| SEQ ID NO: | CDR-H3 of anti- | HGNFGNSYTSYYAY |
| 101 | CD3 antibody E1L | |
| SEQ ID NO: | VH of anti-CD3 | EVQLVESGGGLVQPGGSLKLSCAASGFTFNKYAMN |
| 102 | antibody E1L | WVRQAPGKGLEWVARIRSKYNNYATYYADSVKSRF |
| | | TISRDDSKNTAYLQMNNLKTEDTAVYYCVRHGNFGN |
| | | SYTSYYAYWGQGTLVTVSS |
| SEQ ID NO: | VL of anti-CD3 | QTVVTQEPSLTVSPGGTVTLTCGSSTGAVTSGYYPN |
| 103 | antibody E1L | WVQQKPGQAPRGLIGGTKFLAPGTPARFSGSLLGG |
| | | KAALTLSGVQPEDEAEYYCALWYSNRWVFGGGTKL |
| | | TVL |
| SEQ ID NO: | VH of anti-CD3 | EVQLLESGGGLVQPGGSLKLSCAASGFTFNKYAMN |
| 104 | antibody E1L | WVRQAPGKGLEWVARIRSKYNNYATYYADSVKSRF |
| | | TISRDDSKNTAYLQMNNLKTEDTAVYYCVRHGNFGN |
| | | SYTSYYAYWGQGTLVTVSS |
| SEQ ID NO: | VL of anti-CD3 | ELVVTQEPSLTVSPGGTVTLTCGSSTGAVTSGYYPN |
| 105 | antibody E1L | WVQQKPGQAPRGLIGGTKFLAPGTPARFSGSLLGG |
| | | KAALTLSGVQPEDEAEYYCALWYSNRWVFGGGTKL |
| | | TVL |
| SEQ ID NO: | VH-VL of anti-CD3 | EVQLVESGGGLVQPGGSLKLSCAASGFTFNKYAMN |
| 106 | antibody E1L | WVRQAPGKGLEWVARIRSKYNNYATYYADSVKSRF |
| | | TISRDDSKNTAYLQMNNLKTEDTAVYYCVRHGNFGN |
| | | SYTSYYAYWGQGTLVTVSSGGGGSGGGGSGGGG |
| | | SQTVVTQEPSLTVSPGGTVTLTCGSSTGAVTSGYYP |
| | | NWVQQKPGQAPRGLIGGTKFLAPGTPARFSGSLLG |
| | | GKAALTLSGVQPEDEAEYYCALWYSNRWVFGGGTK |
| | | LTVL |
| SEQ ID NO: | VH-VL of anti-CD3 | EVQLLESGGGLVQPGGSLKLSCAASGFTFNKYAMN |
| 107 | antibody E1L | WVRQAPGKGLEWVARIRSKYNNYATYYADSVKSRF |
| | | TISRDDSKNTAYLQMNNLKTEDTAVYYCVRHGNFGN |
| | | SYTSYYAYWGQGTLVTVSSGGGGSGGGGSGGGG |
| | | SELVVTQEPSLTVSPGGTVTLTCGSSTGAVTSGYYP |
| | | NWVQQKPGQAPRGLIGGTKFLAPGTPARFSGSLLG |
| | | GKAALTLSGVQPEDEAEYYCALWYSNRWVFGGGTK |
| | | LTVL |
| SEQ ID NO: | CDR-L1 of anti- | RSSTGAVTSGYYPN |
| 108 | CD3 antibody E2M | |
| SEQ ID NO: | CDR-L2 of anti- | ATDMRPS |
| 109 | CD3 antibody E2M | |
| SEQ ID NO: | CDR-L3 of anti- | ALWYSNRWV |
| 110 | CD3 antibody E2M | |
| SEQ ID NO: | CDR-H1 of anti- | GYAMN |
| 111 | CD3 antibody E2M | |
| SEQ ID NO: | CDR-H2 of anti- | RIRSKYNNYATYYADSVKE |
| 112 | CD3 antibody E2M | |
| SEQ ID NO: | CDR-H3 of anti- | HRNFGNSYLSWFAY |
| 113 | CD3 antibody E2M | |
| SEQ ID NO: | VH of anti-CD3 | EVQLVESGGGLVQPGGSLKLSCAASGFTFNGYAMN |
| 114 | antibody E2M | WVRQAPGKGLEWVARIRSKYNNYATYYADSVKERF |
| | | TISRDDSKNTAYLQMNNLKTEDTAVYYCVRHRNFGN |
| | | SYLSWFAYWGQGTLVTVSS |
| SEQ ID NO: | VL of anti-CD3 | QTVVTQEPSLTVSPGGTVTLTCRSSTGAVTSGYYPN |
| 115 | antibody E2M | WVQQKPGQAPRGLIGATDMRPSGTPARFSGSLLGG |
| | | KAALTLSGVQPEDEAEYYCALWYSNRWVFGGGTKL |
| | | TVL |
| SEQ ID NO: | VH of anti-CD3 | EVQLLESGGGLVQPGGSLKLSCAASGFTFNGYAMN |
| 116 | antibody E2M | WVRQAPGKGLEWVARIRSKYNNYATYYADSVKERF |
| | | TISRDDSKNTAYLQMNNLKTEDTAVYYCVRHRNFGN |
| | | SYLSWFAYWGQGTLVTVSS |
| SEQ ID NO: | VL of anti-CD3 | ELVVTQEPSLTVSPGGTVTLTCRSSTGAVTSGYYPN |
| 117 | antibody E2M | WVQQKPGQAPRGLIGATDMRPSGTPARFSGSLLGG |
| | | KAALTLSGVQPEDEAEYYCALWYSNRWVFGGGTKL |
| | | TVL |
| SEQ ID NO: | VH-VL of anti-CD3 | EVQLVESGGGLVQPGGSLKLSCAASGFTFNGYAMN |
| 118 | antibody E2M | WVRQAPGKGLEWVARIRSKYNNYATYYADSVKERF |
| | | TISRDDSKNTAYLQMNNLKTEDTAVYYCVRHRNFGN |
| | | SYLSWFAYWGQGTLVTVSSGGGGSGGGGSGGGG |
| | | SQTVVTQEPSLTVSPGGTVTLTCRSSTGAVTSGYYP |
| | | NWVQQKPGQAPRGLIGATDMRPSGTPARFSGSLLG |
| | | GKAALTLSGVQPEDEAEYYCALWYSNRWVFGGGTK |
| | | LTVL |
| SEQ ID NO: | VH-VL of anti-CD3 | EVQLLESGGGLVQPGGSLKLSCAASGFTFNGYAMN |
| 119 | antibody E2M | WVRQAPGKGLEWVARIRSKYNNYATYYADSVKERF |
| | | TISRDDSKNTAYLQMNNLKTEDTAVYYCVRHRNFGN |
| | | SYLSWFAYWGQGTLVTVSSGGGGSGGGGSGGGG |
| | | SELVVTQEPSLTVSPGGTVTLTCRSSTGAVTSGYYP |
| | | NWVQQKPGQAPRGLIGATDMRPSGTPARFSGSLLG |
| | | GKAALTLSGVQPEDEAEYYCALWYSNRWVFGGGTK |
| | | LTVL |
| SEQ ID NO: | CDR-H1 of anti- | VYAMN |
| 120 | CD3 antibody F70 | |
| SEQ ID NO: | CDR-H2 of anti- | RIRSKYNNYATYYADSVKK |
| 121 | CD3 antibody F70 | |
| SEQ ID NO: | CDR-H3 of anti- | HGNFGNSYISWWAY |
| 122 | CD3 antibody F70 | |
| SEQ ID NO: | VH of anti-CD3 | EVQLVESGGGLVQPGGSLKLSCAASGFTFNVYAMN |
| 123 | antibody F70 | WVRQAPGKGLEWVARIRSKYNNYATYYADSVKKRF |
| | | TISRDDSKNTAYLQMNNLKTEDTAVYYCVRHGNFGN |
| | | SYISWWAYWGQGTLVTVSS |
| SEQ ID NO: | VL of anti-CD3 | QTVVTQEPSLTVSPGGTVTLTCGSSTGAVTSGYYPN |
| 124 | antibody F70 | WVQQKPGQAPRGLIGGTKFLAPGTPARFSGSLLGG |
| | | KAALTLSGVQPEDEAEYYCALWYSNRWVFGGGTKL |
| | | TVL |
| SEQ ID NO: | VH of anti-CD3 | EVQLLESGGGLVQPGGSLKLSCAASGFTFNVYAMN |
| 125 | antibody F70 | WVRQAPGKGLEWVARIRSKYNNYATYYADSVKKRF |
| | | TISRDDSKNTAYLQMNNLKTEDTAVYYCVRHGNFGN |
| | | SYISWWAYWGQGTLVTVSS |
| SEQ ID NO: | VL of anti-CD3 | ELVVTQEPSLTVSPGGTVTLTCGSSTGAVTSGYYPN |
| 126 | antibody F70 | WVQQKPGQAPRGLIGGTKFLAPGTPARFSGSLLGG |
| | | KAALTLSGVQPEDEAEYYCALWYSNRWVFGGGTKL |
| | | TVL |
| SEQ ID NO: | VH-VL of anti-CD3 | EVQLVESGGGLVQPGGSLKLSCAASGFTFNVYAMN |
| 127 | antibody F70 | WVRQAPGKGLEWVARIRSKYNNYATYYADSVKKRF |
| | | TISRDDSKNTAYLQMNNLKTEDTAVYYCVRHGNFGN |
| | | SYISWWAYWGQGTLVTVSSGGGGSGGGGSGGGG |
| | | SQTVVTQEPSLTVSPGGTVTLTCGSSTGAVTSGYYP |
| | | NWVQQKPGQAPRGLIGGTKFLAPGTPARFSGSLLG |
| | | GKAALTLSGVQPEDEAEYYCALWYSNRWVFGGGTK |
| | | LTVL |
| SEQ ID NO: | VH-VL of anti-CD3 | EVQLLESGGGLVQPGGSLKLSCAASGFTFNVYAMN |
| 128 | antibody F70 | WVRQAPGKGLEWVARIRSKYNNYATYYADSVKKRF |
| | | TISRDDSKNTAYLQMNNLKTEDTAVYYCVRHGNFGN |
| | | SYISWWAYWGQGTLVTVSSGGGGSGGGGSGGGG |
| | | SELVVTQEPSLTVSPGGTVTLTCGSSTGAVTSGYYP |
| | | NWVQQKPGQAPRGLIGGTKFLAPGTPARFSGSLLG |
| | | GKAALTLSGVQPEDEAEYYCALWYSNRWVFGGGTK |
| | | LTVL |
| SEQ ID NO: | CDR-L1 of anti- | GSSTGAVTSGNYPN |
| 129 | CD3 antibody | |
| | F12Q | |
| SEQ ID NO: | CDR-L2 of anti- | GTKFLAP |
| 130 | CD3 antibody | |
| | F12Q | |
| SEQ ID NO: | CDR-L3 of anti- | VLWYSNRWV |
| 131 | CD3 antibody | |
| | F12Q | |
| SEQ ID NO: | CDR-H1 of anti- | SYAMN |
| 132 | CD3 antibody | |
| | F12Q | |
| SEQ ID NO: | CDR-H2 of anti- | RIRSKYNNYATYYADSVKG |
| 133 | CD3 antibody | |
| | F12Q | |
| SEQ ID NO: | CDR-H3 of anti- | HGNFGNSYVSWWAY |
| 134 | CD3 antibody | |
| | F12Q | |
| SEQ ID NO: | VH of anti-CD3 | EVQLVESGGGLVQPGGSLKLSCAASGFTFNSYAMN |
| 135 | antibody F12Q | WVRQAPGKGLEWVARIRSKYNNYATYYADSVKGRF |
| | | TISRDDSKNTAYLQMNNLKTEDTAVYYCVRHGNFGN |
| | | SYVSWWAYWGQGTLVTVSS |
| SEQ ID NO: | VL of anti-CD3 | QTVVTQEPSLTVSPGGTVTLTCGSSTGAVTSGNYPN |
| 136 | antibody F12Q | WVQQKPGQAPRGLIGGTKFLAPGTPARFSGSLLGG |
| | | KAALTLSGVQPEDEAEYYCVLWYSNRWVFGGGTKL |
| | | TVL |
| SEQ ID NO: | VH of anti-CD3 | EVQLLESGGGLVQPGGSLKLSCAASGFTFNSYAMN |
| 137 | antibody F12Q | WVRQAPGKGLEWVARIRSKYNNYATYYADSVKGRF |
| | | TISRDDSKNTAYLQMNNLKTEDTAVYYCVRHGNFGN |
| | | SYVSWWAYWGQGTLVTVSS |
| SEQ ID NO: | VL of anti-CD3 | ELVVTQEPSLTVSPGGTVTLTCGSSTGAVTSGNYPN |
| 138 | antibody F12Q | WVQQKPGQAPRGLIGGTKFLAPGTPARFSGSLLGG |
| | | KAALTLSGVQPEDEAEYYCVLWYSNRWVFGGGTKL |
| | | TVL |
| SEQ ID NO: | VH-VL of anti-CD3 | EVQLVESGGGLVQPGGSLKLSCAASGFTFNSYAMN |
| 139 | antibody F12Q | WVRQAPGKGLEWVARIRSKYNNYATYYADSVKGRF |
| | | TISRDDSKNTAYLQMNNLKTEDTAVYYCVRHGNFGN |
| | | SYVSWWAYWGQGTLVTVSSGGGGSGGGGSGGGG |
| | | SQTVVTQEPSLTVSPGGTVTLTCGSSTGAVTSGNYP |
| | | NWVQQKPGQAPRGLIGGTKFLAPGTPARFSGSLLG |
| | | GKAALTLSGVQPEDEAEYYCVLWYSNRWVFGGGTK |
| | | LTVL |
| SEQ ID NO: | VH-VL of anti-CD3 | EVQLLESGGGLVQPGGSLKLSCAASGFTFNSYAMN |
| 140 | antibody F12Q | WVRQAPGKGLEWVARIRSKYNNYATYYADSVKGRF |
| | | TISRDDSKNTAYLQMNNLKTEDTAVYYCVRHGNFGN |
| | | SYVSWWAYWGQGTLVTVSSGGGGSGGGGSGGGG |
| | | SELVVTQEPSLTVSPGGTVTLTCGSSTGAVTSGNYP |
| | | NWVQQKPGQAPRGLIGGTKFLAPGTPARFSGSLLG |
| | | GKAALTLSGVQPEDEAEYYCVLWYSNRWVFGGGTK |
| | | LTVL |
| SEQ ID NO: | CDR-H1 of anti- | KYAMN |
| 141 | CD3 antibody I2C | |
| SEQ ID NO: | CDR-H2 of anti- | RIRSKYNNYATYYADSVKD |
| 142 | CD3 antibody I2C | |
| SEQ ID NO: | CDR-H3 of anti- | HGNFGNSYISYWAY |
| 143 | CD3 antibody I2C | |
| SEQ ID NO: | VH of anti-CD3 | EVQLVESGGGLVQPGGSLKLSCAASGFTFNKYAMN |
| 144 | antibody I2C | WVRQAPGKGLEWVARIRSKYNNYATYYADSVKDRF |
| | | TISRDDSKNTAYLQMNNLKTEDTAVYYCVRHGNFGN |
| | | SYISYWAYWGQGTLVTVSS |
| SEQ ID NO: | VL of anti-CD3 | QTVVTQEPSLTVSPGGTVTLTCGSSTGAVTSGNYPN |
| 145 | antibody I2C | WVQQKPGQAPRGLIGGTKFLAPGTPARFSGSLLGG |
| | | KAALTLSGVQPEDEAEYYCVLWYSNRWVFGGGTKL |
| | | TVL |
| SEQ ID NO: | VH of anti-CD3 | EVQLLESGGGLVQPGGSLKLSCAASGFTFNKYAMN |
| 146 | antibody I2C | WVRQAPGKGLEWVARIRSKYNNYATYYADSVKDRF |
| | | TISRDDSKNTAYLQMNNLKTEDTAVYYCVRHGNFGN |
| | | SYISYWAYWGQGTLVTVSS |
| SEQ ID NO: | VL of anti-CD3 | ELVVTQEPSLTVSPGGTVTLTCGSSTGAVTSGNYPN |
| 147 | antibody I2C | WVQQKPGQAPRGLIGGTKFLAPGTPARFSGSLLGG |
| | | KAALTLSGVQPEDEAEYYCVLWYSNRWVFGGGTKL |
| | | TVL |
| SEQ ID NO: | VH-VL of anti-CD3 | EVQLVESGGGLVQPGGSLKLSCAASGFTFNKYAMN |
| 148 | antibody I2C | WVRQAPGKGLEWVARIRSKYNNYATYYADSVKDRF |
| | | TISRDDSKNTAYLQMNNLKTEDTAVYYCVRHGNFG |
| | | NSYISYWAYWGQGTLVTVSSGGGGSGGGGSGGGGS |
| | | QTVVTQEPSLTVSPGGTVTLTCGSSTGAVTSGNYPN |
| | | WVQQKPGQAPRGLIGGTKFLAPGTPARFSGSLLGG |
| | | KAALTLSGVQPEDEAEYYCVLWYSNRWVFGGGTKL |
| | | TVL |
| SEQ ID NO: | VH-VL of anti-CD3 | EVQLLESGGGLVQPGGSLKLSCAASGFTFNKYAMN |
| 149 | antibody I2C | WVRQAPGKGLEWVARIRSKYNNYATYYADSVKDRF |
| | | TISRDDSKNTAYLQMNNLKTEDTAVYYCVRHGNFG |
| | | NSYISYWAYWGQGTLVTVSSGGGGSGGGGSGGGGS |
| | | ELVVTQEPSLTVSPGGTVTLTCGSSTGAVTSGNYP |
| | | NWVQQKPGQAPRGLIGGTKFLAPGTPARFSGSLLG |
| | | GKAALTLSGVQPEDEAEYYCVLWYSNRWVFGGGTK |
| | | LTVL |
| SEQ ID NO: | Ab85 full length | EVQLVQSGAEVKKPGESLKISCKGSGYSFTNYWIG |
| 150 | heavy chain | WVRQMPGKGLEWMAIINPRDSDTRYRPSFQGQVTI |
| | sequence; | SADKSISTAYLQWSSLKASDTAMYYCARHGRGYEG |
| | constant region | YEGAFDIWGQGTLVTVSSASTKGPSVFPLAPSSKST |
| | underlined | SGGTAALGCLVKDYFPEPVTVSWNSGALTSGVHTF |
| | | PAVLQSSGLYSLSSVVTVPSSSLGTQTYICNVNHKP |
| | | SNTKVDKKVEPKSCDKTHTCPPCPAPELLGGPSVFL |
| | | FPPKPKDTLMISRTPEVTCVVVDVSHEDPEVKFNWY |
| | | VDGVEVHNAKTKPREEQYNSTYRVVSVLTVLHQDWL |
| | | NGKEYKCKVSNKALPAPIEKTISKAKGQPREPQVYT |
| | | LPPSRDELTKNQVSLTCLVKGFYPSDIAVEWESNGQ |
| | | PENNYKTTPPVLDSDGSFFLYSKLTVDKSRWQQGN |
| | | VFSCSVMHEALHNHYTQKSLSLSPGK |
| SEQ ID NO: | Ab85 full length | DIQMTQSPSSLSASVGDRVTITCRSSQGIRSDLGWY |
| 151 | light chain; | QQKPGKAPKLLIYDASNLETGVPSRFSGSGSGTDFT |
| | constant region | LTISSLQPEDFATYYCQQANGFPLTFGGGTKVEIKRT |
| | underlined | VAAPSVFIFPPSDEQLKSGTASVVCLLNNFYPREAKV |
| | | QWKVDNALQSGNSQESVTEQDSKDSTYSLSSTLTL |
| | | SKADYEKHKVYACEVTHQGLSSPVTKSFNRGEC |
| SEQ ID NO: | Ab67 Heavy chain | EVQLVESGGGLVQPGGSLRLSCAASGFTFSDADMD |
| 152 | HC constant | WVRQAPGKGLEWVGRTRNKAGSYTTEYAASVKGR |
| | region underlined | FTISRDDSKNSLYLQMNSLKTEDTAVYYCAREPKYW |
| | | IDFDLWGRGTLVTVSSASTKGPSVFPLAPSSKSTSG |
| | | GTAALGCLVKDYFPEPVTVSWNSGALTSGVHTFPAV |
| | | LQSSGLYSLSSVVTVPSSSLGTQTYICNVNHKPSNT |
| | | KVDKKVEPKSCDKTHTCPPCPAPELLGGPSVFLFPP |
| | | KPKDTLMISRTPEVTCVVVDVSHEDPEVKFNWYVDG |
| | | VEVHNAKTKPREEQYNSTYRVVSVLTVLHQDWLNGK |
| | | EYKCKVSNKALPAPIEKTISKAKGQPREPQVYTLPP |
| | | SRDELTKNQVSLTCLVKGFYPSDIAVEWESNGQPEN |
| | | NYKTTPPVLDSDGSFFLYSKLTVDKSRWQQGNVFS |
| | | CSVMHEALHNHYTQKSLSLSPGK |
| SEQ ID NO: | Ab67 Light chain | DIQMTQSPSSLSASVGDRVTITCRASQSISSYLNWY |
| 153 | LC constant region | QQKPGKAPKLLIYAASSLQSGVPSRFSGSGSGTDFT |
| | underlined | LTISSLQPEDFATYYCQQSYIAPYTFGGGTKVEIKRT |
| | | VAAPSVFIFPPSDEQLKSGTASVVCLLNNFYPREAKV |
| | | QWKVDNALQSGNSQESVTEQDSKDSTYSLSSTLTL |
| | | SKADYEKHKVYACEVTHQGLSSPVTKSFNRGEC |

### OTHER EMBODIMENTS

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

1. A bispecific binding polypeptide comprising
a first antigen binding moiety that binds to CD117 expressed on a hematopoietic stem cell (HSC) or a hematopoietic progenitor cell; and
a second antigen binding moiety that binds to an antigen expressed on a T cell.

2. (canceled)

3. The bispecific binding polypeptide of claim 1, wherein the first antigen binding moiety comprises an anti-CD117 single-chain variable fragment (scFv) and the second antigen binding moiety comprises an anti-CD3 scFv.

4. (canceled)

5. The bispecific binding polypeptide of claim 1, wherein the anti-CD117 binding moiety comprises
(i) a heavy chain variable region comprising a CDR1, a CDR2, and a CDR3 having an amino acid sequence as set forth in SEQ ID NOs: 7, 8, and 9, respectively, and a light chain variable region comprising a CDR1, a CDR2, and a CDR3 having an amino acid sequence as set forth in SEQ ID NOs: 10, 11, and 12, respectively; or
(ii) a heavy chain variable region comprising an amino acid sequence as set forth in SEQ ID NO: 13 and a light chain variable region comprising an amino acid sequence as set forth in SEQ ID NO: 14; or;
(iii) a heavy chain variable region comprising a CDR1, a CDR2, and a CDR3 having an amino acid sequence as set forth in SEQ ID NOs: 21, 22, and 23, respectively, and a light chain variable region comprising a CDR1, a CDR2, and a CDR3 having an amino acid sequence as set forth in SEQ ID NOs: 24, 25, and 26, respectively; or
(iv) a heavy chain variable region comprising an amino acid sequence as set forth in SEQ ID NO: 27 and a light chain variable region comprising an amino acid sequence as set forth in SEQ ID NO: 28.

6. The bispecific binding polypeptide of claim 5, wherein the anti-CD3 binding moiety comprises
(i) a heavy chain variable region comprising an amino acid sequence as set forth in SEQ ID NO: 37 and a light chain variable region comprising an amino acid sequence as set forth in SEQ ID NO: 38; or
(ii) a heavy chain variable region comprising an amino acid sequence as set forth in SEQ ID NO: 41 and a light chain variable region comprising an amino acid sequence as set forth in SEQ ID NO: 45.

7. A bispecific antibody, or a bispecific antigen-binding portion thereof, comprising a CD117 binding region and a CD3 binding region, wherein the CD117 binding region comprises
(i) a heavy chain variable region comprising a CDR1, a CDR2, and a CDR3 having an amino acid sequence as set forth in SEQ ID NOs: 7, 8, and 9, respectively, and a light chain variable region comprising a CDR1, a CDR2, and a CDR3 having an amino acid sequence as set forth in SEQ ID NOs: 10, 11, and 12, respectively; or
(ii) a heavy chain variable region comprising an amino acid sequence as set forth in SEQ ID NO: 13 and a light chain variable region comprising an amino acid sequence as set forth in SEQ ID NO: 14; or;
(iii) a heavy chain variable region comprising a CDR1, a CDR2, and a CDR3 having an amino acid sequence as set forth in SEQ ID NOs: 21, 22, and 23, respectively, and a light chain variable region comprising a CDR1, a CDR2, and a CDR3 having an amino acid sequence as set forth in SEQ ID NOs: 24, 25, and 26, respectively; or
(iv) a heavy chain variable region comprising an amino acid sequence as set forth in SEQ ID NO: 27 and a light chain variable region comprising an amino acid sequence as set forth in SEQ ID NO: 28.

8. The bispecific antibody, or a bispecific antigen-binding portion thereof, of claim 7, wherein the CD3 binding region comprises
(i) an amino acid sequence as set forth in SEQ ID NO: 37 and an amino acid sequence as set forth in SEQ ID NO: 38; or
(ii) a heavy chain variable region comprising an amino acid sequence as set forth in SEQ ID NO: 41 and a light chain variable region comprising an amino acid sequence as set forth in SEQ ID NO: 45.

9. (canceled)

10. (canceled)

11. (canceled)

12. The bispecific antibody, or a bispecific antigen-binding portion thereof, of claim 7, wherein the Fc region comprises an amino acid substitution(s) relative to a wild-type Fc region at position L234, L235, H435, or combinations thereof (EU index).

13. (canceled)

14. (canceled)

15. (canceled)

16. The bispecific antibody, or a bispecific antigen-binding portion thereof, of claim 7, wherein the Fc region comprises a first CH3 region comprising amino acid substitutions at positions T366, L368, and Y407 (EU index), and a second CH3 region comprising an amino acid substitution at position T366 (EU index).

17. The bispecific antibody, or a bispecific antigen-binding portion thereof, of claim 16, wherein the amino acid substitution at position T366 is T366S, T366W or T366Y.

18. The bispecific antibody, or a bispecific antigen-binding portion thereof, of claim 16, wherein the amino acid substitution at position L368 is L368A.

19. The bispecific antibody, or a bispecific antigen-binding portion thereof, of claim 16, wherein the amino acid substitution at position Y407 is Y407V or Y407T.

20. (canceled)

21. A pharmaceutical composition comprising a therapeutically effective amount of the bispecific binding polypeptide, bispecific antibody, or a bispecific antigen-binding portion thereof of claim 1.

22. A method of treating a stem cell disorder in a human patient, the method comprising administering to the patient a therapeutically effective amount of the bispecific binding polypeptide, bispecific antibody, or a bispecific antigen-binding portion thereof of claim 1.

23. A method of treating an immunodeficiency disorder in a human patient, the method comprising administering to the patient a therapeutically effective amount of the bispecific binding polypeptide, bispecific antibody, or a bispecific antigen-binding portion thereof of claim 1.

24. The method of claim 23, wherein the immunodeficiency disorder is a congenital immunodeficiency or an acquired immunodeficiency.

25. A method of treating a metabolic disorder in a human patient, the method comprising administering to the patient a therapeutically effective amount of the bispecific binding polypeptide, bispecific antibody, or a bispecific antigen-binding portion thereof of claim 1.

26. The method of claim 25, wherein the metabolic disorder is selected from the group consisting of glycogen storage diseases, mucopolysaccharidoses, Gaucher's Disease, Hurlers Disease, sphingolipidoses, and metachromatic leukodystrophy.

27. A method of treating an autoimmune disorder in a human patient, the method comprising administering to the patient a therapeutically effective amount of the bispecific binding polypeptide, bispecific antibody, or a bispecific antigen-binding portion thereof of claim 1.

28. The method of claim 27, wherein the autoimmune disorder is selected from the group consisting of multiple sclerosis, human systemic lupus, rheumatoid arthritis, inflammatory bowel disease, treating psoriasis, Type 1 diabetes mellitus, acute disseminated encephalomyelitis, Addison's disease, alopecia universalis, ankylosing spondylitisis, antiphospholipid antibody syndrome, aplastic anemia, autoimmune hemolytic anemia, autoimmune hepatitis, autoimmune inner ear disease, autoimmune lymphoproliferative syndrome, autoimmune oophoritis, Balo disease, Behcet's disease, bullous pemphigoid, cardiomyopathy, Chagas' disease, chronic fatigue immune dysfunction syndrome, chronic inflammatory demyelinating polyneuropathy, Crohn's disease, cicatrical pemphigoid, coeliac sprue-dermatitis herpetiformis, cold agglutinin disease, CREST syndrome, Degos disease, discoid lupus, dysautonomia, endometriosis, essential mixed cryoglobulinemia, fibromyalgia-fibromyositis, Goodpasture's syndrome, Grave's disease, Guillain-Barre syndrome, Hashimoto's thyroiditis, Hidradenitis suppurativa, idiopathic and/or acute thrombocytopenic purpura, idiopathic pulmonary fibrosis, IgA neuropathy, interstitial cystitis, juvenile arthritis, Kawasaki's disease, lichen planus, Lyme disease, Meniere disease, mixed connective tissue disease, myasthenia gravis, neuromyotonia, opsoclonus myoclonus syndrome, optic neuritis, Ord's thyroiditis, pemphigus vulgaris, pernicious anemia, polychondritis, polymyositis and dermatomyositis, primary biliary cirrhosis, polyarteritis nodosa, polyglandular syndromes, polymyalgia rheumatica, primary agammaglobulinemia, Raynaud phenomenon, Reiter's syndrome, rheumatic fever, sarcoidosis, scleroderma, Sjögren's syndrome, stiff person syndrome, Takayasu's arteritis, temporal arteritis, ulcerative colitis, uveitis, vasculitis, vitiligo, vulvodynia, and Wegener's granulomatosis.

29. A method of treating a cancer in a human patient, the method comprising administering to the patient a therapeutically effective amount of the bispecific binding polypeptide, bispecific antibody, or a bispecific antigen-binding portion thereof ef-any-ene of claim 1.

30. The method of claim 29, wherein the cancer is selected from the group consisting of leukemia, lymphoma, multiple myeloma, and neuroblastoma.

31. A method of depleting a population of stem cells in a human patient, the method comprising administering to the patient an effective amount of the bispecific binding polypeptide, bispecific antibody, or a bispecific antigen-binding portion thereof of claim 1.

32. The method of claim 31, further comprising administering to the patient a transplant comprising hematopoietic stem cells.

33. A method of selectively depleting hematopoietic stem cells (HSCs) in a human patient in need thereof, said method comprising administering a bispecific antibody, or a bispecific antigen-binding portion thereof, to the human subject in need thereof, such that HSCs are depleted, wherein the bispecific antibody, or a bispecific antigen-binding portion thereof, comprises a first binding moiety that specifically binds to a human HSC cell surface antigen and comprise a second binding moiety that specifically binds to a human T cell surface antigen, wherein the bispecific antibody, or the bispecific antigen binding fragment thereof, comprises an Fc region comprising a first CH3 region of a first heavy chain, and comprises a second CH3 region of a second heavy chain, wherein the first and the second CH3 regions are capable of stable association via a knob-in-hole interaction.

34. The method of claim 33, wherein the first antigen binding moiety binds to a human HSC cell surface antigen selected from the group consisting of CD7, CDwl2, CD13, CD15, CD19, CD21, CD22, CD29, CD30, CD33, CD34, CD36, CD38, CD40, CD41, CD42a, CD42b, CD42c, CD42d, CD43, CD48, CD49b, CD49d, CD49e, CD49f, CD50, CD53, CD55, CD64a, CD68, CD71, CD72, CD73, CD81, CD82, CD85A, CD85K, CD90, CD99, CD104, CD105, CD109, CD110, CD111, CD112, CD114, CD115, CD117, CD123, CD124, CD126, CD127, CD130, CD131, CD133, CD135, CD138, CD151, CD157, CD162, CD164, CD168, CD172a, CD173, CD174, CD175, CD175s, CD176, CD183, CD191, CD200, CD201, CD205, CD217, CD220, CD221, CD222, CD223, CD224, CD225, CD226, CD227, CD228, CD229, CD230, CD235a, CD235b, CD236, CD236R, CD238, CD240, CD242, CD243, CD277, CD292, CDw293, CD295, CD298, CD309, CD318, CD324, CD325, CD338, CD344, CD349 and CD350.

35. (canceled)

36. (canceled)

37. The method of claim 33, wherein the first antigen binding moiety binds to CD117, and wherein the first antigen binding moiety comprises
(i) a heavy chain variable region comprising a CDR1, a CDR2, and a CDR3 having an amino acid sequence as set forth in SEQ ID NOs: 7, 8, and 9, respectively, and comprises a light chain variable region comprising a CDR1, a CDR2, and a CDR3 having an amino acid sequence as set forth in SEQ ID NOs: 10, 11, and 12, respectively; or
(ii) a heavy chain variable region comprising an amino acid sequence as set forth in SEQ ID NO: 13 and a light chain variable region comprising an amino acid sequence as set forth in SEQ ID NO: 14; or;
(iii) a heavy chain variable region comprising a CDR1, a CDR2, and a CDR3 having an amino acid sequence as set forth in SEQ ID NOs: 21, 22, and 23, respectively, and comprises a light chain variable region comprising a CDR1, a CDR2, and a CDR3 having an amino acid sequence as set forth in SEQ ID NOs: 24, 25, and 26, respectively; or
(iv) a heavy chain variable region comprising an amino acid sequence as set forth in SEQ ID NO: 27 and
a light chain variable region comprising an amino acid sequence as set forth in SEQ ID NO: 28.

38. The method of claim 47-37, wherein the second antigen binding moiety binds to CD3, and wherein the second antigen binding moiety comprises
(i) a heavy chain variable region comprising an amino acid sequence as set forth in SEQ ID NO: 37 and
a light chain variable region comprising an amino acid sequence as set forth in SEQ ID NO: 38; or
(ii) a heavy chain variable region comprising an amino acid sequence as set forth in SEQ ID NO: 41 and a light chain variable region comprising an amino acid sequence as set forth in SEQ ID NO: 45.

39. (canceled)

40. (canceled)

41. (canceled)

42. The method of claim 33, wherein the first CH3 region comprises amino acid substitutions at positions T366, L368, and Y407 (EU index), and the second CH3 region comprises an amino acid substitution at position T366 (EU index).

43. The method of claim 42, wherein the amino acid substitution at position T366 is T366S, T366W or T366Y.

44. The method of claim 42, wherein the amino acid substitution at position L368 is L368A.

45. The method of claim 42, wherein the amino acid substitution at position Y407 is Y407V or Y407T.

46. (canceled)

47. The method of claim 33, wherein the patient has a stem cell disorder and is in need of a transplant.

48. The method of claim 47, further comprising administering an HSC transplant to the patient following depletion.

49. The method of claim 33, wherein the patient has an immunodeficiency disorder, a metabolic disorder, an autoimmune disorder, or cancer.


SEQUENCE LISTING (153 sequences)

SEQ ID NO: 1 - protein, 5 aa, Artificial Sequence
  Description of Artificial Sequence Synthetic peptide
SYWIG

SEQ ID NO: 2 - protein, 17 aa, Artificial Sequence
  Description of Artificial Sequence Synthetic peptide
IIYPGDSDTRYSPSFQG

SEQ ID NO: 3 - protein, 14 aa, Artificial Sequence
  Description of Artificial Sequence Synthetic peptide
HGRGYNGYEGAFDI

SEQ ID NO: 4 - protein, 11 aa, Artificial Sequence
  Description of Artificial Sequence Synthetic peptide
RASQGISSALA

SEQ ID NO: 5 - protein, 7 aa, Artificial Sequence
  Description of Artificial Sequence Synthetic peptide
DASSLES

SEQ ID NO: 6 - protein, 10 aa, Artificial Sequence
  Description of Artificial Sequence Synthetic peptide
CQQFNSYPLT

SEQ ID NO: 7 - protein, 5 aa, Artificial Sequence
  Description of Artificial Sequence Synthetic peptide
NYWIG

SEQ ID NO: 8 - protein, 17 aa, Artificial Sequence
  Description of Artificial Sequence Synthetic peptide
IINPRDSDTRYRPSFQG

SEQ ID NO: 9 - protein, 14 aa, Artificial Sequence
  Description of Artificial Sequence Synthetic peptide
HGRGYEGYEGAFDI

SEQ ID NO: 10 - protein, 11 aa, Artificial Sequence
  Description of Artificial Sequence Synthetic peptide
RSSQGIRSDLG

SEQ ID NO: 11 - protein, 7 aa, Artificial Sequence
  Description of Artificial Sequence Synthetic peptide
DASNLET

SEQ ID NO: 12 - protein, 9 aa, Artificial Sequence
  Description of Artificial Sequence Synthetic peptide
QQANGFPLT

SEQ ID NO: 13 - protein, 123 aa, Artificial Sequence
  Description of Artificial Sequence Synthetic polypeptide
EVQLVQSGAEVKKPGESLKISCKGSGYSFTNYWIGWVRQMPGKGLEWMAIINPRDSDTRYRPSFQGQVTISADKSISTAYLQWSSLKASDTAMYYCARHGRGYEGYEGAFDIWGQGTLVTVSS

SEQ ID NO: 14 - protein, 107 aa, Artificial Sequence
  Description of Artificial Sequence Synthetic polypeptide
DIQMTQSPSSLSASVGDRVTITCRSSQGIRSDLGWYQQKPGKAPKLLIYDASNLETGVPSRFSGSGSGTDFTLTISSLQPEDFATYYCQQANGFPLTFGGGTKVEIK

SEQ ID NO: 15 - protein, 330 aa, Artificial Sequence [sequence omitted]

SEQ ID NO: 16 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 19 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 21 - protein, 9 aa, Artificial Sequence
  Description of Artificial Sequence Synthetic peptide
FTFSDADMD

SEQ ID NO: 22 - protein, 19 aa, Artificial Sequence
  Description of Artificial Sequence Synthetic peptide
RTRNKAGSYTTEYAASVKG

SEQ ID NO: 23 - protein, 12 aa, Artificial Sequence
  Description of Artificial Sequence Synthetic peptide
AREPKYWIDFDL

SEQ ID NO: 24 - protein, 11 aa, Artificial Sequence
  Description of Artificial Sequence Synthetic peptide
RASQSISSYLN

SEQ ID NO: 25 - protein, 7 aa, Artificial Sequence
  Description of Artificial Sequence Synthetic peptide
AASSLQS

SEQ ID NO: 26 - protein, 9 aa, Artificial Sequence
  Description of Artificial Sequence Synthetic peptide
QQSYIAPYT

SEQ ID NO: 27 - protein, 121 aa, Artificial Sequence
  Description of Artificial Sequence Synthetic polypeptide
EVQLVESGGGLVQPGGSLRLSCAASGFTFSDADMDWVRQAPGKGLEWVGRTRNKAGSYTTEYAASVKGRFTISRDDSKNSLYLQMNSLKTEDTAVYYCAREPKYWIDFDLWGRGTLVTVSS

SEQ ID NO: 28 - protein, 107 aa, Artificial Sequence
  Description of Artificial Sequence Synthetic polypeptide
DIQMTQSPSSLSASVGDRVTITCRASQSISSYLNWYQQKPGKAPKLLIYAASSLQSGVPSRFSGSGSGTDFTLTISSLQPEDFATYYCQQSYIAPYTFGGGTKVEIK

SEQ ID NO: 29 - protein, 123 aa, Artificial Sequence
  Description of Artificial Sequence Synthetic polypeptide
QVQLVQSGAAVKKPGESLKISCKGSGYRFTSYWIGWVRQMPGKGLEWMGIIYPGDSDTRYSPSFQGQVTISAGKSISTAYLQWSSLKASDTAMYYCARHGRGYNGYEGAFDIWGQGTMVTVSS

SEQ ID NO: 30 - protein, 107 aa, Artificial Sequence [sequence omitted]

SEQ ID NO: 31 - DNA, 1311 bp, Homo sapiens
agtctagctgctgcacaggctggctggctggctggctgctaagggctgctccacgcttttgccggaggacagagactgacatggaacaggggaagggcctggctgtcctcatcctggctatcattcttcttcaaggtactttggcccagtcaatcaaaggaaaccacttggttaaggtgtatgactatcaagaagatggttcggtacttctgacttgtgatgcagaagccaaaaatatcacatggtttaaagatgggaagatgatcggcttcctaactgaagataaaaaaaaatggaatctgggaagtaatgccaaggaccctcgagggatgtatcagtgtaaaggatcacagaacaagtcaaaaccactccaagtgtattacagaatgtgtcagaactgcattgaactaaatgcagccaccatatctggctttctctttgctgaaatcgtcagcattttcgtccttgctgttggggtctacttcattgctggacaggatggagttcgccagtcgagagcttcagacaagcagactctgttgcccaatgaccagctctaccagcccctcaaggatcgagaagatgaccagtacagccaccttcaaggaaaccagttgaggaggaattgaactcaggactcagagtagtccaggtgttctcctcctattcagttcccagaatcaaagcaatgcattttggaaagctcctagcagagagactttcagccctaaatctagactcaaggttcccagagatgacaaatggagaagaaaggccatcagagcaaatttgggggtttctcaaataaaataaaaataaaaacaaatactgtgtttcagaagcgccacctattggggaaaattgtaaaagaaaaatgaaaagatcaaataaccccctggatttgaatataattttttgtgttgtaatttttatttcgtttttgtataggttataattcacatggctcaaatattcagtgaaagctctccctccaccgccatcccctgctacccagtgaccctgttgccctcttcagagacaaattagtttctcttttttttttttttttttttttttttgagacagtctggctctgtcacccaggctgaaatgcagtggcaccatctcggctcactgcaacctctgcctcctgggttcaagcgattctcctgcctcagcctcccgggcagctgggattacaggcacacactaccacacctggctaatttttgtatttttagtagagacagggttttgctctgttggccaagctggtctcgaactcctgacctcaagtgatccgcccgcctc

SEQ ID NO: 32 - protein, 182 aa, Homo sapiens
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 33 - DNA, 771 bp, Homo sapiens
agagaagcagacatcttctagttcctcccccactctcctctttccggtacctgtgagtcagctaggggagggcagctctcacccaggctgatagttcggtgacctggctttatctactggatgagttccgctgggagatggaacatagcacgtttctctctggcctggtactggctacccttctctcgcaagtgagccccttcaagatacctatagaggaacttgaggacagagtgtttgtgaattgcaataccagcatcacatgggtagagggaacggtgggaacactgctctcagacattacaagactggacctgggaaaacgcatcctggacccacgaggaatatataggtgtaatgggacagatatatacaaggacaaagaatctaccgtgcaagttcattatcgaatgtgccagagctgtgtggagctggatccagccaccgtggctggcatcattgtcactgatgtcattgccactctgctccttgctttgggagtcttctgctttgctggacatgagactggaaggctgtctggggctgccgacacacaagctctgttgaggaatgaccaggtctatcagcccctccgagatcgagatgatgctcagtacagccaccttggaggaaactgggctcggaacaagtgaacctgagactggtggcttctagaagcagccattaccaactgtaccttcccttcttgctcagccaataaatatatcctctttcactcagaaaaaaaaaaaaaaaaaaaaaaaaaaaaaa

SEQ ID NO: 34 - protein, 171 aa, Homo sapiens
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 35 - DNA, 1534 bp, Homo sapiens
tattgtcagagtcctcttgtttggccttctaggaaggctgtgggacccagctttcttcaaccagtccaggtggaggcctctgccttgaacgtttccaagtgaggtaaaacccgcaggcccagaggcctctctacttcctgtgtggggttcagaaaccctcctcccctcccagcctcaggtgcctgcttcagaaaatgaagtagtaagtctgctggcctccgccatcttagtaaagtaacagtcccatgaaacaaagatgcagtcgggcactcactggagagttctgggcctctgcctcttatcagttggcgtttgggggcaagatggtaatgaagaaatgggtggtattacacagacaccatataaagtctccatctctggaaccacagtaatattgacatgccctcagtatcctggatctgaaatactatggcaacacaatgataaaaacataggcggtgatgaggatgataaaaacataggcagtgatgaggatcacctgtcactgaaggaattttcagaattggagcaaagtggttattatgtctgctaccccagaggaagcaaaccagaagatgcgaacttttatctctacctgagggcaagagtgtgtgagaactgcatggagatggatgtgatgtcggtggccacaattgtcatagtggacatctgcatcactgggggcttgctgctgctggtttactactggagcaagaatagaaaggccaaggccaagcctgtgacacgaggagcgggtgctggcggcaggcaaaggggacaaaacaaggagaggccaccacctgttcccaacccagactatgagcccatccggaaaggccagcgggacctgtattctggcctgaatcagagacgcatctgaccctctggagaacactgcctcccgctggcccaggtctcctctccagtccccctgcgactccctgtttcctgggctagtcttggaccccacgagagagaatcgttcctcagcctcatggtgaactcgcgccctccagcctgatcccccgctccctcctccctgccttctctgctggtacccagtcctaaaatattgctgcttcctcttcctttgaagcatcatcagtagtcacaccctcacagctggcctgccctcttgccaggatatttatttgtgctattcactcccttccctttggatgtaacttctccgttcagttccctccttttcttgcatgtaagttgtcccccatcccaaagtattccatctacttttctatcgccgtccccttttgcagccctctctggggatggactgggtaaatgttgacagaggccctgccccgttcacagatcctggccctgagccagccctgtgctcctccctcccccaacactccctaccaaccccctaatcccctactccctccaccccccctccactgtaggccactggatggtcatttgcatctccgtaaatgtgctctgctcctcagctgagagagaaaaaaataaactgtatttggctgcaagaaaaaaaaaaaaaaaaaaaaaa

SEQ ID NO: 36 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 37 - protein, 125 aa, Artificial Sequence
  Description of Artificial Sequence Synthetic polypeptide
EVQLVESGGGLVQPGGSLRLSCAASGFTFNSYAMNWVRQAPGKGLEWVARIRSKYNNYATYYADSVKGRFTISRDDSKNTAYLQMNSLKTEDTAVYYCVRHGNFGNSYVSWWAYWGQGTLVTVSS

SEQ ID NO: 38 - protein, 109 aa, Artificial Sequence
  Description of Artificial Sequence Synthetic polypeptide
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 39 - protein, 446 aa, Artificial Sequence
  Description of Artificial Sequence Synthetic polypeptide
EVQLVESGGGLVQPGKSLKLSCEASGFTFSGYGMHWVRQAPGRGLESVAYITSSSINIKYADAVKGRFTVSRDNAKNLLFLQMNILKSEDTAMYYCARFDWDKNYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 40 - protein, 214 aa, Artificial Sequence
  Description of Artificial Sequence Synthetic polypeptide
DIQMTQSPSSLPASLGDRVTINCQASQDISNYLNWYQQKPGKAPKLLIYYTNKLADGVPSRFSGSGSGRDSSFTISSLESEDIGSYYCQQYYNYPWTFGPGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 119 aa, Artificial Sequence
  Description of Artificial Sequence Synthetic polypeptide
EVQLVQSGAEVKKPGASVKVSCKASGYTFTNYYIHWVRQAPGQGLEWIGWIYPGDGNTKYNEKFKGRATLTADTSTSTAYLELSSLRSEDTAVYYCARDSYSNYYFDYWGQGTLVTVSS

SEQ ID NO: 42 - protein, 8 aa, Artificial Sequence
  Description of Artificial Sequence Synthetic peptide
GYTFTNYY

SEQ ID NO: 43 - protein, 8 aa, Artificial Sequence
  Description of Artificial Sequence Synthetic peptide
IYPGDGNT

SEQ ID NO: 44 - protein, 12 aa, Artificial Sequence
  Description of Artificial Sequence Synthetic peptide
ARDSYSNYYFDY

SEQ ID NO: 45 - protein, 112 aa, Artificial Sequence
  Description of Artificial Sequence Synthetic polypeptide
DIVMTQSPDSLAVSLGERATINCKSSQSLLNSRTRKNYLAWYQQKPGQPPKLLIYWASTRESGVPDRFSGSGSGTDFTLTISSLQAEDVAVYYCTQSFILRTFGQGTKVEIK

SEQ ID NO: 46 - protein, 12 aa, Artificial Sequence
  Description of Artificial Sequence Synthetic peptide
QSLLNSRTRKNY

SEQ ID NO: 47 - protein, 3 aa, Artificial Sequence
  Description of Artificial Sequence Synthetic peptide
WAS

SEQ ID NO: 48 - protein, 8 aa, Artificial Sequence
  Description of Artificial Sequence Synthetic peptide
TQSFILRT

SEQ ID NO: 49 - protein, 449 aa, Artificial Sequence
  Description of Artificial Sequence Synthetic polypeptide
EVQLVQSGAEVKKPGASVKVSCKASGYTFTNYYIHWVRQAPGQGLEWIGWIYPGDGNTKYNEKFKGRATLTADTSTSTAYLELSSLRSEDTAVYYCARDSYSNYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 50 - protein, 219 aa, Artificial Sequence
  Description of Artificial Sequence Synthetic polypeptide
DIVMTQSPDSLAVSLGERATINCKSSQSLLNSRTRKNYLAWYQQKPGQPPKLLIYWASTRESGVPDRFSGSGSGTDFTLTISSLQAEDVAVYYCTQSFILRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 5 aa, Artificial Sequence
  Description of Artificial Sequence Synthetic peptide
IYAMN

SEQ ID NO: 52 - protein, 19 aa, Artificial Sequence
  Description of Artificial Sequence Synthetic peptide
RIRSKYNNYATYYADSVKS

SEQ ID NO: 53 - protein, 14 aa, Artificial Sequence
  Description of Artificial Sequence Synthetic peptide
HGNFGNSYVSFFAY

SEQ ID NO: 54 - protein, 125 aa, Artificial Sequence
  Description of Artificial Sequence Synthetic polypeptide
EVQLVESGGGLVQPGGSLKLSCAASGFTFNIYAMNWVRQAPGKGLEWVARIRSKYNNYATYYADSVKSRFTISRDDSKNTAYLQMNNLKTEDTAVYYCVRHGNFGNSYVSFFAYWGQGTLVTVSS

SEQ ID NO: 55 - protein, 109 aa, Artificial Sequence
  Description of Artificial Sequence Synthetic polypeptide
QTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 56 - protein, 125 aa, Artificial Sequence
  Description of Artificial Sequence Synthetic polypeptide
EVQLLESGGGLVQPGGSLKLSCAASGFTFNIYAMNWVRQAPGKGLEWVARIRSKYNNYATYYADSVKSRFTISRDDSKNTAYLQMNNLKTEDTAVYYCVRHGNFGNSYVSFFAYWGQGTLVTVSS

SEQ ID NO: 57 - protein, 109 aa, Artificial Sequence
  Description of Artificial Sequence Synthetic polypeptide
ELVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 58 - protein, 249 aa, Artificial Sequence
  Description of Artificial Sequence Synthetic polypeptide
EVQLVESGGGLVQPGGSLKLSCAASGFTFNIYAMNWVRQAPGKGLEWVARIRSKYNNYATYYADSVKSRFTISRDDSKNTAYLQMNNLKTEDTAVYYCVRHGNFGNSYVSFFAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 59 - protein, 249 aa, Artificial Sequence
  Description of Artificial Sequence Synthetic polypeptide
EVQLLESGGGLVQPGGSLKLSCAASGFTFNIYAMNWVRQAPGKGLEWVARIRSKYNNYATYYADSVKSRFTISRDDSKNTAYLQMNNLKTEDTAVYYCVRHGNFGNSYVSFFAYWGQGTLVTVSSGGGGSGGGGSGGGGSELVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 60 - protein, 14 aa, Artificial Sequence
  Description of Artificial Sequence Synthetic peptide
GSSTGAVTSGYYPN

SEQ ID NO: 61 - protein, 7 aa, Artificial Sequence
  Description of Artificial Sequence Synthetic peptide
GTKFLAP

SEQ ID NO: 62 - protein, 9 aa, Artificial Sequence
  Description of Artificial Sequence Synthetic peptide
ALWYSNRWV

SEQ ID NO: 63 - protein, 5 aa, Artificial Sequence
  Description of Artificial Sequence Synthetic peptide
KYAMN

SEQ ID NO: 64 - protein, 19 aa, Artificial Sequence
  Description of Artificial Sequence Synthetic peptide
RIRSKYNNYATYYADSVKD

SEQ ID NO: 65 - protein, 14 aa, Artificial Sequence
  Description of Artificial Sequence Synthetic peptide
HGNFGNSYISYWAY

SEQ ID NO: 66 - protein, 125 aa, Artificial Sequence
  Description of Artificial Sequence Synthetic polypeptide
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 67 - protein, 109 aa, Artificial Sequence; identical to SEQ ID NO: 55
  Description of Artificial Sequence Synthetic polypeptide

SEQ ID NO: 68 - protein, 125 aa, Artificial Sequence
  Description of Artificial Sequence Synthetic polypeptide
EVQLLESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 69 - protein, 109 aa, Artificial Sequence; identical to SEQ ID NO: 57
  Description of Artificial Sequence Synthetic polypeptide

SEQ ID NO: 70 - protein, 249 aa, Artificial Sequence
  Description of Artificial Sequence Synthetic polypeptide
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 71 - protein, 249 aa, Artificial Sequence
  Description of Artificial Sequence Synthetic polypeptide
EVQLLESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSELVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 72 - protein, 5 aa, Artificial Sequence
  Description of Artificial Sequence Synthetic peptide
SYAMN

SEQ ID NO: 73 - protein, 19 aa, Artificial Sequence
  Description of Artificial Sequence Synthetic peptide
RIRSKYNNYATYYADSVKG

SEQ ID NO: 74 - protein, 14 aa, Artificial Sequence
  Description of Artificial Sequence Synthetic peptide
HGNFGNSYLSFWAY

SEQ ID NO: 75 - protein, 125 aa, Artificial Sequence
  Description of Artificial Sequence Synthetic polypeptide
EVQLVESGGGLEQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYLSFWAYWGQGTLVTVSS

SEQ ID NO: 76 - protein, 109 aa, Artificial Sequence; identical to SEQ ID NO: 55
  Description of Artificial Sequence Synthetic polypeptide

SEQ ID NO: 77 - protein, 125 aa, Artificial Sequence
  Description of Artificial Sequence Synthetic polypeptide
EVQLLESGGGLEQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYLSFWAYWGQGTLVTVSS

SEQ ID NO: 78 - protein, 109 aa, Artificial Sequence; identical to SEQ ID NO: 57
  Description of Artificial Sequence Synthetic polypeptide

SEQ ID NO: 79 - protein, 249 aa, Artificial Sequence
  Description of Artificial Sequence Synthetic polypeptide
EVQLVESGGGLEQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYLSFWAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 80 - protein, 249 aa, Artificial Sequence
  Description of Artificial Sequence Synthetic polypeptide
EVQLLESGGGLEQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYLSFWAYWGQGTLVTVSSGGGGSGGGGSGGGGSELVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 81 - protein, 5 aa, Artificial Sequence
  Description of Artificial Sequence Synthetic peptide
RYAMN

SEQ ID NO: 82 - protein, 19 aa, Artificial Sequence
  Description of Artificial Sequence Synthetic peptide
RIRSKYNNYATYYADSVKG

SEQ ID NO: 83 - protein, 14 aa, Artificial Sequence
  Description of Artificial Sequence Synthetic peptide
HGNFGNSYLSYFAY

SEQ ID NO: 84 - protein, 125 aa, Artificial Sequence
  Description of Artificial Sequence Synthetic polypeptide
EVQLVESGGGLVQPGGSLKLSCAASGFTFNRYAMNWVRQAPGKGLEWVARIRSKYNNYATYYADSVKGRFTISRDDSKNTAYLQMNNLKTEDTAVYYCVRHGNFGNSYLSYFAYWGQGTLVTVSS

SEQ ID NO: 85 - protein, 109 aa, Artificial Sequence; identical to SEQ ID NO: 55
  Description of Artificial Sequence Synthetic polypeptide

SEQ ID NO: 86 - protein, 125 aa, Artificial Sequence
  Description of Artificial Sequence Synthetic polypeptide
EVQLLESGGGLVQPGGSLKLSCAASGFTFNRYAMNWVRQAPGKGLEWVARIRSKYNNYATYYADSVKGRFTISRDDSKNTAYLQMNNLKTEDTAVYYCVRHGNFGNSYLSYFAYWGQGTLVTVSS

SEQ ID NO: 87 - protein, 109 aa, Artificial Sequence; identical to SEQ ID NO: 57
  Description of Artificial Sequence Synthetic polypeptide

SEQ ID NO: 88 - protein, 249 aa, Artificial Sequence
  Description of Artificial Sequence Synthetic polypeptide
EVQLVESGGGLVQPGGSLKLSCAASGFTFNRYAMNWVRQAPGKGLEWVARIRSKYNNYATYYADSVKGRFTISRDDSKNTAYLQMNNLKTEDTAVYYCVRHGNFGNSYLSYFAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 89 - protein, 249 aa, Artificial Sequence
  Description of Artificial Sequence Synthetic polypeptide
EVQLLESGGGLVQPGGSLKLSCAASGFTFNRYAMNWVRQAPGKGLEWVARIRSKYNNYATYYADSVKGRFTISRDDSKNTAYLQMNNLKTEDTAVYYCVRHGNFGNSYLSYFAYWGQGTLVTVSSGGGGSGGGGSGGGGSELVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 90 - protein, 5 aa, Artificial Sequence
  Description of Artificial Sequence Synthetic peptide
VYAMN

SEQ ID NO: 91 - protein, 19 aa, Artificial Sequence
  Description of Artificial Sequence Synthetic peptide
RIRSKYNNYATYYADSVKK

SEQ ID NO: 92 - protein, 14 aa, Artificial Sequence
  Description of Artificial Sequence Synthetic peptide
HGNFGNSYLSWWAY

SEQ ID NO: 93 - protein, 125 aa, Artificial Sequence
  Description of Artificial Sequence Synthetic polypeptide
EVQLVESGGGLVQPGGSLKLSCAASGFTFNVYAMNWVRQAPGKGLEWVARIRSKYNNYATYYADSVKKRFTISRDDSKNTAYLQMNNLKTEDTAVYYCVRHGNFGNSYLSWWAYWGQGTLVTVSS

SEQ ID NO: 94 - protein, 109 aa, Artificial Sequence
  Description of Artificial Sequence Synthetic polypeptide
QTVVTQEPSLTVSPGGTVTLTCRSSTGAVTSGYYPNWVQQKPGQAPRGLIGATDMRPSGTPARFSGSLLGGKAALTLSGVQPEDEAEYYCALWYSNRWVFGGGTKLTVL

SEQ ID NO: 95 - protein, 125 aa, Artificial Sequence
  Description of Artificial Sequence Synthetic polypeptide
EVQLLESGGGLVQPGGSLKLSCAASGFTFNVYAMNWVRQAPGKGLEWVARIRSKYNNYATYYADSVKKRFTISRDDSKNTAYLQMNNLKTEDTAVYYCVRHGNFGNSYLSWWAYWGQGTLVTVSS

SEQ ID NO: 96 - protein, 109 aa, Artificial Sequence
  Description of Artificial Sequence Synthetic polypeptide
ELVVTQEPSLTVSPGGTVTLTCRSSTGAVTSGYYPNWVQQKPGQAPRGLIGATDMRPSGTPARFSGSLLGGKAALTLSGVQPEDEAEYYCALWYSNRWVFGGGTKLTVL

SEQ ID NO: 97 - protein, 249 aa, Artificial Sequence
  Description of Artificial Sequence Synthetic polypeptide
EVQLVESGGGLVQPGGSLKLSCAASGFTFNVYAMNWVRQAPGKGLEWVARIRSKYNNYATYYADSVKKRFTISRDDSKNTAYLQMNNLKTEDTAVYYCVRHGNFGNSYLSWWAYWGQGTLVTVSSGGGGSGGGGSGGGGSQTVVTQEPSLTVSPGGTVTLTCRSSTGAVTSGYYPNWVQQKPGQAPRGLIGATDMRPSGTPARFSGSLLGGKAALTLSGVQPEDEAEYYCALWYSNRWVFGGGTKLTVL

SEQ ID NO: 98 - protein, 249 aa, Artificial Sequence
  Description of Artificial Sequence Synthetic polypeptide
EVQLLESGGGLVQPGGSLKLSCAASGFTFNVYAMNWVRQAPGKGLEWVARIRSKYNNYATYYADSVKKRFTISRDDSKNTAYLQMNNLKTEDTAVYYCVRHGNFGNSYLSWWAYWGQGTLVTVSSGGGGSGGGGSGGGGSELVVTQEPSLTVSPGGTVTLTCRSSTGAVTSGYYPNWVQQKPGQAPRGLIGATDMRPSGTPARFSGSLLGGKAALTLSGVQPEDEAEYYCALWYSNRWVFGGGTKLTVL

SEQ ID NO: 99 - protein, 5 aa, Artificial Sequence
  Description of Artificial Sequence Synthetic peptide
KYAMN

SEQ ID NO: 100 - protein, 19 aa, Artificial Sequence
  Description of Artificial Sequence Synthetic peptide
RIRSKYNNYATYYADSVKS

SEQ ID NO: 101 - protein, 14 aa, Artificial Sequence
  Description of Artificial Sequence Synthetic peptide
HGNFGNSYTSYYAY

SEQ ID NO: 102 - protein, 125 aa, Artificial Sequence
  Description of Artificial Sequence Synthetic polypeptide
EVQLVESGGGLVQPGGSLKLSCAASGFTFNKYAMNWVRQAPGKGLEWVARIRSKYNNYATYYADSVKSRFTISRDDSKNTAYLQMNNLKTEDTAVYYCVRHGNFGNSYTSYYAYWGQGTLVTVSS

SEQ ID NO: 103 - protein, 109 aa, Artificial Sequence; identical to SEQ ID NO: 55
  Description of Artificial Sequence Synthetic polypeptide

SEQ ID NO: 104 - protein, 125 aa, Artificial Sequence
  Description of Artificial Sequence Synthetic polypeptide
EVQLLESGGGLVQPGGSLKLSCAASGFTFNKYAMNWVRQAPGKGLEWVARIRSKYNNYATYYADSVKSRFTISRDDSKNTAYLQMNNLKTEDTAVYYCVRHGNFGNSYTSYYAYWGQGTLVTVSS

SEQ ID NO: 105 - protein, 109 aa, Artificial Sequence; identical to SEQ ID NO: 57
  Description of Artificial Sequence Synthetic polypeptide

SEQ ID NO: 106 - protein, 249 aa, Artificial Sequence
  Description of Artificial Sequence Synthetic polypeptide
EVQLVESGGGLVQPGGSLKLSCAASGFTFNKYAMNWVRQAPGKGLEWVARIRSKYNNYATYYADSVKSRFTISRDDSKNTAYLQMNNLKTEDTAVYYCVRHGNFGNSYTSYYAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 107 - protein, 249 aa, Artificial Sequence
  Description of Artificial Sequence Synthetic polypeptide
EVQLLESGGGLVQPGGSLKLSCAASGFTFNKYAMNWVRQAPGKGLEWVARIRSKYNNYATYYADSVKSRFTISRDDSKNTAYLQMNNLKTEDTAVYYCVRHGNFGNSYTSYYAYWGQGTLVTVSSGGGGSGGGGSGGGGSELVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 108 - protein, 14 aa, Artificial Sequence
  Description of Artificial Sequence Synthetic peptide
RSSTGAVTSGYYPN

SEQ ID NO: 109 - protein, 7 aa, Artificial Sequence
  Description of Artificial Sequence Synthetic peptide
ATDMRPS

SEQ ID NO: 110 - protein, 9 aa, Artificial Sequence
  Description of Artificial Sequence Synthetic peptide
ALWYSNRWV

SEQ ID NO: 111 - protein, 5 aa, Artificial Sequence
  Description of Artificial Sequence Synthetic peptide
GYAMN

SEQ ID NO: 112 - protein, 19 aa, Artificial Sequence
  Description of Artificial Sequence Synthetic peptide
RIRSKYNNYATYYADSVKE

SEQ ID NO: 113 - protein, 14 aa, Artificial Sequence
  Description of Artificial Sequence Synthetic peptide
HRNFGNSYLSWFAY

SEQ ID NO: 114 - protein, 125 aa, Artificial Sequence
  Description of Artificial Sequence Synthetic polypeptide
EVQLVESGGGLVQPGGSLKLSCAASGFTFNGYAMNWVRQAPGKGLEWVARIRSKYNNYATYYADSVKERFTISRDDSKNTAYLQMNNLKTEDTAVYYCVRHRNFGNSYLSWFAYWGQGTLVTVSS

SEQ ID NO: 115 - protein, 109 aa, Artificial Sequence; identical to SEQ ID NO: 94
  Description of Artificial Sequence Synthetic polypeptide

SEQ ID NO: 116 - protein, 125 aa, Artificial Sequence
  Description of Artificial Sequence Synthetic polypeptide
EVQLLESGGGLVQPGGSLKLSCAASGFTFNGYAMNWVRQAPGKGLEWVARIRSKYNNYATYYADSVKERFTISRDDSKNTAYLQMNNLKTEDTAVYYCVRHRNFGNSYLSWFAYWGQGTLVTVSS

SEQ ID NO: 117 - protein, 109 aa, Artificial Sequence; identical to SEQ ID NO: 96
  Description of Artificial Sequence Synthetic polypeptide

SEQ ID NO: 118 - protein, 249 aa, Artificial Sequence
  Description of Artificial Sequence Synthetic polypeptide
EVQLVESGGGLVQPGGSLKLSCAASGFTFNGYAMNWVRQAPGKGLEWVARIRSKYNNYATYYADSVKERFTISRDDSKNTAYLQMNNLKTEDTAVYYCVRHRNFGNSYLSWFAYWGQGTLVTVSSGGGGSGGGGSGGGGSQTVVTQEPSLTVSPGGTVTLTCRSSTGAVTSGYYPNWVQQKPGQAPRGLIGATDMRPSGTPARFSGSLLGGKAALTLSGVQPEDEAEYYCALWYSNRWVFGGGTKLTVL

SEQ ID NO: 119 - protein, 249 aa, Artificial Sequence
  Description of Artificial Sequence Synthetic polypeptide
EVQLLESGGGLVQPGGSLKLSCAASGFTFNGYAMNWVRQAPGKGLEWVARIRSKYNNYATYYADSVKERFTISRDDSKNTAYLQMNNLKTEDTAVYYCVRHRNFGNSYLSWFAYWGQGTLVTVSSGGGGSGGGGSGGGGSELVVTQEPSLTVSPGGTVTLTCRSSTGAVTSGYYPNWVQQKPGQAPRGLIGATDMRPSGTPARFSGSLLGGKAALTLSGVQPEDEAEYYCALWYSNRWVFGGGTKLTVL

SEQ ID NO: 120 - protein, 5 aa, Artificial Sequence
  Description of Artificial Sequence Synthetic peptide
VYAMN

SEQ ID NO: 121 - protein, 19 aa, Artificial Sequence
  Description of Artificial Sequence Synthetic peptide
RIRSKYNNYATYYADSVKK

SEQ ID NO: 122 - protein, 14 aa, Artificial Sequence
  Description of Artificial Sequence Synthetic peptide
HGNFGNSYISWWAY

SEQ ID NO: 123 - protein, 125 aa, Artificial Sequence
  Description of Artificial Sequence Synthetic polypeptide
EVQLVESGGGLVQPGGSLKLSCAASGFTFNVYAMNWVRQAPGKGLEWVARIRSKYNNYATYYADSVKKRFTISRDDSKNTAYLQMNNLKTEDTAVYYCVRHGNFGNSYISWWAYWGQGTLVTVSS

SEQ ID NO: 124 - protein, 109 aa, Artificial Sequence; identical to SEQ ID NO: 55
  Description of Artificial Sequence Synthetic polypeptide

SEQ ID NO: 125 - protein, 125 aa, Artificial Sequence
  Description of Artificial Sequence Synthetic polypeptide
EVQLLESGGGLVQPGGSLKLSCAASGFTFNVYAMNWVRQAPGKGLEWVARIRSKYNNYATYYADSVKKRFTISRDDSKNTAYLQMNNLKTEDTAVYYCVRHGNFGNSYISWWAYWGQGTLVTVSS

SEQ ID NO: 126 - protein, 109 aa, Artificial Sequence; identical to SEQ ID NO: 57
  Description of Artificial Sequence Synthetic polypeptide

SEQ ID NO: 127 - protein, 249 aa, Artificial Sequence
  Description of Artificial Sequence Synthetic polypeptide
EVQLVESGGGLVQPGGSLKLSCAASGFTFNVYAMNWVRQAPGKGLEWVARIRSKYNNYATYYADSVKKRFTISRDDSKNTAYLQMNNLKTEDTAVYYCVRHGNFGNSYISWWAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 128 - protein, 249 aa, Artificial Sequence
  Description of Artificial Sequence Synthetic polypeptide
EVQLLESGGGLVQPGGSLKLSCAASGFTFNVYAMNWVRQAPGKGLEWVARIRSKYNNYATYYADSVKKRFTISRDDSKNTAYLQMNNLKTEDTAVYYCVRHGNFGNSYISWWAYWGQGTLVTVSSGGGGSGGGGSGGGGSELVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 129 - protein, 14 aa, Artificial Sequence
  Description of Artificial Sequence Synthetic peptide
GSSTGAVTSGNYPN

SEQ ID NO: 130 - protein, 7 aa, Artificial Sequence
  Description of Artificial Sequence Synthetic peptide
GTKFLAP

SEQ ID NO: 131 - protein, 9 aa, Artificial Sequence
  Description of Artificial Sequence Synthetic peptide
VLWYSNRWV

SEQ ID NO: 132 - protein, 5 aa, Artificial Sequence
  Description of Artificial Sequence Synthetic peptide
SYAMN

SEQ ID NO: 133 - protein, 19 aa, Artificial Sequence
  Description of Artificial Sequence Synthetic peptide
RIRSKYNNYATYYADSVKG

SEQ ID NO: 134 - protein, 14 aa, Artificial Sequence
  Description of Artificial Sequence Synthetic peptide
HGNFGNSYVSWWAY

SEQ ID NO: 135 - protein, 125 aa, Artificial Sequence
  Description of Artificial Sequence Synthetic polypeptide
EVQLVESGGGLVQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYVSWWAYWGQGTLVTVSS

SEQ ID NO: 136 - protein, 109 aa, Artificial Sequence; identical to SEQ ID NO: 38
  Description of Artificial Sequence Synthetic polypeptide

SEQ ID NO: 137 - protein, 125 aa, Artificial Sequence
  Description of Artificial Sequence Synthetic polypeptide
EVQLLESGGGLVQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYVSWWAYWGQGTLVTVSS

SEQ ID NO: 138 - protein, 109 aa, Artificial Sequence
  Description of Artificial Sequence Synthetic polypeptide
ELVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 139 - protein, 249 aa, Artificial Sequence
  Description of Artificial Sequence Synthetic polypeptide
EVQLVESGGGLVQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYVSWWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 140 - protein, 249 aa, Artificial Sequence
  Description of Artificial Sequence Synthetic polypeptide
EVQLLESGGGLVQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYVSWWAYWGQGTLVTVSSGGGGSGGGGSGGGGSELVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 141 - protein, 5 aa, Artificial Sequence
  Description of Artificial Sequence Synthetic peptide
KYAMN

SEQ ID NO: 142 - protein, 19 aa, Artificial Sequence
  Description of Artificial Sequence Synthetic peptide
RIRSKYNNYATYYADSVKD

SEQ ID NO: 143 - protein, 14 aa, Artificial Sequence
  Description of Artificial Sequence Synthetic peptide
HGNFGNSYISYWAY

SEQ ID NO: 144 - protein, 125 aa, Artificial Sequence; identical to SEQ ID NO: 66
  Description of Artificial Sequence Synthetic polypeptide

SEQ ID NO: 145 - protein, 109 aa, Artificial Sequence; identical to SEQ ID NO: 38
  Description of Artificial Sequence Synthetic polypeptide

SEQ ID NO: 146 - protein, 125 aa, Artificial Sequence; identical to SEQ ID NO: 68
  Description of Artificial Sequence Synthetic polypeptide

SEQ ID NO: 147 - protein, 109 aa, Artificial Sequence; identical to SEQ ID NO: 138
  Description of Artificial Sequence Synthetic polypeptide

SEQ ID NO: 148 - protein, 249 aa, Artificial Sequence
  Description of Artificial Sequence Synthetic polypeptide
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 149 - protein, 249 aa, Artificial Sequence
  Description of Artificial Sequence Synthetic polypeptide
EVQLLESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSELVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 150 - protein, 453 aa, Artificial Sequence
  Description of Artificial Sequence Synthetic polypeptide
EVQLVQSGAEVKKPGESLKISCKGSGYSFTNYWIGWVRQMPGKGLEWMAIINPRDSDTRYRPSFQGQVTISADKSISTAYLQWSSLKASDTAMYYCARHGRGYEGYEGAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 151 - protein, 214 aa, Artificial Sequence
  Description of Artificial Sequence Synthetic polypeptide
DIQMTQSPSSLSASVGDRVTITCRSSQGIRSDLGWYQQKPGKAPKLLIYDASNLETGVPSRFSGSGSGTDFTLTISSLQPEDFATYYCQQANGFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 152 - protein, 451 aa, Artificial Sequence
  Description of Artificial Sequence Synthetic polypeptide
EVQLVESGGGLVQPGGSLRLSCAASGFTFSDADMDWVRQAPGKGLEWVGRTRNKAGSYTTEYAASVKGRFTISRDDSKNSLYLQMNSLKTEDTAVYYCAREPKYWIDFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 153 - protein, 214 aa, Artificial Sequence
  Description of Artificial Sequence Synthetic polypeptide
DIQMTQSPSSLSASVGDRVTITCRASQSISSYLNWYQQKPGKAPKLLIYAASSLQSGVPSRFSGSGSGTDFTLTISSLQPEDFATYYCQQSYIAPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC